SEQUENCE LISTING (110 sequences)

SEQ ID NO: 1 - protein, 173 aa, synthetic construct
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 2 - protein, 153 aa, synthetic construct
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 3 - protein, 108 aa, synthetic construct
DIQMTQSPSSLSASVGDRVTITCRASQSISSYLNWYQQKPGKAPKLLIYVASSLQSGVPSRFSGSGSGTNFTLTISSLQPEDFATYYCQQSYSTPITFGQGTRLEIKR

SEQ ID NO: 4 - protein, 117 aa, synthetic construct
QVQLQESGPGLVKPSETLSLTCTVSGDSISSYFWSWIRQPPGKGLEWIGYIYYSGTTNYNPSLKSRLTISVDTSKNQFSLKLNSVTAADTAVYYCARDWGNSPFDYWGQGTLVTVSS

SEQ ID NO: 5 - protein, 8 aa, synthetic construct
LSGRSDNH

SEQ ID NO: 6 - protein, 261 aa, synthetic construct
DIQMTQSPSSLSASVGDRVTITCRASQSISSYLNWYQQKPGKAPKLLIYVASSLQSGVPSRFSGSGSGTNFTLTISSLQPEDFATYYCQQSYSTPITFGQGTRLEIKRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 7 - protein, 290 aa, synthetic construct
QVQLQESGPGLVKPSETLSLTCTVSGDSISSYFWSWIRQPPGKGLEWIGYIYYSGTTNYNPSLKSRLTISVDTSKNQFSLKLNSVTAADTAVYYCARDWGNSPFDYWGQGTLVTVSSDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 8 - protein, 9 aa, synthetic construct
GDVEXNPGP

SEQ ID NO: 9 - DNA, 24 bp, synthetic construct
  source 1..24: mol_type = other DNA
gattacaaagacgatgacgataag

SEQ ID NO: 10 - DNA, 30 bp, synthetic construct
  source 1..30: mol_type = other DNA
gaacagaaactgatttccgaggaagatctg

SEQ ID NO: 11 - protein, 23 aa, synthetic construct
GSGSGEGRGSLLTCGDVEENPGP

SEQ ID NO: 12 - protein, 7 aa, synthetic construct
GQSSRSS

SEQ ID NO: 13 - protein, 6 aa, synthetic construct
GSGSGS

SEQ ID NO: 14 - protein, 10 aa, synthetic construct
GGGGSGGGGS

SEQ ID NO: 15 - protein, 20 aa, synthetic construct
GGGGSGGGGSGGGGSGGGGS

SEQ ID NO: 16 - protein, 15 aa, synthetic construct
GGGGSGGGGSGGGGS

SEQ ID NO: 17 - protein, 123 aa, synthetic construct
QVQLQQWGAGLLKPSETLSLTCAVYGGSFSGYYWSWIRQPPGKGLEWIGEIIHSGSTNYNPSLKSRVTISVDTSKNQFSLKLSSVTAADTAVYYCARRITMVRGVIVNAFDIWGQGTMVTVSS

SEQ ID NO: 18 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 108 aa, synthetic construct
DIQMTQSPSSLSASVGDRVSITCQASQDISHYLNWYQQKPGKAPKLLIYDASNLETGVPSRFSGGGSGTDFTFTISSLQPADIATYYCQQYDHLPYTFGQGTKLEIKR

SEQ ID NO: 20 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
GYYWS

SEQ ID NO: 22 - protein, 16 aa, synthetic construct
EIIHSGSTNYNPSLKS

SEQ ID NO: 23 - protein, 15 aa, synthetic construct
RITMVRGVIVNAFDI

SEQ ID NO: 24 - protein, 11 aa, synthetic construct
QASQDISHYLN

SEQ ID NO: 25 - protein, 7 aa, synthetic construct
DASNLET

SEQ ID NO: 26 - protein, 9 aa, synthetic construct
QQYDHLPYT

SEQ ID NO: 27 - protein, 11 aa, synthetic construct
RASQSISSYLN

SEQ ID NO: 28 - protein, 7 aa, synthetic construct
VASSLQS

SEQ ID NO: 29 - protein, 9 aa, synthetic construct
QQSYSTPIT

SEQ ID NO: 30 - protein, 5 aa, synthetic construct
SYFWS

SEQ ID NO: 31 - protein, 16 aa, synthetic construct
YIYYSGTTNYNPSLKS

SEQ ID NO: 32 - protein, 9 aa, synthetic construct
DWGNSPFDY

SEQ ID NO: 33 - DNA, 1926 bp, synthetic construct
  source 1..1926: mol_type = other DNA
atggccttaccagtgaccgccttgctcctgccgctggccttgctgctccacgccgccaggccggaacagaaactgatttccgaggaagatctggatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggaaataaagcgtagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataaggaggtgaagctgatggagtctgggggaggcttagtgaagcctggagggtccctgaaactctcctgtgcagcctctggattcactttcagtagctatgccatgtcttgggttcgccatactccggagaagaggctggagtgggtcgcaaccattagtagtggtggtggttacacctactatccagacagtgtgaagggtcgattcaccatctccagagacaatgccaacaacatcctgcacctgcaaatgagcagtctgaggtctgaggacacggccatgtattactgtacaagaggggagggactgggacgagctatggactactggggtcaaggaacctcagtcaccgtctcctcagacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctag

SEQ ID NO: 34 - DNA, 4851 bp, synthetic construct
  source 1..4851: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgcaagttcaactgcaacagtggggagcggggctgttgaagccctctgaaactctcagtctgacctgcgccgtgtacggaggctcattttccgggtattactggagttggatacggcagccgccgggcaaggggcttgagtggatcggggaaatcatacacagcggcagcacaaattataaccctagtcttaaaagcagggttacaatcagcgttgacacgagtaagaaccagttcagccttaagctgagcagtgtgactgcggcggacacggcggtgtattactgtgcgaggcgcattactatggtgcgaggagtaatcgtgaacgcgttcgatatatggggtcagggcacgatggtcactgtctcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 35 - DNA, 4175 bp, synthetic construct
  source 1..4175: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaacatgtacaggatgcaactcctgtcttgcattgcactaagtcttgcacttgtcacgaattcagatattcagatgactcaatcaccatcaagtctgagtgcatccgttggagacagggtaagcataacttgtcaagcgtcacaagatataagccattacctgaattggtaccagcagaaacccggaaaggcccctaagttgctcatatacgacgcaagcaaccttgaaacgggtgtacctagtagatttagtggaggtggctcaggcacggatttcacgtttactatttcatctctccagcctgctgatattgctacttattattgccaacagtacgaccatcttccgtacacatttggacagggtaccaaacttgagataaaacggacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagttcctggtgtacttgagggggatgagttcctcaatggtggttttgaccagcttgccattcatctcaatgagcacaaagcagtcaggagcatagtcagagatgagctctctgcacatgccacaggggctgaccaccctgatggatctgtccacctcatcagagtaggggtgcctgacagccacaatggtgtcaaagtccttctgcccgttgctcacagcagacccaatggcaatggcttcagcacagacagtgaccctgccaatgtaggcctcaatgtggacagcagagatgatctccccagtcttggtcctgatggccgccccgacatggtgcttgttgtcctcatagagcatggtgatcttctcagtggcgacctccaccagctccagatcctgctgagagatgttgaaggtcttcatgatggctcctcctgtcaggagaggaaagagaagaaggttagtacaattgctatagtgagttgtattatactatgcttatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 36 - DNA, 7022 bp, synthetic construct
  source 1..7022: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttctagaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaatttctactaagtgtagatgagtctctcagctggtacactaatttctactaagtgtagatggtgtgggagatctctgcttctgtaatttctactaagtgtagatatccatccgacattgaagttgactaatttctactaagtgtagatttttttaagcttggcgtggatccgatatcaactagatcttgagacaaggtacgatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagatttcaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccgtcacgagcagctggtttctaatatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgacccagcgcttaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagacgtacggccaccatggccttaccagtgaccgccttgctcctgccgctggccttgctgctccacgccgccaggccggaacagaaactgatttccgaggaagatctggatgttgtgatgacccaatccccgttgtccctgccggtaactcttgggcaaccggcttccatctcctgtcgcagttctcagtccttggtctattctgatggtaacacctatctcaactggttccaacagcgccccgggcaaagcccaaggaggctcatctatcgggttagtaatagggattctggtgtccctgaccgcttcagtgggtcaggttccgggaccgacttcacccttaaaatttcacgagttgaggcggaggacgtaggagtctactactgcatgcaaggcacacattggcctctgacgttcggtggaggtacgaaggtcgagattaagcgaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataagcaagttcagcttgtggaaagcgggggaggcgtcgttcaacccggtcgatcattgcgcctctcttgtgctgcatcaggcttcacgttctctagttacggcatgcactgggttagacaagcaccaggtaagggcctcgaatgggtcgctgtaatctggtatgatgtgtctaaccaatactatgcagacagcgtaaagggccggttcacgataagcggggataacagtaagaacacactctatttgcaactcaattctttgcgggccgaagatacggccgtgtattattgcgcgagggactacttcgatttgtgggggcggggcactctggttaccgtatccagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggctagcaataaaagatctttattttcattagatctgtgtgttggttttttgtgtggtaccgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagcagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacggaaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccacaattcgatatcaagcttaataaaagatctttattttcattagatctgtgtgttggttttttgtgtggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 37 - DNA, 7016 bp, synthetic construct
  source 1..7016: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttctagaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaatttctactaagtgtagatgagtctctcagctggtacactaatttctactaagtgtagatggtgtgggagatctctgcttctgtaatttctactaagtgtagatatccatccgacattgaagttgactaatttctactaagtgtagatttttttaagcttggcgtggatccgatatcaactagatcttgagacaaggtacgatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagatttcaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccgtcacgagcagctggtttctaatatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgacccagcgcttaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagacgtacggccaccatggccttaccagtgaccgccttgctcctgccgctggccttgctgctccacgccgccaggccggaacagaaactgatttccgaggaagatctggacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataagcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggctagcaataaaagatctttattttcattagatctgtgtgttggttttttgtgtggtaccgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagcagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacggaaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccacaattcgatatcaagcttaataaaagatctttattttcattagatctgtgtgttggttttttgtgtggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 38 - DNA, 7022 bp, synthetic construct
  source 1..7022: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttctagaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaatttctactaagtgtagatgagtctctcagctggtacactaatttctactaagtgtagatggtgtgggagatctctgcttctgtaatttctactaagtgtagatatccatccgacattgaagttgactaatttctactaagtgtagatttttttaagcttggcgtggatccgatatcaactagatcttgagacaaggtacgatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagatttcaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccgtcacgagcagctggtttctaatatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgacccagcgcttaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagacgtacggccaccatggccttaccagtgaccgccttgctcctgccgctggccttgctgctccacgccgccaggccggaacagaaactgatttccgaggaagatctggatgttgtgatgacccagtcaccgctctctctcccagtcacacttggtcaacctgcatcaatctcatgccgctcctcacaatctctcgtatactctgatggcaacacgtatttgaattggtttcaacaacggcccggacaaagtcctagacgactcatttacaaggtgtctaatcgagactctggcgttcctgaccgattttccggtagtggcagtgggaccgatttcacgttgaagatatcacgagtggaggccgaagatgtcggtgtctactactgtatgcaaggaacccattggcctttgacttttggaggcggcacgaaggtcgaaatcaaaagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataagcaagtccagcttgtggaaagtggagggggggttgttcagccaggaaggagtctcagactgtcatgcgcggcgtcaggatttactttctctagctacgggatgcactgggtaagacaggcgcctggaaagggcctcgagtgggtcgcggtcatatggtacgacgtaaataatcagtattatgtagacagcgtgaaggggagatttacaatatctggggacatttcaaagaacactatgtaccttcaaatgaatagtttgcggaccgaagatacggctctgtatttttgcgccagagattatttcgattattggggtcagggaacccttgtcactgtaagttcagacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggctagcaataaaagatctttattttcattagatctgtgtgttggttttttgtgtggtaccgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagcagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacggaaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccacaattcgatatcaagcttaataaaagatctttattttcattagatctgtgtgttggttttttgtgtggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 39 - DNA, 6853 bp, synthetic construct
  source 1..6853: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcattggatcagtggatccgatatcaactagatcttgagacaaggtacgatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagatttcaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccgtcacgagcagctggtttctaatatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgacccagcgcttaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagacgtacggccaccatggccttaccagtgaccgccttgctcctgccgctggccttgctgctccacgccgccaggccggaacagaaactgatttccgaggaagatctggacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgcagcaaacgcagccgcctgctgcatagcgattatatgaacatgaccccgcgccgcccgggcccgacccgcaaacattatcagccgtatgcgccgccacgcgattttgcggcgtatcgcagccgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataagcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagcgccctgtacctgctccggagggaccagaggctgccccccgatgcccacaagccccctgggggaggcagtttccggacccccatccaagaggagcaggccgacgcccactccaccctggccaagatctaggctagcaataaaagatctttattttcattagatctgtgtgttggttttttgtgtggtaccgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagcagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacggaaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccacaattcgatatcaagcttaataaaagatctttattttcattagatctgtgtgttggttttttgtgtggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 40 - DNA, 7009 bp, synthetic construct
  source 1..7009: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcattggatcagtggatccgatatcaactagatcttgagacaaggtacgatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagatttcaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccgtcacgagcagctggtttctaatatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgacccagcgcttaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagacgtacggccaccatggccttaccagtgaccgccttgctcctgccgctggccttgctgctccacgccgccaggccggaacagaaactgatttccgaggaagatctggacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgcagcaaacgcagccgcctgctgcatagcgattatatgaacatgaccccgcgccgcccgggcccgacccgcaaacattatcagccgtatgcgccgccacgcgattttgcggcgtatcgcagccgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataagcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagcaactgccgcaacacaggtccgtggcttaaaaaggtcttgaaatgcaatacgcctgacccgtcaaagttcttctcacagctttcctctgaacatggcggggacgtacaaaagtggttgtctagtccctttccttcaagttctttcagcccagggggcctggcaccagaaatctctccactggaggttcttgagagagacaaagtcactcagttgctgccgcttaacactgatgcgtatctttcactccaggaacttcaaggccaagatcctacgcacctggtctaggctagcaataaaagatctttattttcattagatctgtgtgttggttttttgtgtggtaccgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagcagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacggaaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccacaattcgatatcaagcttaataaaagatctttattttcattagatctgtgtgttggttttttgtgtggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 41 - DNA, 6841 bp, synthetic construct
  source 1..6841: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcattggatcagtggatccgatatcaactagatcttgagacaaggtacgatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagatttcaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccgtcacgagcagctggtttctaatatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgacccagcgcttaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagacgtacggccaccatggccttaccagtgaccgccttgctcctgccgctggccttgctgctccacgccgccaggccggaacagaaactgatttccgaggaagatctggacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgcagcaaacgcagccgcctgctgcatagcgattatatgaacatgaccccgcgccgcccgggcccgacccgcaaacattatcagccgtatgcgccgccacgcgattttgcggcgtatcgcagccgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataagcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctgttggcttacaaaaaagaagtattcatccagtgtgcacgaccctaacggtgaatacatgttcatgagagcagtgaacacagccaaaaaatctagactcacagatgtgaccctataggctagcaataaaagatctttattttcattagatctgtgtgttggttttttgtgtggtaccgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagcagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacggaaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccacaattcgatatcaagcttaataaaagatctttattttcattagatctgtgtgttggttttttgtgtggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 42 - DNA, 7312 bp, synthetic construct
  source 1..7312: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcattggatcagtggatccgatatcaactagatcttgagacaaggtacgatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagatttcaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccgtcacgagcagctggtttctaatatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgacccagcgcttaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagacgtacggccaccatggccttaccagtgaccgccttgctcctgccgctggccttgctgctccacgccgccaggccggaacagaaactgatttccgaggaagatctggacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgcagcaaacgcagccgcctgctgcatagcgattatatgaacatgaccccgcgccgcccgggcccgacccgcaaacattatcagccgtatgcgccgccacgcgattttgcggcgtatcgcagccgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataagcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagcaagaaaaggatcaaaccaatcgtctggccgtcattgcctgatcataagaagacactggaacacctgtgcaagaaaccgagaaagaatcttaatgtaagcttcaaccccgaaagcttcctggactgtcaaatccatagggtggacgatatacaggcaagagacgaagtcgaaggtttccttcaagacacgttcccgcaacaactggaggagtctgaaaagcaaaggttgggcggcgacgttcaatcaccaaattgtcccagtgaagatgtagttattacgcccgaatcctttgggagggactcaagtctgacctgtctggctggcaatgtgagcgcatgtgatgccccgatactgtcttcaagtcgctctttggattgtcgagagtccggcaaaaacggtcctcatgtgtatcaggacttgcttttgtccttggggacgaccaacagcacgttgccacctcctttcagcctccagtccggtatcctgacgttgaacccagtagctcaaggccagcctatccttacgagcctcgggagtaatcaagaagaggcttatgtgaccatgagcagtttttaccagaaccagtaggctagcaataaaagatctttattttcattagatctgtgtgttggttttttgtgtggtaccgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagcagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacggaaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccacaattcgatatcaagcttaataaaagatctttattttcattagatctgtgtgttggttttttgtgtggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 43 - DNA, 4212 bp, synthetic construct
  source 1..4212: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgcaccatcaccatcaccatcaccatgagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 44 - DNA, 8168 bp, synthetic construct
  source 1..8168: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagatcacgacattactatatatataatataggaagcatttaatagaacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgccgatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaatcaacactctcccctgttgaagctctttgtgacgggcgaggacaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccaccgtacgtcgcttaatctcgaccttcgtacctccaccgaacgtcagaggccaatgtgtgccttgcatgcagtagtagactcctacgtcctccgcctcaactcgtgaaattttaagggtgaagtcggtcccggaacctgacccactgaagcggtcagggacaccagaatccctattactaacccgatagatgagcctccttgggctttgcccggggcgctgttggaaccagttgagataggtgttaccatcagaatagaccaaggactgagaactgcgacaggagatggaagccggttgcccaagagttaccggcagggacaacggggattgggtcatcacaacatcgagctctgccaagacgcttgctatgacggaaaaaatactaaagcaccttaacagctgcatgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccatgcagttacttcgctgtttttcaatattttctgttattgccagcgttttagcactcgagcaagttcagcttgtggaaagcgggggaggcgtcgttcaacccggtcgatcattgcgcctctcttgtgctgcatcaggcttcacgttctctagttacggcatgcactgggttagacaagcaccaggtaagggcctcgaatgggtcgctgtaatctggtatgatgtgtctaaccaatactatgcagacagcgtaaagggccggttcacgataagcggggataacagtaagaacacactctatttgcaactcaattctttgcgggccgaagatacggccgtgtattattgcgcgagggactacttcgatttgtgggggcggggcactctggttaccgtatccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagtctgcaagctagtggtggtggtggttctggtggtggtggttctggtggtggtggttcttacccatacgacgttccagactacgctggcggtggaggcagtcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttggcagtgaacaaaaacttatttctgaagaagatctgtagcctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 45 - DNA, 8162 bp, synthetic construct
  source 1..8162: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagatcacgacattactatatatataatataggaagcatttaatagaacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgccgatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaatcaacactctcccctgttgaagctctttgtgacgggcgaggacaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccaccgtacggcgctttatctccagtctcgtcccttgcccaaaggtaattggggttgaataagactgctgacaatagtaagttgcaaagtcctctggctggagtgaggaaatagtgagtgtgaagttggtaccggaaccggacccagaaaaccgacttggtacgccactctgaagagaagaggcaacataaatcaaaagtttcggagcttttccgggcttctgttgataccaattaaggtatgatgaaatagattgactagctcggcaggtgatcgttacccggtcacccacggaagcggacagactggacggggactgcgtcatttgaatgtcgagctctgccaagacgcttgctatgacggaaaaaatactaaagcaccttaacagctgcatgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccatgcagttacttcgctgtttttcaatattttctgttattgccagcgttttagcactcgagcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagtctgcaagctagtggtggtggtggttctggtggtggtggttctggtggtggtggttcttacccatacgacgttccagactacgctggcggtggaggcagtcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttggcagtgaacaaaaacttatttctgaagaagatctgtagcctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 46 - DNA, 8168 bp, synthetic construct
  source 1..8168: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagatcacgacattactatatatataatataggaagcatttaatagaacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgccgatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaatcaacactctcccctgttgaagctctttgtgacgggcgaggacaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccaccgtacgtcttttgatttcgaccttcgtgccgcctccaaaagtcaaaggccaatgggttccttgcatacagtagtagacaccgacatcttcggcctccactcgtgatatcttcaacgtgaaatcggtcccactgccactaccggaaaatcggtcaggaacgccagagtctcgattagacaccttgtaaatgagtcgtctaggactttgtccgggccgttgttgaaaccaattcaaatacgtgttgccatcagagtatacgagagattgtgaggagcggcatgagattgatgcaggttgaccaagtgtgactgggagagagagcggtgactgggtcatcacaacatcgagctctgccaagacgcttgctatgacggaaaaaatactaaagcaccttaacagctgcatgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccatgcagttacttcgctgtttttcaatattttctgttattgccagcgttttagcactcgagcaagtccagcttgtggaaagtggagggggggttgttcagccaggaaggagtctcagactgtcatgcgcggcgtcaggatttactttctctagctacgggatgcactgggtaagacaggcgcctggaaagggcctcgagtgggtcgcggtcatatggtacgacgtaaataatcagtattatgtagacagcgtgaaggggagatttacaatatctggggacatttcaaagaacactatgtaccttcaaatgaatagtttgcggaccgaagatacggctctgtatttttgcgccagagattatttcgattattggggtcagggaacccttgtcactgtaagttcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagtctgcaagctagtggtggtggtggttctggtggtggtggttctggtggtggtggttcttacccatacgacgttccagactacgctggcggtggaggcagtcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttggcagtgaacaaaaacttatttctgaagaagatctgtagcctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 47 - DNA, 11142 bp, synthetic construct
  source 1..11142: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaacagaaactgatttccgaggaagatctgtcatacgtgctcactcagccgccctcagttagtgtggctccaggccagacggcccgcataacttgtgggggcaacaacatcggctccaagtctgttcattggtatcaacaacctccaggtcaggccccagttgtcgttgtgtacgacgattctgatcggccatcaggcataccggaacggttctcaggttcaaactccggcaacacggctactctcactatatcacgcgtagaggcaggggacgaagcggtttattattgccaggtctgggactcatctagcgatcacgtcgtattcggcggggggacaaagctgacggtcctaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataagcagcttcagctccaagaaagtggacctggccttgtcaaaccctcagaaactctgtcacttacatgtactgtgtcaggcggaagcatcagctcaggaagctatttttggggatggatcaggcagcctccagggaaggggctggaatggattggttctatctattacagtggaatcacttactacaatccttctttgaaatcccgagtgacgattagtgtggatactagtaagaatcagttcagtttgaagttgagcagcgtgaccgccgccgataccgccgtttattactgtgccagacacgacggagcagtggcgggcttgtttgactattggggacaggggacattggttacggtctcttctgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 48 - DNA, 11136 bp, synthetic construct
  source 1..11136: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaacagaaactgatttccgaggaagatctggatgttgtgatgacccaatccccgttgtccctgccggtaactcttgggcaaccggcttccatctcctgtcgcagttctcagtccttggtctattctgatggtaacacctatctcaactggttccaacagcgccccgggcaaagcccaaggaggctcatctatcgggttagtaatagggattctggtgtccctgaccgcttcagtgggtcaggttccgggaccgacttcacccttaaaatttcacgagttgaggcggaggacgtaggagtctactactgcatgcaaggcacacattggcctctgacgttcggtggaggtacgaaggtcgagattaagcgaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataagcaagttcagcttgtggaaagcgggggaggcgtcgttcaacccggtcgatcattgcgcctctcttgtgctgcatcaggcttcacgttctctagttacggcatgcactgggttagacaagcaccaggtaagggcctcgaatgggtcgctgtaatctggtatgatgtgtctaaccaatactatgcagacagcgtaaagggccggttcacgataagcggggataacagtaagaacacactctatttgcaactcaattctttgcgggccgaagatacggccgtgtattattgcgcgagggactacttcgatttgtgggggcggggcactctggttaccgtatccagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 49 - DNA, 11129 bp, synthetic construct
  source 1..11129: mol_type = other DNA
tcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaacagaaactgatttccgaggaagatctggacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataagcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 50 - DNA, 11136 bp, synthetic construct
  source 1..11136: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaacagaaactgatttccgaggaagatctggatgttgtgatgacccagtcaccgctctctctcccagtcacacttggtcaacctgcatcaatctcatgccgctcctcacaatctctcgtatactctgatggcaacacgtatttgaattggtttcaacaacggcccggacaaagtcctagacgactcatttacaaggtgtctaatcgagactctggcgttcctgaccgattttccggtagtggcagtgggaccgatttcacgttgaagatatcacgagtggaggccgaagatgtcggtgtctactactgtatgcaaggaacccattggcctttgacttttggaggcggcacgaaggtcgaaatcaaaagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataagcaagtccagcttgtggaaagtggagggggggttgttcagccaggaaggagtctcagactgtcatgcgcggcgtcaggatttactttctctagctacgggatgcactgggtaagacaggcgcctggaaagggcctcgagtgggtcgcggtcatatggtacgacgtaaataatcagtattatgtagacagcgtgaaggggagatttacaatatctggggacatttcaaagaacactatgtaccttcaaatgaatagtttgcggaccgaagatacggctctgtatttttgcgccagagattatttcgattattggggtcagggaacccttgtcactgtaagttcagacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 51 - DNA, 11136 bp, synthetic construct
  source 1..11136: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaacagaaactgatttccgaggaagatctggatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggaaataaagcgtagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataaggaggtgaagctgatggagtctgggggaggcttagtgaagcctggagggtccctgaaactctcctgtgcagcctctggattcactttcagtagctatgccatgtcttgggttcgccatactccggagaagaggctggagtgggtcgcaaccattagtagtggtggtggttacacctactatccagacagtgtgaagggtcgattcaccatctccagagacaatgccaacaacatcctgcacctgcaaatgagcagtctgaggtctgaggacacggccatgtattactgtacaagaggggagggactgggacgagctatggactactggggtcaaggaacctcagtcaccgtctcctcagacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 52 - DNA, 11937 bp, synthetic construct
  source 1..11937: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaacagaaactgatttccgaggaagatctggatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggaaataaagcgtggtggtggtggttctggtggtggtggttctggcggcggcggctccggtggtggtggatccgaggtgaagctgatggagtctgggggaggcttagtgaagcctggagggtccctgaaactctcctgtgcagcctctggattcactttcagtagctatgccatgtcttgggttcgccatactccggagaagaggctggagtgggtcgcaaccattagtagtggtggtggttacacctactatccagacagtgtgaagggtcgattcaccatctccagagacaatgccaacaacatcctgcacctgcaaatgagcagtctgaggtctgaggacacggccatgtattactgtacaagaggggagggactgggacgagctatggactactggggtcaaggaacctcagtcaccgtctcctcaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataaggatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggaaataaagcgtggtggtggtggttctggtggtggtggttctggcggcggcggctccggtggtggtggatccgaggtgaagctgatggagtctgggggaggcttagtgaagcctggagggtccctgaaactctcctgtgcagcctctggattcactttcagtagctatgccatgtcttgggttcgccatactccggagaagaggctggagtgggtcgcaaccattagtagtggtggtggttacacctactatccagacagtgtgaagggtcgattcaccatctccagagacaatgccaacaacatcctgcacctgcaaatgagcagtctgaggtctgaggacacggccatgtattactgtacaagaggggagggactgggacgagctatggactactggggtcaaggaacctcagtcaccgtctcctcagacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 53 - DNA, 11937 bp, synthetic construct
  source 1..11937: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaacagaaactgatttccgaggaagatctggaggtgaagctgatggagtctgggggaggcttagtgaagcctggagggtccctgaaactctcctgtgcagcctctggattcactttcagtagctatgccatgtcttgggttcgccatactccggagaagaggctggagtgggtcgcaaccattagtagtggtggtggttacacctactatccagacagtgtgaagggtcgattcaccatctccagagacaatgccaacaacatcctgcacctgcaaatgagcagtctgaggtctgaggacacggccatgtattactgtacaagaggggagggactgggacgagctatggactactggggtcaaggaacctcagtcaccgtctcctcaggtggtggtggttctggtggtggtggttctggcggcggcggctccggtggtggtggatccgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggaaataaagcgtagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataaggatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggaaataaagcgtggtggtggtggttctggtggtggtggttctggcggcggcggctccggtggtggtggatccgaggtgaagctgatggagtctgggggaggcttagtgaagcctggagggtccctgaaactctcctgtgcagcctctggattcactttcagtagctatgccatgtcttgggttcgccatactccggagaagaggctggagtgggtcgcaaccattagtagtggtggtggttacacctactatccagacagtgtgaagggtcgattcaccatctccagagacaatgccaacaacatcctgcacctgcaaatgagcagtctgaggtctgaggacacggccatgtattactgtacaagaggggagggactgggacgagctatggactactggggtcaaggaacctcagtcaccgtctcctcagacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgttgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 54 - DNA, 11931 bp, synthetic construct
  source 1..11931: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaacagaaactgatttccgaggaagatctggacattgtgatgactcagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatccgtacacgttcggaggggggaccaagctggaaataaagcgtggtggtggtggttctggtggtggtggttctggcggcggcggctccggtggtggtggatcccaggtccaactgcagcagcctggggctgacctggtgaagcctagggcttcagtgaagctgtcctgcaaggcttctggctacaccttcaccagctactggatacactgggtgaggcagaggcctggccatggccttgagtggattggagagattaatcctagcaacggtcgaactaacaataatgagaagttcgagaacagggccaaactgactgtagacaaatcctccagcacagcctatatgcaactcagcagcctgacatctgaggactctgcggtctattactgtgcgagaggggaatactacggtagtgactactggggccaaggcaccactctcacagtctcctcaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgattacaaagacgatgacgataaggatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggaaataaagcgtggtggtggtggttctggtggtggtggttctggcggcggcggctccggtggtggtggatccgaggtgaagctgatggagtctgggggaggcttagtgaagcctggagggtccctgaaactctcctgtgcagcctctggattcactttcagtagctatgccatgtcttgggttcgccatactccggagaagaggctggagtgggtcgcaaccattagtagtggtggtggttacacctactatccagacagtgtgaagggtcgattcaccatctccagagacaatgccaacaacatcctgcacctgcaaatgagcagtctgaggtctgaggacacggccatgtattactgtacaagaggggagggactgggacgagctatggactactggggtcaaggaacctcagtcaccgtctcctcagacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 55 - DNA, 11364 bp, synthetic construct
  source 1..11364: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttgattccagtctccctcagatctggggacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgattccagtctccctcagatctgggcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 56 - DNA, 11301 bp, synthetic construct
  source 1..11301: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggctcaactggagaaagagcttcaggcactggagaaaaaactggcacaacttgaatggaaccaggccctggaaaaagaattggctcaaattccagtctccctcagatctggggacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgcgcaattgaagaaaaagctgcaggctaataaaaaagagcttgctcagcttaagtggaagcttcaggcgcttaagaagaagctggctcagattccagtctccctcagatctgggcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 57 - DNA, 11316 bp, synthetic construct
  source 1..11316: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggttgcccagctggaggagaaggtaaagacactgcgggccgagaattacgaactcaagtcagaggtgcaacgacttgaggagcaggtggcccaacttattccagtctccctcagatctggggacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgtagacgaacttcaggccgaggtcgatcaacttgaagatgagaattacgccttgaaaacgaaggttgctcaactcagaaaaaaggttgaaaagcttattccagtctccctcagatctgggcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 58 - DNA, 11364 bp, synthetic construct
  source 1..11364: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttgcttagcgggcggagcgacaaccacgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgcttagcgggcggagcgacaaccaccaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 59 - DNA, 11316 bp, synthetic construct
  source 1..11316: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggttgcccagctggaggagaaggtaaagacactgcgggccgagaattacgaactcaagtcagaggtgcaacgacttgaggagcaggtggcccaacttcttagcgggcggagcgacaaccacgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgtagacgaacttcaggccgaggtcgatcaacttgaagatgagaattacgccttgaaaacgaaggttgctcaactcagaaaaaaggttgaaaagcttcttagcgggcggagcgacaaccaccaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 60 - DNA, 11394 bp, synthetic construct
  source 1..11394: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttgcttagcgggcggagcgacaaccacggttccagcggaaccgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgcttagcgggcggagcgacaaccacggatcctcaggtacgcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 61 - DNA, 11346 bp, synthetic construct
  source 1..11346: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggttgcccagctggaggagaaggtaaagacactgcgggccgagaattacgaactcaagtcagaggtgcaacgacttgaggagcaggtggcccaacttcttagcgggcggagcgacaaccacggttccagcggaaccgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgtagacgaacttcaggccgaggtcgatcaacttgaagatgagaattacgccttgaaaacgaaggttgctcaactcagaaaaaaggttgaaaagcttcttagcgggcggagcgacaaccacggatcctcaggtacgcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 62 - DNA, 11454 bp, synthetic construct
  source 1..11454: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttggggggaggatcttctggcctccttagcggtagatcagacaatcacggtggtggctccggcggagggtctgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgggcggtgggagcagtggactgcttagtgggcggtcagacaatcacggtggcggcagcggaggaggctcacaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 63 - DNA, 11544 bp, synthetic construct
  source 1..11544: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttgggaggcggttcaggtggtggctcttcagggcttctgagcggacgatctgataaccattcagggctgttgagtggtcgctcagacaaccacggaggtggtagtggaggcggttctgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgggtggcggatcaggtggtggaagctctgggttgttgtccggaaggtcagataaccattctgggctcctctcaggacgatccgacaatcatggcggaggttcaggaggaggcagccaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 64 - DNA, 11610 bp, synthetic construct
  source 1..11610: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttgggaggtggttctggaggcggttcatccggtcttctcagtggacggagcgataatcattccggactgttgtcagggcgatccgataaccacagcggattgttgtctggtcgcagtgataatcatggtggagggagcggaggcggttctgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgggaggtggtagtgggggtggatcatcaggtctgctcagcggcaggagtgataaccactcagggctcctctctggcagatccgataatcatagcggtctgttgtctggtcgatctgataatcatggtggcgggtcaggaggcggcagtcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 65 - DNA, 11604 bp, synthetic construct
  source 1..11604: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttgcatcctgttggtttgttggcgagagtgcctctctcactgtactctggtcatccagttggtctccttgctagggtgcccttgagtctttattcagggctgtccggcagatctgataatcatggagggggttccggtggaggctcagacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgcaccccgttggtctgcttgcaagggttcctcttagcctgtatagtggacatcccgtagggcttcttgcacgagtgcctcttagtctttatagcggcctttcaggacgcagtgacaatcacgggggaggtagcggaggtgggtctcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 66 - DNA, 11412 bp, synthetic construct
  source 1..11412: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttgcttagcgggcggagcgacaaccaccttagtgggcggtcagacaatcacgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgcttagcgggcggagcgacaaccaccttagtgggcggtcagacaatcaccaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 67 - DNA, 11364 bp, synthetic construct
  source 1..11364: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggttgcccagctggaggagaaggtaaagacactgcgggccgagaattacgaactcaagtcagaggtgcaacgacttgaggagcaggtggcccaacttcttagcgggcggagcgacaaccaccttagtgggcggtcagacaatcacgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgtagacgaacttcaggccgaggtcgatcaacttgaagatgagaattacgccttgaaaacgaaggttgctcaactcagaaaaaaggttgaaaagcttcttagcgggcggagcgacaaccaccttagtgggcggtcagacaatcaccaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 68 - DNA, 11412 bp, synthetic construct
  source 1..11412: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttgcttagcgggcggagcgacaaccaccttagtgggcggtcagacaatcacgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgcatccagttggtctccttgctagggtgcccttgagtctttattcagggcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 69 - DNA, 11364 bp, synthetic construct
  source 1..11364: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggttgcccagctggaggagaaggtaaagacactgcgggccgagaattacgaactcaagtcagaggtgcaacgacttgaggagcaggtggcccaacttcttagcgggcggagcgacaaccaccttagtgggcggtcagacaatcacgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgtagacgaacttcaggccgaggtcgatcaacttgaagatgagaattacgccttgaaaacgaaggttgctcaactcagaaaaaaggttgaaaagcttcatccagttggtctccttgctagggtgcccttgagtctttattcagggcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 70 - DNA, 11370 bp, synthetic construct
  source 1..11370: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttgcttagcgggcggagcgacaaccacggtgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgcttagcgggcggagcgacaaccacggacaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 71 - DNA, 11376 bp, synthetic construct
  source 1..11376: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttgcttagcgggcggagcgacaaccacggtggagacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgcttagcgggcggagcgacaaccacggtggacaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 72 - DNA, 11382 bp, synthetic construct
  source 1..11382: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttgcttagcgggcggagcgacaaccacggtggatccgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgcttagcgggcggagcgacaaccacggtggatcccaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 73 - DNA, 11388 bp, synthetic construct
  source 1..11388: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggaggcttgtggtgcttctacgacggtagcccagctggaagaaaaagtaaagacacttagggccgaaaactacgaactgaagtctgaggtccagcgcttggaagagcaggtcgcgcagttgcttagcgggcggagcgacaaccacggtggagggtccgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgaggcttgcggtgcaagcacgtcagttgacgaattgcaagccgaggtggatcaactggaggacgaaaactatgcactcaagactaaagtggctcaactcaggaagaaagttgaaaagttgcttagcgggcggagcgacaaccacggtggagggtcccaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 74 - DNA, 11334 bp, synthetic construct
  source 1..11334: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccggctcaactggagaaagagcttcaggcactggagaaaaaactggcacaacttgaatgggaaaaccaggccctggaaaaagaattggctcaacttagcgggcggagcgacaaccacggttccagcggaaccgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccgcgcaattgaagaaaaagctgcaggctaataaaaaagagcttgctcagcttaagtggaagcttcaggcgcttaagaagaagctggctcagcttagcgggcggagcgacaaccacggatcctcaggtacgcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 75 - DNA, 11358 bp, synthetic construct
  source 1..11358: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggccttaccagtgaccgccttgctcctgccgctggccttgctgctccacgccgccaggccgggggagcttgaggagctcttgaaacatttgaaagaactgttgaaaggtccgaggaagggcgaactggaggaactgcttaagcatttgaaagagttgctgaagcttagcgggcggagcgacaaccacggttccagcggaaccgacattcaaatgacgcagtccccgtccagtctgtccgcttccgtgggtgaccgggtaacgatcacctgccgagctagtcaatctatttcatcataccttaattggtatcaacagaagcccggaaaagctccgaaacttttgatttatgttgcctcttctcttcagagtggcgtaccaagtcggttttctgggtccggttccggtaccaacttcacactcactatttcctcactccagccagaggactttgcaacttactattgtcagcagtcttattcaaccccaattacctttgggcaagggacgagactggagataaagcgcagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgcgacggaagagatcgggttccggcgccccggtgaaacagactttgaattttgaccttctcaagttggcgggagacgtggagtccaacccagggccgatgtggcttcagtcactccttctcttgggtactgttgcttgcagcatctccggggagttggaggaattgcttaaacatctgaaggagctgttgaagggtccgcgaaagggcgaactggaagagctgcttaaacatctcaaagagctgttgaagcttagcgggcggagcgacaaccacggatcctcaggtacgcaggtgcaactccaggaatctggccccggtcttgtgaaacccagcgaaacgctgtctctcacgtgtactgtcagtggtgattctatcagtagctacttctggagctggatacggcagccacccggcaaaggtcttgaatggattggatacatttactatagcggtactacaaactacaatccgagcttgaagtcacggctgacaatctctgtagacactagtaagaatcaattttctcttaaactgaatagcgtcaccgcagcggatacagccgtttactattgtgccagggattggggaaattctccttttgactattggggtcaaggtaccttggtaactgtatcaagcgacaagcagctggacgccgacgtgagccccaagcctaccatcttcctgcccagcatcgccgagaccaagctgcagaaggccggcacctacctgtgcctgctggaaaagttcttccccgacgtgatcaagatccactggcaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggatcccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 76 - DNA, 4212 bp, synthetic construct
  source 1..4212: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgcaccatcaccatcaccatcaccatgagcccaaatcttcagacaaaactcacacatgcccaccgtgcccagcacctgaactcctggggggaccgtcagtcttcctcttccccccaaaacccaaggacaccgcaatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggggccccccgagcaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacgcacactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 77 - DNA, 3244 bp, synthetic construct
  source 1..3244: mol_type = other DNA
aagtgcaaggtctccaacaaagccctcccagcccccatcgagaaaaccatctccaaagccaaagggcagccccgagaaccacaggtgtacaccctgcccccatccgcagaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtgggcccaggggaacgtcttctcatgctccgtgatgcatgaggctctgcacaaccactacacgcagaagagcctctccctgtctccggccgcatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 78-80 - skipped (3 entries)

SEQ ID NO: 81 - protein, 6 aa, synthetic construct
ASGGGS

SEQ ID NO: 82 - protein, 8 aa, synthetic construct
DYKDDDDK

SEQ ID NO: 83 - protein, 48 aa, synthetic construct
QRRKYRSNKGESPVEPAEPCHYSCPREEEGSTIPIQEDYRKPEPACSP

SEQ ID NO: 84 - protein, 41 aa, synthetic construct
RSKRSRLLHSDYMNMTPRRPGPTRKHYQPYAPPRDFAAYRS

SEQ ID NO: 85 - protein, 42 aa, synthetic construct
KRGRKKLLYIFKQPFMRPVQTTQEEDGCSCRFPEEEEGGCEL

SEQ ID NO: 86 - protein, 42 aa, synthetic construct
ALYLLRRDQRLPPDAHKPPGGGSFRTPIQEEQADAHSTLAKI

SEQ ID NO: 87 - protein, 189 aa, synthetic construct
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 88 - protein, 62 aa, synthetic construct
KKVAKKPTNKAPHPKQEPQEINFPDDLPGSNTAAPVQETLHGCQPVTQEDGKESRISVQERQ

SEQ ID NO: 89 - protein, 46 aa, synthetic construct
KALIHLSDLREYRRFEKEKLKSQWNNDNPLFKSATTTVMNPKFAES

SEQ ID NO: 90 - protein, 38 aa, synthetic construct
CWLTKKKYSSSVHDPNGEYMFMRAVNTAKKSRLTDVTL

SEQ ID NO: 91 - protein, 116 aa, synthetic construct
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 92 - protein, 39 aa, synthetic construct
RTQIKKLCSWRDKNSAACVVYEDMSHSRCNTLSSPNQYQ

SEQ ID NO: 93 - protein, 37 aa, synthetic construct
MEESVVRPSVFVVDGQTDIPFTRLGRSHRRQSCSVAR

SEQ ID NO: 94 - protein, 70 aa, synthetic construct
MNKQRGTFSEVSLAQDPKRQQRKPKGNKSSISGTEQEIFQVELNLQNPSLNHQGIDKIYDCQGLLPPPEK

SEQ ID NO: 95 - protein, 47 aa, synthetic construct
CWRKIKQSCEEENAGAEDQDGEGEGSKTALQPLKHSDSKEDDGQEIA

SEQ ID NO: 96 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 97 - DNA, 144 bp, synthetic construct
  source 1..144: mol_type = other DNA
caacgaaggaaatatagatcaaacaaaggagaaagtcctgtggagcctgcagagccttgtcattacagctgccccagggaggaggagggcagcaccatccccatccaggaggattaccgaaaaccggagcctgcctgctccccc

SEQ ID NO: 98 - protein, 123 aa, synthetic construct
AGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCC

SEQ ID NO: 99 - protein, 126 aa, synthetic construct
AAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTG

SEQ ID NO: 100 - protein, 126 aa, synthetic construct
GCCCTGTACCTGCTCCGGAGGGACCAGAGGCTGCCCCCCGATGCCCACAAGCCCCCTGGGGGAGGCAGTTTCCGGACCCCCATCCAAGAGGAGCAGGCCGACGCCCACTCCACCCTGGCCAAGATC

SEQ ID NO: 101 - DNA, 567 bp, synthetic construct
  source 1..567: mol_type = other DNA
tgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaag

SEQ ID NO: 102 - DNA, 186 bp, synthetic construct
  source 1..186: mol_type = other DNA
aaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacag

SEQ ID NO: 103 - DNA, 138 bp, synthetic construct
  source 1..138: mol_type = other DNA
aaggctctgatccacctgagcgacctccgggagtacaggcgctttgagaaggagaagctcaagtcccagtggaacaatgataatccccttttcaagagcgccaccacgacggtcatgaaccccaagtttgctgagagt

SEQ ID NO: 104 - DNA, 114 bp, synthetic construct
  source 1..114: mol_type = other DNA
tgttggcttacaaaaaagaagtattcatccagtgtgcacgaccctaacggtgaatacatgttcatgagagcagtgaacacagccaaaaaatctagactcacagatgtgacccta

SEQ ID NO: 105 - DNA, 348 bp, synthetic construct
  source 1..348: mol_type = other DNA
aaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaat

SEQ ID NO: 106 - DNA, 117 bp, synthetic construct
  source 1..117: mol_type = other DNA
aggacacagataaagaaactgtgctcgtggcgggataagaattcggcggcatgtgtggtgtacgaggacatgtcgcacagccgctgcaacacgctgtcctcccccaaccagtaccag

SEQ ID NO: 107 - DNA, 111 bp, synthetic construct
  source 1..111: mol_type = other DNA
atggaggagagtgtcgtacggccctcagtgtttgtggtggatggacagaccgacatcccattcacgaggctgggacgaagccaccggagacagtcgtgcagtgtggcccgg

SEQ ID NO: 108 - DNA, 210 bp, synthetic construct
  source 1..210: mol_type = other DNA
atgaataaacaaagaggaaccttctcagaagtgagtctggcccaggacccaaagcggcagcaaaggaaacctaaaggcaataaaagctccatttcaggaaccgaacaggaaatattccaagtagaattaaatcttcaaaatccttccctgaatcatcaagggattgataaaatatatgactgccaaggtttactgccacctccagagaag

SEQ ID NO: 109 - DNA, 141 bp, synthetic construct
  source 1..141: mol_type = other DNA
tgctggagaaagatcaaacagagctgtgaggaggagaatgcaggagctgaggaccaggatggggagggagaaggctccaagacagccctgcagcctctgaaacactctgacagcaaagaagatgatggacaagaaatagcc

SEQ ID NO: 110 - DNA, 339 bp, synthetic construct
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

(19) United States
(12) Patent Application Publication
(10) Pub. No.: US 2026/0234216 A1
(43) Pub. Date: Aug. 13, 2026

(54) UNIVERSAL ANTIBODY RECEPTORS, GUAR-T CELLS AND THERAPEUTIC USE THEREOF

(71) Applicant: YALE UNIVERSITY, New Haven, CT (US)

(72) Inventors: Sidi CHEN, New Haven, CT (US); Ping REN, New Haven, CT (US); Meizhu BAI, New Haven, CT (US)

(21) Appl. No.: 19/470,808

(22) PCT Filed: Apr. 1, 2024
(86) PCT No.: PCT/US2024/022496

§ 371 Date: Sep. 30, 2025

Related U.S. Application Data
(60) Provisional application No. 63/493,266, filed on Mar. 30, 2023.

(51) Int. Cl. C07K 14/705; A61K 35/17; A61K 40/11; A61K 40/32; A61P 35/00; C07K 16/28; C12N 5/0783

(52) U.S. Cl. CPC C07K 14/7051; A61K 35/17; A61K 40/11; A61K 40/32; A61P 35/00; C07K 14/70521; C07K 16/28; C12N 5/0636; A61K 2239/27; C07K 2317/565; C07K 2319/30; C12N 2510/00

(57) ABSTRACT

Compositions and methods for improved ACT are provided. Chimeric γδTCR cells including gamma-delta TCRs fused with an immunoglobulin antigen-binding domain targeting the Fc of human monoclonal antibodies (hmAb) are described. Compositions of chimeric γδTCR cells together with hmAbs that target antigen associated with a disease or disorder are described for selective ACT directed against the disease or disorder. The chimeric γδTCRs include removable masking domains that prevents non-specific T cell killing and reduce toxicity in vivo. Typically, the masking domain is removed by protease present in the TME. The compositions and methods provide enhanced ACT for cancer, auto-immune disease and other disease and disorders. Also disclosed are hmABs, genetically modified cells and pharmaceutical compositions and methods of use thereof for treating subjects having diseases or disorders.

### CROSS-REFERENCE TO RELATED APPLICATIONS

This application claims the benefit of and priority to U.S. Application No. 63/493,266 filed Mar. 30, 2023, the contents of which are incorporated by reference herein in their entirety.

### STATEMENT REGARDING FEDERALLY SPONSORED RESEARCH

This invention was made with government support under CA238295, CA231112 awarded by National Institutes of Health and W81XWH-20-1-0072 awarded by the Department of Defense. The government has certain rights in the invention.

### FIELD OF THE INVENTION

The invention is generally related to the fields of immunotherapy, and more particularly to variant chimeric antigen receptor (CAR) T cells that incorporate components of a gamma delta TCR together with a universal antibody receptor and methods of making and using thereof.

### REFERENCE TO THE SEQUENCE LISTING

The Sequence Listing XML submitted as a file named “YU_8621_PRO_ST26.xml”, and having a size of 526,381 bytes is hereby incorporated by reference pursuant to 37 C.F.R. § 1.834(c)(1).

### BACKGROUND OF THE INVENTION

Chimeric antigen receptor (CAR)-T cell therapy is a new treatment paradigm that redirects genetically engineered T cells to specifically recognize and lyse oncogenic cells in an MHC-independent manner. Many CAR-T cell candidates have achieved astonishing clinical results in hematological malignancies. It is worth noting that six CAR-T cell-based products have been approved by the United States Food and Drug Administration (US FDA) since 2017. However, unfavorable efficacy, safety issues, exhaustion, limited infiltration, and poor persistence are among the current major obstacles that hinder the clinical success of CAR-T therapy in refractory or resistant hematological malignancies, as well as solid tumors. Furthermore, current FDA-approved CAR-T cells are personalized (autologous) and restricted with single antigen-specific CARs, making the treatment more vulnerable to antigen loss. A number of studies have observed that antigen loss-associated tumor relapse remains a major challenge for maintaining long-term remission in a substantial fraction of CAR-T administered patients, which makes treatments of post-CAR relapse more difficult. Lastly, ex vivo manufacturing of a new cycle of CAR-T cells remains time-consuming and costly, during which the malignancy outgrowth may result in the deterioration of the disease and make patients no longer eligible for infusion of CAR-T cells. Thus, the development of more potent, adaptable and time/cost-effective T cell therapies is needed.

Along with the rapid development of adoptive cell therapies, a panel of CAR recognition combinations has been designed and investigated in different tumor indications. However, antigen heterogeneity is a barrier that limits CAR-T cells for tumor eradication, especially in solid tumors. In contrast to other multi-antigen-targeted CAR-T cell therapies, switchable universal CAR-T cells have shown superior performance. In principle, the receptors of switchable universal CAR-T cells do not directly recognize target antigens, but instead allow adaptable binding switch molecules to provide more flexibility in antigen recognition and controllability, where the activation of switchable universal CAR-T cells can be efficiently regulated by the presence of switch molecules. Various clinically approved molecules include human IgG1 antibodies, single chain variable fragment (scFv), Fab, VHH, peptides, and hapten tags that can be served as synapses and titrated in switchable universal CAR-T therapies.

Although switchable universal CAR-T can logically target any antigen for specificity, CAR-T cell early exhaustion remains a pivotal hurdle. CAR-mediated tonic signaling is associated with premature dysfunction and early T cell exhaustion, in canonical αβ-T cell-based CAR-Ts. Several functional and transcriptional studies revealed that high levels of CAR surface expression can spontaneously aggregate or cluster, and constitutively elicit different levels of tonic signaling to autoactivate CAR-T cells in an antigen-independent manner, which results in CAR-T exhaustion and thereby impairs anti-tumor function and clinical responses. In contrast to canonical αβ-T cell-based CAR-Ts, T cell receptor (TCR)-engineered T (TCR-T) cells have been shown to have hierarchical TCR-mediated tonic signaling outcomes in a stimulation-dependent manner, with reduced undesired effect in the absence of antigen stimulation. Furthermore, TCR-T cell therapies also exhibited encouraging anti-tumor efficacy with long-term persistence in certain cancer types. However, the broader application of TCR-T is still restrained by lack of targetable tumor-specific or tumor-associated antigens.

In addition, manufacture of allogeneic T cells has been widely discussed and considered as an appealing solution that helps to resolve many of the problems plaguing current CAR-T cell therapy, especially in graft-versus-host-disease (GvHD) and host allorejection. Several studies have revealed mutation in TRAC/TRBC locus that led to loss of αβ TCR on T cell surface, and further minimized the responses of graft-versus-host-disease (GVHD). Moreover, genetic ablation of β-2 Microglobulin (B2M) and Class II major histocompatibility complex transactivator (CIITA) could largely abolish the expression of HLA class I and HLA class II molecules, respectively, reducing immunogenicity of those engineered allogeneic T cells and enhancing outcomes of allogeneic T cell therapy. Allogeneic T cells can be derived from healthy donors and produced under highly standardized procedures, which makes this type of adoptive cell therapy an “off-the-shelf” product for affordable and readily-available cell-based cancer immunotherapy.

CAR-T cell therapy has demonstrated clinical success in the treatment of certain hematological malignancies. To date, six CAR-T cell products currently approved for clinical use by FDA, namely CD19-targeting KMYRIAH®, YESCARTA®, TECARTUS®, and BREYANZI®, and BCMA-targeting ABECMA®, and CARVIKTY®.

Nevertheless, high rates of therapeutic resistance and post-treatment tumor recurrence remain the primary limitations that restrain CAR-T cell use in broad clinical applications. Statistics showed that nearly half of pre-B cell ALL patients experience tumor relapse within 12 months of receiving anti-CD19 or anti-CD22 CAR-T cells. Tumor recurrence is also being detected even in dual-targeted CAR-T treatments, indicating that tumor antigen loss is a major obstacle to current CAR-T therapy.

To overcome the limitation of tumor antigen loss, several modular CAR-T cell platforms with universal adaptors are being evaluated. Universal modular CAR-T cells can logically target multiple antigens by administration of different CAR-adaptor molecules, and control the potential toxicity or undesired side effects of CAR-T by stopping the administration of CAR-adaptor molecules. Universal CAR-T cells can be expanded without additional engineering, reducing cost and offering a personal “off-the-shelf” T cell product for patients against tumor relapse.

However, like other single antigen-specific CAR-T cells, universal CAR-T cells are commonly used in similar downstream signaling cascades of CAR activation and are generated by lentiviral/retroviral-associated methods, resulting in high CAR expression levels on T cell surfaces. Excessive CARs can cause spontaneous CAR clustering in an antigen-independent manner, leading to tonic signaling and early exhaustion of CAR-T cells. TCR-T cells use natural TCR signaling, which is less prone to tonic signaling and less prone to exhaustion, but their applications are still restricted to MHC proteins with certain HLA alleles or the availability of antigen-specific TCRs.

Therefore, there is a need for enhanced artificial T cells with new types of chimeric immunoreceptors that are engineered for broadly effective and adaptable therapeutic strategies.

Therefore, it is an object of the invention to provide new types of artificial T cells with chimeric immunoreceptors having improved anti-tumor efficacy and methods thereof for treating proliferative diseases.

It is another object of the invention to provide new types of artificial T cells with chimeric immunoreceptors capable of homing to and selectively binding therapeutic antibodies.

It is a further object of the invention to provide therapeutic antibodies that exhibit effective anti-tumor activity against a wide range of cancer types.

It is yet another object of the invention to provide systems for enhancing therapeutic activity of engineered cells of various immune cell types.

### SUMMARY OF THE INVENTION

Harnessing a combination of chimeric gamma-delta T-cell receptor (γδ-TCR) and antibody recognition mechanisms, a universal, switchable cell therapy platform has been developed. This platform, termed Gamma delta TCR signaling directed Universal Antibody Receptor (GUAR) T cell therapy, engineers TCR γ/δ chain constant regions with variable regions of an anti-hIgG1-Fc antibody heavy and light chains, respectively. GUAR-expressing T cells (GUAR-Ts) recognize the Fc-portion of therapeutic antibodies, which facilitates the selective targeting of different antigens on-demand with different therapeutic monoclonal antibodies, and maintain potent and sensitive signaling capacity via γδTCR signaling. Stable knock-in GUAR-T cells showed potent antibody-dependent activity and in vivo anti-tumor efficacy. Given the repertoire of rapidly increasing therapeutic antibodies, GUAR-T opens immense potential as a highly adaptive and universal cell therapy.

Thus, compositions and methods of use of Gamma delta TCR signaling directed Universal Antibody Receptor (GUAR) constructs and engineered T cells formed therewith have been developed. The disclosed compositions and methods are especially applicable to T cell therapy.

Compositions including a chimeric gamma delta T-cell receptor (γδTCR) are provided. The chimeric gamma delta T-cell receptor (γδTCR) can include constant and variable immunoglobulin domains, wherein the constant immunoglobulin domains include a gamma TCR constant domain and a delta TCR constant domain, and wherein the variable immunoglobulin domains include an immunoglobulin antigen binding variable light domain, and an immunoglobulin antigen binding variable heavy domain, wherein the antigen binding domains specifically bind constant domain(s) of human immunoglobulin IgG, are provided.

In some forms, the chimeric γδTCR includes the amino acid sequence of any one or more of 1-2, optionally light and heavy chain variable regions including the CDRs of SEQ ID NOS:3 and 4, or SEQ ID NOS:3 and/or 4; or functional fragments or variant thereof having at least 70% sequence identity thereto. In some forms, the chimeric γδTCR includes the amino acid sequence of any one or more of SEQ ID NOs:1-4, or a functional variant thereof having at least 75% sequence identity to any one or more of SEQ ID NOs:1-4. In some forms, the chimeric γδTCR includes the amino acid sequence of, such as all of any of SEQ ID NOs:1-4.

In some forms, the chimeric γδTCR includes a removable masking moiety that prevents the antigen binding domains from specifically binding to constant domain(s) of human immunoglobulin IgG. Exemplary masking moieties include, but are not limited to, coiled-coil peptides selected from CC2B, CC3, CC4, and CC5.

In certain forms, the masking moiety is a protease-cleavable masking moiety, for example, that is removed in the presence of a protease enzyme. An exemplary protease is a urokinase, for example, such as that present within the tumor microenvironment (TME). In some forms, the masking moiety includes LSGRSDNH (SEQ ID NO:5).

In some forms, the chimeric γδTCR includes one or more intracellular domain(s) of a costimulatory molecule selected from CD27, CD28, CD137, 0X40, IL2Rβ, ICOS, IL7Rα, CD30, CD40, CD3, LFA 1, ICOS, CD2, CD7, LIGHT, NKG2C, B7 H3, and ligands of CD83. In some forms, the intracellular domain of CD28 is contiguous with the delta TCR constant domain. In some forms, the intracellular domain of any one of 0X40, IL2Rβ, ICOS, or IL7Rα, is contiguous with the gamma TCR constant domain.

For example, in some forms, chimeric γδTCR includes one or more of SEQ ID NOs:83-96.

Nucleic acids encoding or expressing a chimeric γδTCR are also described, and vectors including the nucleic acid are also described. Cells including a chimeric γδTCR, or the nucleic acid encoding or expressing the chimeric γδTCR, or the vector including the nucleic acids. A genetically modified T-cell expressing a chimeric γδTCR is also provided. Typically, the genetically modified T-cell is activated upon binding of the variable antigen binding domains to the constant domain(s) of human immunoglobulin IgG. In some forms, the cell expressing the chimeric γδTCR includes one or more additional genetic modifications in a gene selected from the group including TRAC, TRBC, B2M and CIITA.

Populations of cells derived by expanding a genetically modified T-cell expressing the chimeric γδTCR is also provided.

Pharmaceutical compositions including a population of cells and a pharmaceutically acceptable buffer, carrier, diluent or excipient are also described. In some forms, the pharmaceutical composition further includes one or more clones of human monoclonal antibodies. In some forms, the one or more clones of human monoclonal antibodies includes antibodies that specifically bind to an antigen expressed on a cancer cell. Exemplary human monoclonal antibodies specifically bind to anti-GPRC5D antibody.

Methods of treating a subject having a disease, disorder, or condition including administering to the subject an effective amount of the pharmaceutical composition including population of cells derived by expanding a cell genetically modified T-cell expressing the chimeric γδTCR is also provided. A method of treating a subject having a disease, disorder, or condition associated with an elevated expression or specific expression of an antigen are provided. Typically, the methods include administering to the subject an effective amount of a pharmaceutical composition including a chimeric γδTCR, and a human monoclonal antibody that targets the antigen. In some forms, the monoclonal antibody targets an antigen expressed on a cancer cell. The monoclonal antibody can be administered to the patient before, or after the pharmaceutical composition including the chimeric γδTCR, or at the same time as the pharmaceutical composition including the chimeric γδTCR. In some forms, the monoclonal antibody is administered to the patient via the same or different route of administration as the pharmaceutical composition including the chimeric γδTCR. In some forms, the subject has cancer, or has been identified as being at increased risk of getting cancer. In some forms, the population of cells were isolated from or derived from the expansion of a cell obtained from the subject having the disease, disorder, or condition prior to the introduction to the cell. In other forms, the population of cells were isolated from, or derived from the expansion of a cell obtained from a healthy donor.

Kits including a cell expressing the chimeric γδTCR, and/or a nucleic acid encoding the chimeric γδTCR, and/or a vector including the nucleic acid, optionally further including a human monoclonal antibody are also provided.

Also described is a monoclonal antibody that specifically binds to GPRC5D, including a light chain variable region including the CDRs of SEQ ID NO:19 (optionally SEQ ID NOS:24-26) optionally the light chain variable region of SEQ ID NO:19 or a variant thereof with at least 70% sequence identity thereto; a heavy chain variable region including the CDRs of SEQ ID NO:17 (optionally SEQ ID NOS:21-23) optionally the heavy chain variable region of SEQ ID NO:17 or a variant thereof with at least 70% sequence identity thereto; or more preferably a combination thereof, or an antigen-binding fragment or variant thereof.

### BRIEF DESCRIPTION OF THE DRAWINGS

The accompanying drawings, which are incorporated in and constitute a part of this specification, illustrate several forms of the disclosed method and compositions and together with the description, explain the principles of the disclosed method and compositions.

FIG. 1A is a schematic diagram showing the modular GUAR-T cell therapy. The cross-linking of GUAR-T cells and target cells is mediated through human IgG1-based antibodies, which are capable of binding to an antigen of interest present in the target cells. FIG. 1B is a schematic diagram showing antibody discovery processes. Two Ig humanized mice were vaccinated via subcutaneous injection route with Freund's adjuvant-mixed proteins, then antibody candidates were identified through single B cell sequencing. FIG. 1C is a schematic diagram showing yeast surface display of reconstructed clonotypes from single B cell sequencing. FIG. 1D is a schematic diagram showing GUAR on lentiviral vector. FIG. 1E is a schematic diagram showing characterization of pairing GUAR receptor candidates by the mammalian surface display.

FIG. 2A is a schematic diagram showing the targeting strategy to generate GUAR-Clone2 knock-in at TRAC locus. FIG. 2B is a schematic diagram showing γδTCR-based universal chimeric antibody receptor. FIGS. 2C and 2D are graphs showing binding capacity of GUAR-T-Clone2 measured by titrating biotinylated human IgG1-Fc. GUAR-T-Clone2 cells were incubated with a biotinylated human IgG1-Fc, which was then detected using a PE-Streptavidin antibody. Percentage of antigen binding positive cells (FIG. 2C); MFI of antigen binding positive cells (FIG. 2D). FIG. 2E is a bar graph showing activation of Jurkat-TCRαβ-KO and GUAR-J-Clone2 cells upon antigen stimulation, as detected by CD69 expression. Jurkat-TCRαβ-KO and GUAR-J-Clone2 cells were stimulated with plate-bound human IgG1-Fc peptide (5 μg/ml) for 0 h or 5 h. FIG. 2F is a graph showing ERK phosphorylation of GUAR-J-Clone2 cells upon cross-linking. GUAR-J-Clone2 cells were incubated with soluble human IgG1-Fc biotin and then cross-linked by streptavidin. FIGS. 2G-2I are graphs showing ERK phosphorylation of GUAR-T-Clone2-expressing human CD3 T cells (FIG. 2G), CD4 T cells (FIG. 2H), and CD8 T cells (FIG. 2I) upon cross-linking. Human T cells were incubated with soluble human IgG1-Fc biotin and then cross-linked by streptavidin. FIGS. 2J-2L are graphs showing ZAP70 phosphorylation of GUAR-T-Clone2-expressing human CD3 T cells (FIG. 2J), CD4 T cells (FIG. 2K), and CD8 T cells (FIG. 2L) upon cross-linking.

FIG. 3A is a schematic diagram showing GUAR-T system targeting tumor antigen using a specific antibody. MM.1R cells over-expressing BCMA were co-cultured in vitro with GUAR-expressing CD3+ human primary T cells (GUAR-T cells). FIG. 3B is a bar graph showing cytotoxicity (% killing) of GUAR-T-Clone2 toward MM.1R-BCMA after 12-hour coculture with 5 μg/mL of anti-BCMA-IgG1 mAb at different E:T ratio as determined by luciferase assay; data shown as means±SD are from four independent replicates. Statistical analyses were performed based on unpaired t-test. FIG. 3C is a line graph showing cytotoxicity (% killing) of GUAR-T-Clone2 toward MM.1R-BCMA after 12-hour coculture, titrated with different anti-BCMA-IgG1 mAb concentrations at different E:T ratios as determined by luciferase assay; data shown as means±SD are from four independent replicates. Statistical analyses were performed based on two-way ANOVA. FIGS. 3D and 3E are line graphs showing effects of different anti-BCMA-IgG1 mAb concentrations on the IFNγ (FIG. 3D) and TNFα (FIG. 3E) production by primary human CD4+ and CD8+ T cells after coculture with MM.1R-BCMA cancer cells at an E:T ratio of 1:1; n=3. Statistical analyses were performed based on two-way ANOVA. FIG. 3F is a line graph showing effects of different anti-BCMA-IgG1 mAb concentrations on the CD107a expression in primary human CD4+ and CD8+ T cells after coculture with MM.1R-BCMA cancer cells at an E:T ratio of 1:1; n=3. Statistical analyses were performed based on two-way ANOVA. FIG. 3G is a schematic diagram showing GUAR-T system targeting tumor antigen using a specific antibody. MM.1R-BCMA cells that also express GPRC5D were co-cultured in vitro with GUAR-T cells. FIGS. 3H and 3I are bar graphs showing cytotoxicity (% killing) of GUAR-T-Clone2 toward MM.1R-BCMA after 24 hours (FIG. 3H) and 72 hours (FIG. 3I) respectively coculture with different anti-GPRC5D-IgG1 mAb concentrations at an E:T ratios of 1:1 as determined by luciferase assay; data shown as means±SD are from four independent replicates. Statistical analyses were performed based on two-way ANOVA. FIG. 3J is a schematic diagram showing GUAR-T system targeting tumor antigen using a specific antibody. SKOV3 cells expressing HER2 were co-cultured in vitro with GUAR-T cells. FIG. 3K is a bar graph showing cytotoxicity of GUAR-T-Clone2 toward SKOV3 after 12-hour coculture with different anti-HER2-IgG1 mAb concentrations at an E:T ratio of 1:1 as determined by luciferase assay; data shown as means±SD are from four independent replicates. Statistical analyses were performed based on unpaired t-test. FIG. 3L is a bar graph showing cytotoxicity of GUAR-T-Clone2 toward SKOV3 after 24-hour coculture with 0.01 μg/mL of anti-HER2-IgG1 mAb at different E:T ratios as determined by luciferase assay; data shown as means±SD are from four independent replicates. Statistical analyses were performed based on unpaired t-test. FIGS. 3M, 3N, and 3O are bar graphs showing percentage of cytokine release of GUAR-T-Clone2 upon 6 h stimulation with cancer and specific antibody (0.1 μg/mL) at an E:T ratio of 1:1. IL2 (FIG. 3M), IFNγ (FIG. 3N), TNAα (FIG. 3P); data shown as means±SD are from three independent replicates. Statistical analyses were performed based on an unpaired t-test. FIG. 3P is a bar graph showing percentage CD107a expression of GUAR-T-Clone2 upon 6 h stimulation with SKOV3 and anti-HER2-IgG1 mAb (0.1 μg/mL) at an E:T ratio of 1:1; data shown as means±SD are from three independent replicates. Statistical analyses were performed based on unpaired t-test.

FIG. 4A is a schematic diagram showing experimental timeline of GUAR-T-Clone2 in vivo antitumor assay in mice bearing SKOV3 solid tumor xenografts. FIG. 4B is a line graph showing tumor burden quantified as the total flux (photons/s) from the luciferase activity of each mouse using IVIS imaging. Arrow indicates injection of engineered CD3+ T cells and highlighted region indicates injection of antibody [every day at 3 mg/kg for 12 days]. Statistical analysis was performed based on two-way ANOVA. FIG. 4C is a survival curve of SKOV3 inoculated NSG mice treated with DPBS, GUAR-T-Clone2, anti-HER2-IgG1 mAb, or GUAR-T-Clone2 with anti-HER2-IgG1 mAb. Statistical analyses were performed based on Log-rank (Mantel-Cox) test. FIG. 4D is a graph showing in vivo IFNγ cytokine level at 24 h or 48 h after PBS, GUAR-T-Clone2, anti-HER2-IgG1 mAb, or GUAR-T-Clone2 with anti-HER2-IgG1 mAb infusion in SKOV3 inoculated NSG model. Data shown as means±SD are from at least three independent replicates. Statistical analyses were performed based on unpaired t-test.

FIG. 5A is a schematic showing the domain arrangement of hIgG1-Fc WT protein and its artificial mutants. Alanine mutations were labeled. FIG. 5B is a SDS-PAGE analysis of purified hIgG1-Fc WT proteins and mutants under nonreducing and reducing (10 mM DTT) conditions. Four micrograms of each purified protein were analyzed using a Novex WedgeWell 4-20% (wt/vol) Tris-Glycine gel.

FIGS. 6A-6B are schematic illustrations of masked GUAR in healthy tissues (no signaling; FIG. 6A) and in tumor (FIG. 6B), with the mask being cleaved by various tumor-associated proteases. FIG. 6C is a graph showing histograms of the surface expression of different masked GUAR variants on Jurkat cells. FIG. 6D is a graph showing histograms of the activation marker CD69 expression in Jurkat cells transduced to express CAR or non-masked GUAR or masked-CC3 GUAR in the absence of antigen. FIG. 6E is a line graph showing percent hIgG1-Fc binding of non-masked GUART-T cells and masked-CC3 GUAR-T cells based on analysis using flow cytometry in the absence/presence of proteases at different concentrations. Cleaved comparators were generated using recombinant uPA before the binding assay. Experiments with three independent donors were conducted, with similar results.

FIGS. 7A-7C are bar graphs showing gene editing efficiency of different sgRNAs targeting TRAC, B2M, and CIITA, respectively by analyzing surface expression of CD3 (FIG. 7A), HLA-A/B/C (FIG. 7B), and HLA-DP/DQ/DR (FIG. 7C) by flow cytometry. FIG. 7D is a schematic diagram showing Cas9 RNP mixture (including sgRNAs targeting TRAC, TRBC, B2M, CIITA) electroporation combined with AAV-delivered HDR template, enabling knock-out of TRAC/TRBC/B2M/CIITA as well as knock-in of GUAR-Clone2 into TRAC locus in human primary ap T cells. FIG. 7E is a schematic diagram showing the targeting strategy to generate GUAR-Clone2 knock-in at TRDC locus.

FIG. 8A shows SDS-PAGE analysis of purified human IgG1-Fc protein under nonreducing and reducing conditions. FIG. 8B shows protein activity was determined by ELISA. FIGS. 8C-8D are line graphs showing antibody titer in serum samples in mouse L (FIG. 8D) and mouse LR (FIG. 8C). The vaccinated mice were defined as Mouse L (left ear clipping), and Mouse LR (both ears clipping) based on ear clipping. All serum samples were serially 10-fold dilution from 1:200 and assayed by a direct-coating ELISA with the purified hIgG-Fc protein-coated plate. Error bars represent the mean±SEM of triplicates with individual data points in plots. FIG. 8E shows heatmap for non-stochastic paired BCR repertoire.

FIG. 9 shows surveyor assay to detect TRBC1 and TRBC2 loci editing efficacy in GUAR-T-Clone2 cells. For TRBC1 locus, two sets of primers were designed and used for the assay.

FIGS. 10A-10C are graphs showing BCMA and GPRC5D are prognostic factors in multiple myeloma (MM) patients. High expression of BCMA (FIG. 10A) and GPRC5D (FIG. 10B) predicts unfavorable survival on 767 newly diagnosed MM samples in MMRF datasets; the combination of BCMA and GPRC5D (FIG. 10C) could stratify patients with the shortest 2-year overall survival (OS) time, while BCMA^{high} only (yellow line) and GPRCD5^{high} only (green line) have better outcomes compared with BCMA^{high}/GPRC5D^{high} patients. FIG. 10D is a scatter plot of BCMA and GPRC5D mRNA expression in MMRF datasets (MMRF: Multiple Myeloma Research Foundation CoMMpass study; P values were measured with log-rank test for survival analysis). FIG. 10E shows BCMA surface expression on MM.1R-BCMA cells quantified by flow cytometry. FIG. 10F shows cytotoxicity of GUAR-T-Clone2 toward MM.1R-BCMA after 12 h or 24 h coculture with different anti-BCMA-IgG1 mAb concentrations at an E:T ratio of 1:2 as determined by luciferase assay; data shown as means±SD are from four independent experiments. Statistical analyses were performed based on two-way ANOVA. FIGS. 10G-10H are bar graphs showing percentage of cytokine release of GUAR-T-Clone2 generated from 3 different donors upon 6 h stimulation with MM.1R-BCMA cells and anti-BCMA-IgG1 mAb (0.1 μg/mL) at an E:T ratio of 1:1. IFNγ (FIG. 10G), TNFα (FIG. 10H); data shown as means±SD are from three independent replicates. Statistical analyses were performed based on an unpaired t-tests. FIG. 10I is a bar graph showing percentage of CD107a expression of GUAR-T-Clone2 generated from 3 different donors upon 6 h stimulation with MM.1R-BCMA cells and anti-BCMA-IgG1 mAb (0.1 μg/mL) at an E:T ratio of 1:1. Data shown as means±SD are from three independent replicates. Statistical analyses were performed based on an unpaired t-tests. FIGS. 10J-10O are representative flow cytometry and statistical analysis showing inhibitory receptor (PD1, LAG3, TIM3) expression on GUAR-T-Clone2 cell upon stimulation with MM.1R-BCMA cells and anti-BCMA-IgG1 mAb (0.1 μg/mL) at an E:T ratio of 1:1. CD4-based GUAR-T-Clone2 cell population (FIGS. 10J, 10K, and 10L). CD8-based GUAR-T-Clone2 cell population (FIGS. 10M, 10N, and 10P). Data shown as means±SD are from three independent replicates. FIG. 10P is a graph showing HER2 surface expression on SKOV3 cells quantified by flow cytometry. FIGS. 10Q-10R are bar graphs showing percentage of cytokine release of GUAR-T-Clone2 generated from 3 different donors upon 6 h stimulation with SKOV3 and anti-HER2-IgG1 mAb (0.1 μg/mL) at an E:T ratio of 1:1. IFNγ (FIG. 10R), TNFα (FIG. 10S); data shown as means±SD are from three independent replicates. Statistical analyses were performed based on an unpaired t-test. FIG. 10S is a bar graph showing percentage of CD107a expression of GUAR-T-Clone2 generated from 3 different donors upon 6 h stimulation with SKOV3 and anti-HER2-IgG1 mAb (0.1 μg/mL) at an E:T ratio of 1:1. Data shown as means±SD are from three independent replicates. Statistical analyses were performed based on an unpaired t-test.

FIG. 11A is a schematic diagram showing experimental timeline of GUAR-T-Clone2 in vivo antitumor assay in mice bearing SKOV3 xenograft tumors. Spleen and orbital blood were collected 14 days after GUAR-T-Clone2 infusion. FIG. 11B is a schematic diagram of the targeting strategy to generate huEGFRt knock-in at TRAC locus. FIG. 11C is a bar graph showing the ratio of CD4 over CD8 population of infused GUAR-T cells in SKOV3 inoculated mice spleen. Data shown as means±SD are from three independent mice. Statistical analyses were performed based on an unpaired t-test. FIGS. 11D-11E are bar graphs showing the percentage of effector memory (FIG. 11D) and central memory (FIG. 11E) population of infused GUAR-T cells in SKOV3 inoculated mice spleen. Data shown as means±SD are from three independent mice. Statistical analyses were performed based on an unpaired t-test.

FIG. 12A is a schematic diagram showing the targeting strategy to generate modularized GUAR-Clone2 knock-in at TRAC locus. CD28 intracellular domain was fixed to the C-terminal of the TCR δ chain, while the intracellular domain of OX40, ICOS, IL7Rα, or ΔIL2Rβ was integrated to the C-terminal of TCR γ chain respectively. FIG. 12B is a schematic diagram showing γδTCR-based universal chimeric antibody receptor with CD28 intracellular domain to the C-terminal of the TCR δ chain, as well as a series of constructs where the intracellular domain of OX40, ICOS, IL7Rα, or ΔIL2Rβ is fused to C-terminal of TCR γ chain, respectively. FIG. 12C is a bar graph showing cytotoxicity of modularized GUAR-T-Clone2 toward SKOV3 tumor cells was measured by luciferase assay after 24 h co-culture with 0.01 μg/mL of anti-HER2-IgG1 mAb at an E:T ratio of 1:2. Data shown as means±SD are from four independent replicates. Statistical analyses were performed based on an unpaired t-tests. FIG. 12D is a schematic diagram showing the targeting strategy to generate GUAR-Clone2 knock-in at TRAC locus which utilizes TRAC endogenous promoter to transcriptionally regulate GUAR-Clone2 expression. FIGS. 12E-12H are bar graphs showing percentage of cytokine release of EFS- or TRAC-driven GUAR-T-Clone2 cells upon 6 h stimulation with SKOV3 tumor cells and 0.1 μg/mL of anti-HER2-IgG1 mAb at an E:T ratio of 1:1. IFNγ (FIG. 12E) and TNFα (FIG. 12F) in CD4-based GUAR-T cell population, and IFNγ (FIG. 12G) TNFα (FIG. 12H) in CD8-based GUAR-T cell population. Data shown as means±SD are from three independent replicates. Statistical analyses were performed based on an unpaired t-test. FIGS. 12I-12J are bar graphs showing percentage of CD107a expression in EFS- or TRAC-driven GUAR-T-Clone2 cells upon 6 h stimulation with SKOV3 tumor cells and 0.1 μg/mL of anti-HER2-IgG1 mAb at an E:T ratio of 1:1. CD4-based GUAR-T cell population (FIG. 12I), CD8-based GUAR-T cell population (FIG. 12J). Data shown as means±SD are from three independent replicates. Statistical analyses were performed based on an unpaired t-tests.

FIGS. 13A-13B are schematic diagrams of masked-CC3 GUAR-J (5) (FIG. 13A) and masked-CC2B GUAR-J (5) (FIG. 13B) constructs.

FIGS. 14A-14E are histograms of the activation marker CD69 expression in Jurkat cells transduced to express CAR or non-masked GUAR or masked-CC3 GUAR in the absence of antigen.

### DETAILED DESCRIPTION OF THE INVENTION

The disclosed method and compositions can be understood more readily by reference to the following detailed description of forms and the Examples included therein and to the Figures and their previous and following description.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### I. Definitions

“Introduce” in the context of genome modification refers to bringing in to contact. For example, to introduce a gene editing composition to a cell is to provide contact between the cell and the composition. The term encompasses penetration of the contacted composition to the interior of the cell by any suitable means, e.g., via transfection, electroporation, transduction, gene gun, nanoparticle delivery, etc.

“Homologous” refers to the sequence similarity or sequence identity between two polypeptides or between two nucleic acid molecules. When a position in both of two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or are homologous, then the two sequences are 60% homologous. By way of example, the DNA sequences ATTGCC and TATGGC share 50% homology. Generally, a comparison is made when two sequences are aligned to give maximum homology.

“Endogenous” refers to any material from or produced inside an organism, cell, tissue or system. “Exogenous” refers to any material introduced from or produced outside an organism, cell, tissue or system.

The term “antigen” as used herein is defined as a molecule capable of being bound by an antibody or T-cell receptor. An antigen can additionally be capable of provoking an immune response. This immune response can involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which includes a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the disclosed compositions and methods includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid. In the context of cancer, “antigen” refers to an antigenic substance that is produced in a tumor cell, which can therefore trigger an immune response in the host. These cancer antigens can be useful as markers for identifying a tumor cell, which could be a potential candidate/target during treatment or therapy. There are several types of cancer or tumor antigens. There are tumor specific antigens (TSA) which are present only on tumor cells and not on healthy cells, as well as tumor associated antigens (TAA) which are present in tumor cells and on some normal cells. In some forms, the chimeric antigen receptors are specific for tumor specific antigens. In some forms, the chimeric antigen receptors are specific for tumor associated antigens. In some forms, the chimeric antigen receptors are specific both for one or more tumor specific antigens and one or more tumor associated antigens.

“Bi-specific chimeric antigen receptor” refers to a CAR that includes two domains, wherein the first domain is specific for a first ligand/antigen/target, and wherein the second domain is specific for a second ligand/antigen/target. In some forms, the ligand is a B-cell specific protein, a tumor-specific ligand/antigen/target, a tumor associated ligand/antigen/target, or combinations thereof. A bispecific CAR is specific to two different antigens. A multi-specific or multivalent CAR is specific to more than one different antigen, e.g., 2, 3, 4, 5, or more. In some forms, a multi-specific or multivalent CAR targets and/or binds three or more different antigens.

“Encoding” or “encode” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “locus” is the specific physical location of a DNA sequence (e.g., of a gene) on a chromosome. It is understood that a locus of interest can not only qualify a nucleic acid sequence that exists in the main body of genetic material (i.e., in a chromosome) of a cell but also a portion of genetic material that can exist independently to said main body of genetic material such as plasmids, episomes, virus, transposons or in organelles such as mitochondria as non-limiting examples.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

In the context of cells, the term “isolated” also refers to a cell altered or removed from its natural state. That is, the cell is in an environment different from that in which the cell naturally occurs, e.g., separated from its natural milieu such as by concentrating to a concentration at which it is not found in nature. “Isolated cell” is meant to include cells that are within samples that are substantially enriched for the cell of interest and/or in which the cell of interest is partially or substantially purified.

As used herein, “transformed,” “transduced,” and “transfected” encompass the introduction of a nucleic acid or other material into a cell by one of a number of techniques known in the art.

A “vector” is a composition of matter which includes an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” encompasses an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus (AAV) vectors, retroviral vectors, and the like.

“Tumor burden” or “tumor load” as used herein, refers to the number of cancer cells, the size or mass of a tumor, or the total amount of tumor/cancer in a particular region of a subject. Methods of determining tumor burden for different contexts are known in the art, and the appropriate method can be selected by the skilled person. For example, in some forms tumor burden can be assessed using guidelines provided in the Response Evaluation Criteria in Solid Tumors (RECIST).

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

The term “inhibit” or other forms of the word such as “inhibiting” or “inhibition” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” can also mean to hinder or restrain the synthesis, expression or function of a protein relative to a standard or control. Inhibition can include, but is not limited to, the complete ablation of the activity, response, condition, or disease. “Inhibits” can also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels. For example, “inhibits expression” means hindering, interfering with or restraining the expression and/or activity of the gene/gene product pathway relative to a standard or a control.

“Treatment” or “treating” means to administer a composition to a subject or a system with an undesired condition (e.g., cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition (e.g., cancer). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the term “polypeptides” includes proteins and functional fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “functional fragment” as used herein is a fragment of a full-length protein retaining one or more function properties of the full-length protein.

As used herein, the terms “variant” or “active variant” refers to a polypeptide or polynucleotide that differs from a reference polypeptide or polynucleotide, but retains essential properties (e.g., functional or biological activity). A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological or functional activity, certain amino acid sequence substitutions can be made in a polypeptide sequence and nevertheless obtain a polypeptide with like properties (e.g., functional or biological activity).

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological forms. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Forms of this disclosure thus contemplate functional or biological equivalents of a polypeptide as set forth above. In particular, forms of the polypeptides can include variants having about 50%, 60%, 70%, 80%, 90%, and 95% sequence identity to the polypeptide of interest.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. Compositions

A platform for universal, switchable cell therapy, harnessing a combination of chimeric γδTCR and a customized specific antibody recognition mechanism has been established. This platform, termed Gamma delta TCR signaling directed Universal Antibody Receptor (GUAR) T cell therapy, engineers TCR γ/δ chain constant regions with variable regions of a custom anti-hIgG1-Fc antibody heavy and light chains. The antibody receptor domains in the GUAR recognize the “constant” immunoglobulin (Fc) domain of IgG antibodies or another isotype. Accordingly, GUAR-expressing T cells (GUAR-Ts) can recognize the Fc-portion of therapeutic antibodies to facilitate selective targeting of different antigens on-demand, and maintain potent and sensitive signaling capacity of the native TCR complex mediated by γδTCR signaling. GUAR-T's γδTCR signaling utilizes the endogenous/native signal transduction that minimizes tonic signaling to avoid T cell exhaustion and dysfunction. In contrast to other CAR-T designs, GUAR-T uses antibody receptor, which does not require development of additional binders for each antigen, but instead can utilize existing therapeutic antibodies. Given that there are over 100 FDA-approved therapeutic antibodies, with many more in various stages of preclinical and clinical development, GUAR-T opens immense potential for their utilization via highly adaptive and universal cell therapy.

As demonstrated in the Examples, GUAR-Ts facilitate selective antigen targeting with multiple independent therapeutic monoclonal antibodies (mAbs) (exemplified with anti-HER2, anti-BCMA and anti-GPRC5D mAbs). The stable TRAC knock-in GUAR-T cells exhibited potent antibody-dependent functional activity such as T cell activation, cytolysis, and in vivo anti-tumor efficacy.

To enhance GUAR-T safety features to prevent on-target-off-tumor activity, AlphaFold2-Multimer guided structural engineering was utilized for precise design of masks to curb the unwanted activity of GUAR and allow it to be activated only in the presence of tumor microenvironment specific proteases (e.g., uPA). Further, endogenous gene editing on genes such as TRAC, TRBC, B2M and CIITA allows development of allogeneic GUAR-T for “off-the-shelf” cell therapy.

The described GUAR molecules and GUAR-T cells provide a new fundamental core structure for “chimeric antibody receptors”. GUAR-T uses a universal antibody receptor that makes adaptation of new binders as simple as adding the therapeutic antibody, and the relatively more complex cellular engineering part produces the universally adaptable T cells that can fit all types of Ig-based, e.g., IgG1-based, binders that represent majority of therapeutic antibodies. This is a “plug-and-play” type of cell therapy. In addition, GUAR-T harnesses the γδTCR signaling that overcomes tonic signaling issues that CAR-T cells commonly face. Utilization of γδTCR also avoids the competition of endogenous beta chain of TCR in TRAC-targeted engineering. All the GUAR-T structure and therapeutic components (cells, γδTCR, anti-hIgG1, anti-HER2/BCMA/GPRC5D/etc.) can be fully human, i.e., developed from humanized mice.

Compositions of nucleic acids and polypeptides encoding GUAR molecules alone and also together with cofactors, such as the components of the CD3 complex are provided. Recombinant constructs including nucleic acids expressing or encoding the GUAR polypeptides and fusion proteins including masking moieties and/or intracellular domains are also provided. Viral genomes including the recombinant constructs, recombinant viruses including the constructs, and recombinant cells formed thereof are also provided. The GUAR polypeptides, nucleic acids encoding the same, and delivery vehicles thereof and cells including them can optionally include one or more additional heterologous proteins, polypeptides or other amino acid sequences.

### A. Gamma Delta TCR Signaling Directed Universal Antibody Receptor (GUAR)

GUAR molecules, compositions encoding and/or including the same, and GUAR-T cells expressing these molecules at the cell surface are provided. For example, polypeptides including the engineered components of GUAR molecules are disclosed. As discussed in more detail below, the GUAR polypeptides typically include a chimeric TCR gamma subunit and a chimeric TCR delta subunit each including between 200 and 300 contiguous amino acids, including approximately 100 amino acids in each immunoglobulin domain. The chimeric TCR gamma subunit and a chimeric TCR delta subunit can also be referred to as arms of the GUAR that assemble to form the GUAR complex. The arms can be expressed together as a pre-protein e.g., that is cleaved during post-translation modification as discussed in more detail below, or as separate proteins. Thus, nucleic acids encoding individual arms as well as a single nucleic acid encoding both arms are each provided.

Typically, the GUAR is a membrane-bound, cell surface receptor having an extracellular domain, transmembrane domain, and intracellular cytoplasmic domain. As used herein, a “transmembrane domain” refers to any protein structure that is thermodynamically stable in a cell membrane, preferably a eukaryotic cell membrane. As used herein, the terms “extracellular domain” and “ectodomain” refer to any protein structure that is thermodynamically stable in outside of the cell membrane (i.e., in the extracellular space). As used herein, an “intracellular domain” refers to any protein structure that is thermodynamically stable in inside of the cell membrane (i.e., in the intracellular cytosol).

Typically, the GUAR molecule includes:
a. A gamma-delta T-cell receptor molecule, lacking the antigen binding (variable) domains;
b. Variable heavy and light immunoglobulin domains from an antigen binding domain(s) developed to bind an antibody constant region, e.g., a human IgG antibody constant region (Fc), in place of the γδTCR-antigen binding regions;
c. Optionally one or more removable masking moieties, configured to occlude or otherwise disrupt the constant region (e.g., IgG Fc)-binding activity of the GUAR and preclude undesired auto-immunity based on GUAR-T mediated killing by recognition of all human antibodies (e.g., IgG) in vivo; and
d. Optionally one or more intracellular domains from a co-stimulatory factor, configured to enforce or enhance the co-stimulator-based activation of GUAR T-cells. Each of these components is described in greater detail, below.

### 1. TCR Gamma and Delta Chimeric Subunits

Each GUAR can include a chimeric TCR gamma subunit and a chimeric TCR delta subunit, as well as the “native’ CD3 complex, including CD3 epsilon (×2), CD3 gamma and CD3 delta subunits. The chimeric subunits heterodimerize based on canonical dimerization of the native gamma-delta TCR and spontaneously fold/are exported to the cell surface following ribosomal translation.

Gamma delta T cells are a major player in cancer immunotherapy, despite the fact that therapeutic use of γδ T cell is limited. Existing attempts utilize adoptive transfer of γδ T cells as TILs, or as traditional CAR-T by putting CAR into γδ T cells, however, sophisticated γδ T engineering with strong potency and functionality is rare. The described GUAR is configured to recapitulate the native γδTCR signaling mechanism, but with a custom-designed antigen-binding domain that imparts a user-defined antigen recognition (i.e., with a universal antibody receptor to enable a potent and specific cell therapy).

Typically, the constant immunoglobulin domains of the gamma and delta TCR subunits are contiguous with a variable Ig (e.g., IgG) domain subunit at the amino (NH) terminus, and contiguous with, a transmembrane region at the carboxyl (COOH) terminus.

Any paired TCR gamma and delta constant domain subunits can be used in the GUAR molecules. In some embodiments, the TCR gamma constant domain (TCR Cγ) is fused to the Ig (e.g., IgG) variable heavy domain (VH) subunit, and the TCR delta constant domain (TCR Cδ) is fused to the Ig (e.g., IgG) variable light domain (V_{L} or Vₖ) subunit. In other embodiments, the TCR gamma constant domain (TCR Cγ) is fused to the Ig (e.g., IgG) variable light domain (V_{L} or Vₖ) subunit, and the TCR delta constant domain (TCR Cδ) is fused to the Ig (e.g., IgG) variable heavy domain (V_{H}) subunit.

An exemplary consensus amino acid sequence for the mature constant domain subunit of the TCR gamma polypeptide is:

(SEQ ID NO: 1)
DKQLDADVSPKPTIFLPSIAETKLQKAGTYLCLLEKFFPDV
IKIEWQEKKSNTILGSQEGNTMKTNDTYMKFSWLTVPEKSL
DKEHRCIVRHENNKNGVDQEIIFPPIKTDVITMDPKDNCSK
DANDTLLLQLTNTSAYYMYLLLLLKSVVYFAIITCCLLRRT
AFCCNGEKS.

The extracellular portion is believed to be amino acids 1-138, the transmembrane portion is believed to be amino acids 139-161 (bolded), and the intracellular domain is believed to be amino acids 162-173 (italics). See also UniProt accession number P0CF51⋅TRGC1_HUMAN.

In some forms, the mature constant domain subunit of the TCR gamma polypeptide is a variant having at least 75%, up to 99% identity to SEQ ID NO:1. For example in some forms, the variant sequence has at least about 75%, 80%, 85%, 90%, or 95% identity to SEQ ID NO:1. Therefore, in some forms, the variant TCR gamma polypeptide has an amino acid sequence that has one or more amino acids different to SEQ ID NO:1, such as one or more substitutions, deletions or additions at any one of the amino acid positions of SEQ ID NO:1.

An exemplary consensus amino acid sequence for the mature constant domain subunit of the TCR delta polypeptide is:

(SEQ ID NO: 2)
SQPHTKPSVFVMKNGTNVACLVKEFYPKDIRINLVSSKKI
TEFDPAIVISPSGKYNAVKLGKYEDSNSVTCSVQHDNKTV
HSTDFEVKTDSTDHVKPKETENTKQPSKSCHKPKAIVHTE
KVNMMSLTVLGLRMLFAKTVAVNELLTAKLEF L.

The extracellular portion is believed to be amino acids 1-129, the transmembrane portion is believed to be amino acids 130-152 (bolded), and the intracellular domain is believed to be amino acid 163 (italics). See also UniProt accession number B7Z8K6⋅TRDC_HUMAN.

In some forms, the mature constant domain subunit of the TCR delta polypeptide is a variant having at least 75%, up to 99% identity to SEQ ID NO:2. For example in some forms, the variant sequence has at least about 75%, 80%, 85%, 90%, or 95% identity to SEQ ID NO:2. Therefore, in some forms, the variant TCR delta polypeptide has an amino acid sequence that has one or more amino acids different to SEQ ID NO:2, such as one or more substitutions, deletions or additions at any one of the amino acid positions of SEQ ID NO:2.

### 2. Ig Fc-Specific Immunoglobulin Variable Domain

Typically, the variable heavy and light immunoglobulin domains (also referred to as antigen binding domains) from an antibody (e.g., Ig) developed to bind a target antibody isotype constant region, e.g., human IgG antibody constant region (Fc) bind to an epitope that is (i) highly conserved on the target isotype, e.g., human immunoglobulin IgG subtypes, and (ii) which does not, when bound by the GUAR, inhibit the antigen-binding activity of the antibody, e.g., IgG. Although the constant region of human IgG1 is a preferred target, other isotype and subtype constant regions, including but not limited to, IgG4, IgG2, IgG3, IgM, IgA, IgD, and IgE, are also expressly contemplated.

The term antibody herein refers to natural or synthetic polypeptides that bind a target antigen. The term includes polyclonal and monoclonal antibodies, including intact antibodies and functional (e.g., antigen-binding) antibody fragments, including Fab fragments, F(ab′)2 fragments, Fab′ fragments, Fv fragments, recombinant IgG (rlgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri scFv. The term also encompasses intact or full-length antibodies, including antibodies of any class or subclass, including IgG and sub classes thereof, IgM, IgE, IgA, and IgD. Thus, although typically discussed in the context of IgG, the target antibody of the Ig-Fc-specific immunoglobulin variable domain can be another IgG subtype, or isotype such as IgM, IgE, IgA, or IgD.

The antigen-binding domains typically contain complementary determining regions (CDR) of an antibody.

Typically, the variable immunoglobulin domain subunits are contiguous with a gamma or delta TCR constant domain subunit at the carboxyl (COOH) terminus.

Any paired immunoglobulin heavy and light domain subunits that selectively and specifically bind to the constant (Fc) region of a target antibody isotype, e.g., human IgG can be used in the GUAR molecules.

An exemplary consensus amino acid sequence for the mature variable kappa light subunit of the IgG Fc-binding immunoglobulin polypeptide is:

(SEQ ID NO: 3)
DIQMTQSPSSLSASVGDRVTITCRASQSISSYLNWYQQKPGKAPKLLIYVASSLQSGVP
SRFSGSGSGTNFTLTISSLQPEDFATYYCQQSYSTPITFGQGTRLEIKR.

In some forms, the mature variable kappa light subunit of the IgG Fc-binding immunoglobulin polypeptide is a variant having at least 75%, up to 99% identity to (SEQ ID NO:3). For example in some forms, the variant sequence has at least about 75%, 80%, 85%, 90%, or 95% identity to SEQ ID NO:3. Therefore, in some forms, the variable light subunit polypeptide has an amino acid sequence that has one or more amino acids different to SEQ ID NO:3, such as one or more substitutions, deletions or additions at any one of the amino acid positions of SEQ ID NO:3.

Typically, the variant is functional if it maintains the function of specific antigen binding. Therefore, the variant typically includes the three complementarity determining regions (CDRs L1-3) having an amino acid sequence of:

CDR-L1:
(SEQ ID NO: 27)
RASQSISSYLN;
CDR-L2:
(SEQ ID NO: 28)
VASSLQS;
and
CDR-L3:
(SEQ ID NO: 29)
QQSYSTPIT.

In some embodiments, there is little or no variation in the CDRs.

An exemplary consensus amino acid sequence for the mature variable heavy subunit of the IgG Fc-binding immunoglobulin polypeptide is:

(SEQ ID NO: 4)
QVQLQESGPGLVKPSETLSLTCTVSGDSISSYFWSWIRQPPGKGLEWIGYIYYSGTTNY
NPSLKSRLTISVDTSKNQFSLKLNSVTAADTAVYYCARDWGNSPFDYWGQGTLVTVSS.

In some forms, the mature variable heavy subunit of the IgG Fc-binding immunoglobulin polypeptide is a variant having at least 75%, up to 99% identity to (SEQ ID NO:4). For example in some forms, the variant sequence has at least about 75%, 80%, 85%, 90%, or 95% identity to SEQ ID NO:4. Therefore, in some forms, the variable heavy subunit polypeptide has an amino acid sequence that has one or more amino acids different to SEQ ID NO:4, such as one or more substitutions, deletions or additions at any one of the amino acid positions of SEQ ID NO:4.

Typically, the variant is functional if it maintains the function of specific antigen binding. Therefore, the variant typically includes the three complementarity determining regions (CDRs H1-3) having an amino acid sequence of:

CDR-H1:
(SEQ ID NO: 30)
SYFWS;
CDR-H2:
(SEQ ID NO: 31)
YIYYSGTTNYNPSLKS;
and
CDR-H3:
(SEQ ID NO: 32)
DWGNSPFDY.

In some embodiments, there is little or no variation in the CDRs.

Antibodies and antigen binding polypeptides thereof including the CDRs of SEQ ID NO:3 and/or SEQ ID NO:4 are also provided and can be used together or separately from other elements of the disclosed compositions. Thus, antibodies and antigen binding polypeptides including the CDRs of SEQ ID NO:3 (e.g., SEQ ID NOS:27, 28, and 29) containing within a light chain variable region framework and/or including the CDRs of SEQ ID NO:4 (e.g., SEQ ID NOS:30, 31, and 32) contained within a heavy chain variable region framework are provided. For example, the CDRs of SEQ ID NO:3 can be presented in a different light chain variable region framework, and/or the CDRs of SEQ ID NO:4 can be presented in a different heavy chain variable region framework, e.g., a humanized or chimeric framework. Typically the CDRs are presented in the same orientation as in SEQ ID NO:3 and/or SEQ ID NO:4 (e.g., CDR-L1 is SEQ ID NO:27, CDR-L2 is SEQ ID NO:28, and CDR-L3 is SEQ ID NO:29; and/or CDR-HT is SEQ ID NO:29, CDR-H2 is SEQ ID NO:30, and CDR-H3 is SEQ ID NO:31). The antibodies and antigen binding polypeptides can be full antibodies or fragments or synthetic fusion polypeptides that bind the target antigen. The antibodies and antigen binding polypeptides can be humanized or chimeric. Exemplary fragments and fusions include, but are not limited to, single chain antibodies, single chain variable fragments (scFv), disulfide-linked Fvs (sdFv), Fab′, F(ab′)2, Fv, and single domain antibody fragments (sdAb). In some embodiments, the molecule includes two or more scFv. The disclosed anti-IgG antibodies and antigen binding polypeptides can also be further modified as discussed in more detail below with respect anti-GPRC5D antibodies.

### 3. Masking Moieties

In some forms, the GUAR molecule includes one or more additional moieties that physically occludes or “masks” the IgG Fc-binding function of the GUAR, i.e., a “masking” moiety. Typically, the masking moiety is reversibly associated with the GUAR molecule for example, such that the masking moiety can be removed and the IgG Fc-binding functionality of the GUAR restored.

Exemplary masking moieties include polypeptides, carbohydrates, nucleic acids. Lipids and small molecules. In a preferred form, the masking moiety is a coiled-coil peptide. Exemplary coiled-coil peptide masking moieties include CC2B, CC3, CC4, and CC5. Typically, the masking peptide is fused to the amino (NH) terminus of the human IgG Fc-specific immunoglobulin variable domain.

In some forms, the making moiety is associated with the GUAR via a linker, such as a cleavable linker. An exemplary cleavable linker is a protease-cleavable linker. In some forms, the protease cleavable linker is receptive to enzymatic cleavage by a protease that is located or restricted to one or more tissues, organs or specific tissue types. An exemplary tissue-restricted protease is a tumor-associated protease, such as that which is present in the tumor microenvironment (TME). In some forms, the protease-cleavable moiety is cleavable by urokinase plasminogen activator (uPA, a serine protease (SP)). An exemplary uPA-sensitive amino acid sequence is LSGRSDNH (SEQ ID NO:5)

### 4. Co-Stimulatory Intracellular Domain

In some forms, the costimulatory domain or intracellular domain from the cytokine receptor is reconstituted to the C terminal of the GUAR molecule. For example, in some forms, the GUAR includes one or more intracellular domains from a co-stimulatory factor or other molecule attached to the carboxyl terminus of the TCR Cγ subunit, or the carboxyl terminus of the TCR Cδ subunit, or both. In some forms, the GUAR includes intracellular domains from a co-stimulatory factor selected from CD28, OX40, ICOS, IL7Rα, or ΔIL2Rβ fused to the carboxyl terminus of the TCR δ subunit, or to the carboxyl terminus of the TCR γ subunit, or to both the TCR δ subunit and TCR γ subunit. In an exemplary form, the CD28 intracellular domain is fused to the intracellular component of the TCR delta constant domain, and the intracellular domain of a co-stimulatory factor selected from OX40, ICOS, IL7Rα, or ΔIL2Rβ is fused to the carboxyl terminus of the TCR gamma subunit.

The intracellular signaling domain is responsible for activation of at least one of the normal effector functions of the immune effector cell expressing the GUAR. The term effector function refers to a specialized function of a cell. Effector function of a T cell, for example, may be cytolytic activity or helper activity including the secretion of cytokines. In some forms, an intracellular signaling domain includes the zeta chain of the T cell receptor or any of its homologs (e.g., eta, delta, gamma or epsilon), MBl chain, B29, Fc RIII, Fc RI and combinations of signaling molecules such as CD3ζ and CD28, 4 1BB, OX40 and combination thereof, as well as other similar molecules and fragments. Intracellular signaling portions of other members of the families of activating proteins can be used, such as FcγRIII and FcεRI.

Many immune effector cells require co-stimulation, in addition to stimulation of an antigen-specific signal, to promote cell proliferation, differentiation and survival, as well as to activate effector functions of the cell. Therefore, in some forms, the GUAR includes at least one co-stimulatory signaling domain. The term co-stimulatory signaling domain, refers to at least a portion of a protein that mediates signal transduction within a cell to induce an immune response such as an effector function. The co-stimulatory signaling domain can be a cytoplasmic signaling domain from a co-stimulatory protein, which transduces a signal and modulates responses mediated by immune cells, such as T cells, NK cells, macrophages, neutrophils, or eosinophils. In some forms, the co-stimulatory signaling domain is derived from a co-stimulatory molecule selected from CD27, CD28, CD137, 0X40, CD30, CD40, CD3, LFA 1, ICOS, CD2, CD7, LIGHT, NKG2C, B7-H3, ligands of CD83 and combinations thereof.

Exemplary consensus amino acid sequence for various mature intracellular domain polypeptides and their encoding nucleic acids are provided below as SEQ ID NOS:83-110. Such sequences and functional fragments thereof having, for example, at least 70, 75, 80, 85, 90, or 95 percent sequence identity thereto are provided and can be used as/form part of the disclosed GUAR constructs as provided herein.

CD27 ICD
DNA-CDS
(SEQ ID NO: 97)
CAACGAAGGAAATATAGATCAAACAAAGGAGAAAGTCCTGTGGAGCCTGCAGAGCCTTG
TCATTACAGCTGCCCCAGGGAGGAGGAGGGCAGCACCATCCCCATCCAGGAGGATTACC
GAAAACCGGAGCCTGCCTGCTCCCCC
Amino acid
(SEQ ID NO: 83)
QRRKYRSNKGESPVEPAEPCHYSCPREEEGSTIPIQEDYRKPEPACSP
CD28 ICD
DNA-CDS
(SEQ ID NO: 98)
AGGAGTAAGAGGAGCAGGCTCCTGCACAGTGACTACATGAACATGACTCCCCGCCGCCC
CGGGCCCACCCGCAAGCATTACCAGCCCTATGCCCCACCACGCGACTTCGCAGCCTATC
GCTCC
Amino acid
(SEQ ID NO: 84)
RSKRSRLLHSDYMNMTPRRPGPTRKHYQPYAPPRDFAAYRS
CD137 ICD
DNA-CDS
(SEQ ID NO: 99)
AAACGGGGCAGAAAGAAACTCCTGTATATATTCAAACAACCATTTATGAGACCAGTACA
AACTACTCAAGAGGAAGATGGCTGTAGCTGCCGATTTCCAGAAGAAGAAGAAGGAGGAT
GTGAACTG
Amino acid
(SEQ ID NO: 85)
KRGRKKLLYIFKQPFMRPVQTTQEEDGCSCRFPEEEEGGCEL
OX40 ICD
DNA-CDS
(SEQ ID NO: 100)
GCCCTGTACCTGCTCCGGAGGGACCAGAGGCTGCCCCCCGATGCCCACAAGCCCCCTGG
GGGAGGCAGTTTCCGGACCCCCATCCAAGAGGAGCAGGCCGACGCCCACTCCACCCTGG
CCAAGATC
Amino acid
(SEQ ID NO: 86)
ALYLLRRDQRLPPDAHKPPGGGSFRTPIQEEQADAHSTLAKI
CD30 ICD
DNA-CDS
(SEQ ID NO: 101)
TGCCACCGGAGGGCCTGCAGGAAGCGAATTCGGCAGAAGCTCCACCTGTGCTACCCGGT
CCAGACCTCCCAGCCCAAGCTAGAGCTTGTGGATTCCAGACCCAGGAGGAGCTCAACGC
AGCTGAGGAGTGGTGCGTCGGTGACAGAACCCGTCGCGGAAGAGCGAGGGTTAATGAGC
CAGCCACTGATGGAGACCTGCCACAGCGTGGGGGCAGCCTACCTGGAGAGCCTGCCGCT
GCAGGATGCCAGCCCGGCCGGGGGCCCCTCGTCCCCCAGGGACCTTCCTGAGCCCCGGG
TGTCCACGGAGCACACCAATAACAAGATTGAGAAAATCTACATCATGAAGGCTGACACC
GTGATCGTGGGGACCGTGAAGGCTGAGCTGCCGGAGGGCCGGGGCCTGGCGGGGCCAGC
AGAGCCCGAGTTGGAGGAGGAGCTGGAGGCGGACCATACCCCCCACTACCCCGAGCAGG
AGACAGAACCGCCTCTGGGCAGCTGCAGCGATGTCATGCTCTCAGTGGAAGAGGAAGGG
AAAGAAGACCCCTTGCCCACAGCTGCCTCTGGAAAG
Amino acid
(SEQ ID NO: 87)
CHRRACRKRIRQKLHLCYPVQTSQPKLELVDSRPRRSSTQLRSGASVTEPVAEERGLMS
QPLMETCHSVGAAYLESLPLQDASPAGGPSSPRDLPEPRVSTEHTNNKIEKIYIMKADT
VIVGTVKAELPEGRGLAGPAEPELEEELEADHTPHYPEQETEPPLGSCSDVMLSVEEEG
KEDPLPTAASGK
CD40 ICD
DNA-CDS
(SEQ ID NO: 102)
AAAAAGGTGGCCAAGAAGCCAACCAATAAGGCCCCCCACCCCAAGCAGGAACCCCAGGA
GATCAATTTTCCCGACGATCTTCCTGGCTCCAACACTGCTGCTCCAGTGCAGGAGACTT
TACATGGATGCCAACCGGTCACCCAGGAGGATGGCAAAGAGAGTCGCATCTCAGTGCAG
GAGAGACAG
Amino acid
(SEQ ID NO: 88)
KKVAKKPTNKAPHPKQEPQEINFPDDLPGSNTAAPVQETLHGCQPVTQEDGKESRISVQ
ERQ
LFA-1 ICD
DNA-CDS
(SEQ ID NO: 103)
AAGGCTCTGATCCACCTGAGCGACCTCCGGGAGTACAGGCGCTTTGAGAAGGAGAAGCT
CAAGTCCCAGTGGAACAATGATAATCCCCTTTTCAAGAGCGCCACCACGACGGTCATGA
ACCCCAAGTTTGCTGAGAGT
Amino acid
(SEQ ID NO: 89)
KALIELSDLREYRRFEKEKLKSQWNNDNPLFKSATTTVMNPKFAES
ICOS ICD
DNA-CDS
(SEQ ID NO: 104)
TGTTGGCTTACAAAAAAGAAGTATTCATCCAGTGTGCACGACCCTAACGGTGAATACAT
GTTCATGAGAGCAGTGAACACAGCCAAAAAATCTAGACTCACAGATGTGACCCTA
Amino acid
(SEQ ID NO: 90)
CWLTKKKYSSSVHDPNGEYMFMRAVNTAKKSRLTDVTL
CD2 ICD
DNA-CDS
(SEQ ID NO: 105)
AAAAGGAAAAAACAGAGGAGTCGGAGAAATGATGAGGAGCTGGAGACAAGAGCCCACAG
AGTAGCTACTGAAGAAAGGGGCCGGAAGCCCCACCAAATTCCAGCTTCAACCCCTCAGA
ATCCAGCAACTTCCCAACATCCTCCTCCACCACCTGGTCATCGTTCCCAGGCACCTAGT
CATCGTCCCCCGCCTCCTGGACACCGTGTTCAGCACCAGCCTCAGAAGAGGCCTCCTGC
TCCGTCGGGCACACAAGTTCACCAGCAGAAAGGCCCGCCCCTCCCCAGACCTCGAGTTC
AGCCAAAACCTCCCCATGGGGCAGCAGAAAACTCATTGTCCCCTTCCTCTAAT
Amino acid
(SEQ ID NO: 91)
KRKKQRSRRNDEELETRAHRVATEERGRKPHQIPASTPQNPATSQHPPPPPGHRSQAPS
HRPPPPGHRVQHQPQKRPPAPSGTQVHQQKGPPLPRPRVQPKPPEGAAENSLSPSSN
CD7 ICD
DNA-CDS
(SEQ ID NO: 106)
AGGACACAGATAAAGAAACTGTGCTCGTGGCGGGATAAGAATTCGGCGGCATGTGTGGT
GTACGAGGACATGTCGCACAGCCGCTGCAACACGCTGTCCTCCCCCAACCAGTACCAG
Amino acid
(SEQ ID NO: 92)
RTQIKKLCSWRDKNSAACVVYEDMSHSRCNTLSSPNQYQ
LIGHT ICD
DNA-CDS
(SEQ ID NO: 107)
ATGGAGGAGAGTGTCGTACGGCCCTCAGTGTTTGTGGTGGATGGACAGACCGACATCCC
ATTCACGAGGCTGGGACGAAGCCACCGGAGACAGTCGTGCAGTGTGGCCCGG
Amino acid
(SEQ ID NO: 93)
MEESVVRPSVFVVDGQTDIPFTRLGRSHRRQSCSVAR
NKG2C-ICD
DNA-CDS
(SEQ ID NO: 108)
ATGAATAAACAAAGAGGAACCTTCTCAGAAGTGAGTCTGGCCCAGGACCCAAAGCGGCA
GCAAAGGAAACCTAAAGGCAATAAAAGCTCCATTTCAGGAACCGAACAGGAAATATTCC
AAGTAGAATTAAATCTTCAAAATCCTTCCCTGAATCATCAAGGGATTGATAAAATATAT
GACTGCCAAGGTTTACTGCCACCTCCAGAGAAG
Amino acid
(SEQ ID NO: 94)
MNKQRGTFSEVSLAQDPKRQQRKPKGNKSSISGTEQEIFQVELNLQNPSLNHQGIDKIY
DCQGLLPPPEK
B7H3 ICD
DNA-CDS
(SEQ ID NO: 109)
TGCTGGAGAAAGATCAAACAGAGCTGTGAGGAGGAGAATGCAGGAGCTGAGGACCAGGA
TGGGGAGGGAGAAGGCTCCAAGACAGCCCTGCAGCCTCTGAAACACTCTGACAGCAAAG
AAGATGATGGACAAGAAATAGCC
Amino acid
(SEQ ID NO: 95)
CWRKIKQSCEEENAGAEDQDGEGEGSKTALQPLKHSDSKEDDGQEIA
CD3z ICD
DNA-CDS
(SEQ ID NO: 110)
AGAGTGAAGTTCAGCAGGAGCGCAGACGCCCCCGCGTACCAGCAGGGCCAGAACCAGCT
CTATAACGAGCTCAATCTAGGACGAAGAGAGGAGTACGATGTTTTGGACAAGAGACGTG
GCCGGGACCCTGAGATGGGGGGAAAGCCGCAGAGAAGGAAGAACCCTCAGGAAGGCCTG
TACAATGAACTGCAGAAAGATAAGATGGCGGAGGCCTACAGTGAGATTGGGATGAAAGG
CGAGCGCCGGAGGGGCAAGGGGCACGATGGCCTTTACCAGGGTCTCAGTACAGCCACCA
AGGACACCTACGACGCCCTTCACATGCAGGCCCTGCCCCCTCGC
Amino acid
(SEQ ID NO: 96)
RVKFSRSADAPAYQQGQNQLYNELNLGRREEYDVLDKRRGRDPEMGGKPQRRKNPQEGL
YNELQKDKMAEAYSEIGMKGERRRGKGHDGLYQGLSTATKDTYDALHMQALPPR

### 5. Exemplary GUAR Polypeptides

GUAR-Arm-1 (GUAR-VK human TCR delta chain constant)
(SEQ ID NO: 6)

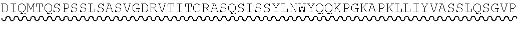

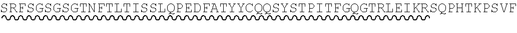

VMKNGTNVACLVKEFYPKDIRINLVSSKKITEFDPAIVISPSGKYNAVKLGKYEDSNSV
TCSVQHDNKTVHSTDFEVKTDSTDHVKPKETENTKQPSKSCHKPKAIVHTEKVNMMSLT
VLGLRMLFAKTVAVNFLLTAKLFFL

The extracellular domain includes amino acids at positions 1-237 with the IgG1 Fc-binding domain underlined; the transmembrane domain includes amino acids at positions 238-260, and the cytoplasmic domain includes amino acids at positions 261.

GUAR-Arm-2 (GUAR-VH-human TCR gamma chain constant)
(SEQ ID NO: 7)

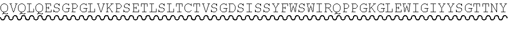

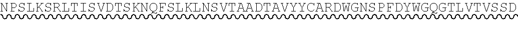

KQLDADVSPKPTIFLPSIAETKLQKAGTYLCLLEKFFPDVIKIHWQEKKSNTILGSQED
NTMKTNDTYMKFSWLTVPEKSLDKEHRCIVRHENNKNGVDQEIIFPPIKTDVITMDPKD
NCSKDANDTLLLQLTNTSAYYMYLLLLLKSVVYFAIITCCLLRRTAFCCNGEKS

The extracellular domain includes amino acids at positions 1-255 (Fc-binding domain in underlining); the transmembrane domain includes amino acids at positions 256-278, and the cytoplasmic domain includes amino acids at positions 279-290.

### 6. Exemplary GUAR Constructs and Proteins

Exemplary nucleic acid constructs encoding GUAR molecules and components thereof are provided. In some forms, a nucleic acid construct expresses a polypeptide encoding the TCR gamma or TCR delta subunit of a GUAR, or both the TCR gamma and TCR delta subunits of a GUAR. In some forms, a construct includes, from 5′ to 3′, a nucleic acid encoding the variable light (e.g., kappa) domain subunit of a human IgG Fc-specific IgG fused to the human TCR delta constant domain, fused to the CD28 intracellular domain. In other forms, a construct includes, from 5′ to 3′, a nucleic acid encoding the variable heavy domain subunit of a human IgG Fc-specific IgG fused to the human TCR gamma constant domain, fused to the intracellular domain of a co-stimulatory factor selected from OX40, ICOS, IL7Rα, or ΔIL2Rβ. In some forms, the GUAR molecules are designed as a transposon for incorporation within the genome of a suitable host cell, such as a T cell. Typically, a transposon designed for insertion and expression within a host T cell includes, from 5′ to 3′, a nucleic acid encoding the variable light (e.g., kappa) domain subunit of a human IgG Fc-specific IgG fused to the human TCR delta constant domain, fused to the CD28 intracellular domain, then a self-cleaving peptide sequence, e.g., P2A, E2A, Furin 2A (F2A), or T2A and the variable heavy domain subunit of a human IgG Fc-specific IgG fused to the human TCR gamma constant domain, fused to the intracellular domain of a co-stimulatory factor selected from OX40, ICOS, IL7Rα, or ΔIL2Rβ.

a. GUAR Domain Variants

It has been established that the GUAR polypeptide is sufficient to drive enhanced gamma-delta T cell mediated killing of an IgG-targeted cell in vivo. Thus, compositions and methods of use of GUAR peptides and functional variants thereof are provided. Functional variants of GUAR can be, for example, variants incorporating any number of amino acids substitutions, additions and/or deletions that sustain and/or improve the observed function of GUAR.

In some forms, the functional variant of GUAR includes a minimal sequence identity to any one of SEQ ID NOs:1-2, optionally light and heavy chain variable regions including the CDRs of SEQ ID NOS:3 and 4, or SEQ ID NOS:3 and/or 4. For example, the functional variant of GUAR can include up to 50%, 40%, 40%, 20% 10%, 5%, 4%, 3%, 2%, 1% or less than 1% variation in the amino acid sequence of any one or more of the TCR Cγ domain, the IgG variable heavy domain (VH) subunit, the TCR Cδ domain, or the IgG variable light domain (VL) subunit. Exemplary variant GUAR molecules include changes to a total number of amino acids including, but not limited to 20, 25, 30, 35, 36, 40, 41, 45, 50, 60, 66, 70, 75, 100, 125, 150, or 175 amino acids. In some forms, variants of GUAR have a functional activity that is increased or reduced by a certain amount relative to the GUAR that is set forth by SEQ ID NOs:1-4. For example, in some forms, a GUAR variant has a functional activity that is at least 65%, 70%, 75%, 80%, 85%, 90%, 95%, 99% or 100% of the GUAR that is set forth by SEQ ID NOs 1-4. Variants can have, for example, at least 65%, 70%, 75%, 80%, 85%, 90%, 95%, 99% or 100% sequence identity to any one of SEQ ID NOs:1-10, or a functional fragment thereof. In a particular form, a variant has a TCR Cγ domain having at least 75%, 80%, 85%, 90%, 95%, 99% or 100% sequence identity to SEQ ID NO:1. In a particular form, a variant has a TCR Cδ domain having at least 75%, 80%, 85%, 90%, 95%, 99% or 100% sequence identity to SEQ ID NO:2. In a particular form, a variant has a VH domain having at least 75%, 80%, 85%, 90%, 95%, 99% or 100% sequence identity to SEQ ID NO:3. In a particular form, a variant has a VL domain having at least 75%, 80%, 85%, 90%, 95%, 99% or 100% sequence identity to SEQ ID NO:4.

Preferably variants maintain the ability to interact with the target isotype constant domain, e.g., IgG Fc, i.e., maintain the specific epitope in the variable regions of the GUAR. In some forms, a GUAR variant is considered to be “functional” if it maintains the ability to interact with the Ig constant domain, e.g., IgG-Fc, and maintains the ability to activate one or more immune functions of the T cell in response to binding. In some forms, GUAR and variants thereof are identified as functional if they interact with IgG-Fc and initiate degranulation of the GUAR-T cell.

Typically, amino acid substitutions within GUAR peptides are based on the relative similarity of the amino acid side-chain substituents, for example, their hydrophobicity, hydrophilicity, charge, size, and the like. Exemplary substitutions of amino acids within any of SEQ ID NOS: 1-4 can include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Forms of this disclosure thus contemplate functional or biological equivalents of a GUAR polypeptide, as set forth in any one of SEQ ID NOS:1-4. In particular forms, the GUAR polypeptides can include variants having about 50%, 60%, 70%, 80%, 90%, and 95% sequence identity to the polypeptide of any one of SEQ ID NOS:1-4.

### b. 2A Peptides

In some forms, constructs encoding GUAR molecules include one or more polypeptide sequences encoding a viral 2A region. Therefore, in some forms, constructs encoding GUAR molecules include one or more 2A peptide sequences, typically at the carboxyl (C) terminus of the TCR delta constant domain.

T2A peptides are 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (Thosea asigna virus 2A) were also identified. The mechanism of 2A-mediated “self-cleavage” was recently discovered to be ribosome skipping the formation of a glycyl-prolyl peptide bond at the C-terminus of the 2A20). A highly conserved sequence GDVEXNPGP (SEQ ID NO:8) is shared by different 2As at the C-terminus, and is important for the creation of steric hindrance and ribosome skipping. There are three possibilities for a 2A-mediated skipping event:
1—Successful skipping and recommencement of translation results in two “cleaved” proteins: the protein upstream of the 2A is attached to the complete 2A peptide except for the C-terminal proline, and the protein downstream of the 2A is attached to one proline at the N-terminus;
2—Successful skipping but ribosome fall-off and discontinued translation results in only the protein upstream of 2A; and
3—Unsuccessful skipping and continued translation resulting in a fusion protein.

Overall, 2A peptides lead to relatively high levels of downstream protein expression compared to other strategies for multi-gene co-expression, and they are small in size thus bearing a lower risk of interfering with the function of co-expressed genes. 2A peptides have also been successfully employed by several different groups for polycistronic and bi-cistronic multigene expression.

Therefore, in some forms, GUAR domains for co-expression as multi-subunit proteins are coupled to one or more 2A polypeptide sequences.

An exemplary amino acid sequence for a T2A sequence is:

(SEQ ID NO: 11)
GSGSGEGRGSLLTCGDVEENPGP.

c. Additional Subunits, Etc.

Heterologous elements that can be associated with, linked, conjugated, or otherwise attached directly or indirectly to the GUAR sequence(s), or nucleic acids expressing the GUAR polypeptides are disclosed. Such molecules include, but are not limited to, protein domains, such as transduction domains, fusogenic peptides, targeting molecules, and sequences that enhance protein expression and/or isolation.

In some forms, GUAR peptides include one or more heterologous peptide domains, such as receptors at the surface of a cell, optionally including a transmembrane domain that anchors or connects the ectodomain to the cell surface and connects with the one or more intracellular GUAR. Exemplary cell surface receptors coordinate the activity of cells upon interaction with other cells, such as immune cells, such as T cells. For example, in some forms, the heterologous domain is a recombinant or engineered Programmed death protein 1 (PD1) domain. In other forms, the heterologous domain is a T2A sequence that enhances cell expression, and one or more leader sequences, such as a CD8 leader sequence.

In some forms, the intracellular signaling domains mediating GUAR-T cell activation can include a CD3ζ co-receptor signaling domain derived from the C-region of the TCR gamma and delta subunits and one or more costimulatory domains.

In some forms, the GUAR includes one or more spacer domain(s) (also referred to as hinge domain) that is located between the extracellular domain and the transmembrane domain. A spacer domain is an amino acid segment that is generally found between two domains of a protein and may allow for flexibility of the protein and movement of one or both of the domains relative to one another. Any amino acid sequence that provides such flexibility and movement of the extracellular domain relative to the transmembrane domain can be used. The spacer domain can be a spacer or hinge domain of a naturally occurring protein. In some forms, the hinge domain is derived from CD8a, such as, a portion of the hinge domain of CD8a, e.g., a fragment containing at least 5 (e.g., 5, 10, 15, 20, 25, 30, 35, or 40) consecutive amino acids of the hinge domain of CD8a. Hinge domains of antibodies, such as an IgG, IgA, IgM, IgE, or IgD antibodies can also be used. In some forms, the hinge domain is the hinge domain that joins the constant CH1 and CH2 domains of an antibody. Non-naturally occurring peptides may also be used as spacer domains. For example, the spacer domain can be a peptide linker, such as a (GxS)n linker, wherein x and n, independently can be an integer of 3 or more, including 3, 4, 5, 6, 7, 8, 9, 10, 11, 12, or more. The term “linker” as used herein includes, without limitation, peptide linkers. The peptide linker can be any size provided it does not interfere with the binding of the epitope by the variable regions. In some forms, the linker includes one or more glycine and/or serine amino acid residues. In some forms, the linker includes a glycine-glutamic acid di-amino acid sequence. For example, a linker can include 4-8 amino acids. In a particular form, a linker includes the amino acid sequence GQSSRSS (SEQ ID NO:12). In another form, a linker includes 15-20 amino acids, for example 18 amino acids. Other flexible linkers include, but are not limited to, the amino acid sequences Gly-Ser, Gly-Ser-Gly-Ser (SEQ ID NO:13), Ala-Ser, Gly-Gly-Gly-Ser (SEQ ID NO:81), (Gly4-Ser)2 (SEQ ID NO:14) and (Gly4-Ser)4 (SEQ ID NO:15), and (Gly-Gly-Gly-Gly-Ser)3 (SEQ ID NO:16).

In some forms, the GUAR includes a transmembrane domain that can be directly or indirectly fused to the antigen-binding domain. The transmembrane domain may be derived either from a natural or a synthetic source. In some forms, the transmembrane domain of the GUAR is modified to include a transmembrane domain of CD8, CD4, CD28, CD137, CD80, CD86, CD152 (CTLA-4) or PD1, or a portion thereof.

Transmembrane domains can also contain at least a portion of a synthetic, non-naturally occurring protein segment. In some forms, the transmembrane domain is a synthetic, non-naturally occurring alpha helix or beta sheet. In some forms, the protein segment is at least about 15 amino acids, e.g., at least 15, 16, 17, 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776.

In some forms, the GUAR molecules include one or more additional elements that are designed to assist and/or enhance the expression or production of the GUAR. Although many proteins with therapeutic or commercial uses can be produced by recombinant organisms, the yield and quality of the expressed protein are variable due to many factors. For example, heterologous protein expression by genetically engineered organisms can be affected by the size and source of the protein to be expressed, the presence of an affinity tag linked to the protein to be expressed, codon biasing, the strain of the microorganism, the culture conditions of microorganism, and the in vivo degradation of the expressed protein. Some of these problems can be mitigated by fusing the protein of interest to an expression or solubility enhancing amino acid sequence. Exemplary expression or solubility enhancing amino acid sequences include maltose-binding protein (MBP), glutathione S-transferase (GST), thioredoxin (TRX), NUS A, ubiquitin (Ub), and a small ubiquitin-related modifier (SUMO).

In some forms, the compositions disclosed herein include expression or solubility enhancing amino acid sequence. In some forms, the expression or solubility enhancing amino acid sequence is cleaved prior administration of the composition to a subject in need thereof. The expression or solubility enhancing amino acid sequence can be cleaved in the recombinant expression system, or after the expressed protein in purified.

### 7. Nucleic Acids

Nucleic acids and vectors encoding or expressing GUAR proteins are also described.

a. Isolated Nucleic Acid Molecules Encoding GUAR

Isolated nucleic acids encoding GUAR polypeptides are disclosed. The term “isolated nucleic acid” refers to a nucleic acid that is separated from other nucleic acid molecules that are present in a mammalian genome, including nucleic acids that normally flank one or both sides of the nucleic acid in a mammalian genome. An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence encoding a GUAR polypeptide subunit. Thus, nucleic acids encoding each of the disclosed polypeptide sequences, and fragments and variants thereof, in sense and antisense, and in single stranded and double stranded forms, are provided. The nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

b. Vectors Expressing or Encoding GUAR

In some forms, nucleic acids encoding GUAR molecules are present within vectors. Vectors including an isolated polynucleotide encoding a GUAR polypeptide for the expression of a GUAR within a host cell are described.

The term “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). A vector can be a DNA vector or an RNA vector. In some forms, a vector is a DNA plasmid. One of ordinary skill in the art can construct a vector of the application through standard recombinant techniques in view of the present disclosure.

In some forms, the vector including nucleic acids encoding a GUAR is an expression vector. The term “expression vector” refers to any type of genetic construct including a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

In some forms, vectors contain one or more regulatory sequences. The term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability).

In some forms, the vector is a non-viral vector. Examples of non-viral vectors include, but are not limited to, DNA plasmids, bacterial artificial chromosomes, yeast artificial chromosomes, bacteriophages, etc. Examples of non-viral vectors include, but are not limited to, RNA replicon, mRNA replicon, modified mRNA replicon or self-amplifying mRNA, closed linear deoxyribonucleic acid, e.g., a linear covalently closed DNA, e.g., a linear covalently closed double stranded DNA molecule. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically include an origin of replication, a multiple cloning site, and a selectable marker, which for example, can be an antibiotic resistance gene. Examples of suitable DNA plasmids that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC®. complete baculovirus expression system (Thermo Fisher Scientific), which can be used for production and/or expression in insect cells; pcDNA™. or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pcDNA™, pcDNA3™, pVAX, pVAX-1, ADVAX, NTC8454, etc. In some forms, an expression vector is based on pVAX-1, which can be further modified to optimize protein expression in mammalian cells. pVAX-1 is a commonly used plasmid in DNA vaccines, and contains a strong human immediate early cytomegalovirus (CMV-IE) promoter followed by the bovine growth hormone (bGH)-derived polyadenylation sequence (pA). pVAX-1 further contains a pUC origin of replication and a kanamycin resistance gene driven by a small prokaryotic promoter that allows for bacterial plasmid propagation.

In some forms the vector is a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, arenavirus viral vectors, replication-deficient arenavirus viral vectors or replication-competent arenavirus viral vectors, bi-segmented or tri-segmented arenavirus, infectious arenavirus viral vectors, nucleic acids which include an arenavirus genomic segment wherein one open reading frame of the genomic segment is deleted or functionally inactivated (and replaced by a nucleic acid encoding a GUAR polypeptide or another therapeutic polypeptide as described herein), arenavirus such as lymphocytic choriomeningitidis virus (LCMV), e.g., clone 13 strain or MP strain, and arenavirus such as Junin virus e.g., Candid #1 strain, etc.

In some forms, the viral vector is an adenovirus vector, e.g., a recombinant adenovirus vector. A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other forms, an adenovirus vector is a rhAd vector, e.g. rhAd51, rhAd52 or rhAd53. In some forms, a recombinant viral vector is prepared using methods known in the art in view of the present disclosure. For example, in view of the degeneracy of the genetic code, several nucleic acid sequences can be designed that encode the same polypeptide. In some forms, a polynucleotide encoding a GUAR polypeptide is codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

In some forms, the vectors, e.g., a DNA plasmid or a viral vector (particularly an adenoviral vector), include any regulatory elements to establish conventional function(s) of the vector, including but not limited to replication and expression of the GUAR polypeptide encoded by the polynucleotide sequence of the vector.

In some forms, the vector is adeno-associated viral vector (AAV). AAV vector used in the compositions and methods can be a naturally occurring serotype of AAV or an artificial variant. In preferred forms, the serotype of the AAV vector is AAV6 or AAV9.

c. Transposons

In some forms, the vector is an AAV vector that can transduce diverse cell types with minimal cellular toxicity, leading to highly efficient and stable genomic modifications.

An exemplary method for introducing a GUAR into a cell includes introducing to the cell a viral vector including a transposon encoding the GUAR and a sequence that encodes one or more transposase enzymes configured to specifically mediate targeted integration of the transposon into the cellular genome.

Also disclosed are systems for introducing a GUAR into a cell, where the system includes a viral vector including a transposon encoding the GUAR and a sequence that encodes one or more transposase enzymes configured to specifically mediate targeted integration of the transposon into the cellular genome.

d. Multiplexed Genome Engineering Systems

In some forms, the expression vector also includes one or more additional functional elements, for example, for genetic modification of the host cell by removal or silencing of one or more of the host genes.

In some forms, the vector provides combinations of simultaneous multiplexed knockout and knock-in genomic modifications in the host cell. In some forms, the compositions include an RNA-guided endonuclease and one or more AAV vectors containing a sequence (e.g., a crRNA array) that encodes one or more crRNAs that collectively direct the endonuclease to one or more target genes. Optionally, at least one of the AAV vectors contains or further contains one or more HDR templates. The crRNA array can encode two or more crRNAs each of which direct the endonuclease to a different target gene. In some forms, the method can involve introducing two AAV vectors. In the foregoing method, the one or more HDR templates include (a) a sequence that encodes a reporter gene and/or a GUAR, and (b) one or more sequences homologous to one or more target sites. The HDR template can further include a promoter and/or polyadenylation signal operationally linked to each reporter gene, GUAR, or combination thereof.

In some forms, the RNA-guided endonuclease is capable of disruption of the target genes and/or the one or more HDR templates can mediate targeted integration of the reporter gene, the GUAR, or combinations thereof at the target sites. A target site can be within the locus of the disrupted gene or at a locus different from the disrupted gene. Exemplary target genes or target sites include, but are not limited to PDCD1, TRAC, CTLA4, B2M, CIITA, TRBC1, and TRBC2. A preferred target gene is TRAC. In some forms, the PDCD1 and/or TRAC gene can be disrupted; one or more reporter genes, and/or GUAR, can be integrated in the PDCD1 and/or TRAC gene; the PDCD1 gene can be disrupted and the one or more reporter genes, and/or GUAR can be integrated in the TRAC gene; or the TRAC gene can disrupted and the one or more reporter genes, and/or GUAR can be integrated in the PDCD1 gene. In some forms, the VL-Cδ-F2A-VH-Cγ sequences are amplified from lentiviral transgene plasmids and subcloned into AAV expression vectors allowing VL-Cδ and VH-Cγ expression under the control of TCRα promoter.

### 8. Exemplary GUAR Constructs

Exemplary constructs for the expression of GUAR at the surface of mammalian cells are provided. As described in the examples, several functional GUAR constructs have been expressed at the surface of human T-cells, including Jurkat cells.

An exemplary GUAR construct includes the nucleic acid sequence:

(SEQ ID NO: 33)
atggccttaccagtgaccgccttgctcctgccgctggccttgctgctccacgccgccag
gccgGAACAGAAACTGATTTCCGAGGAAGATCTGGATATCCAGATGACACAGACTACAT
CCTCCCTGTCTGCCTCTCTGGGAGACAGAGTCACCATCAGTTGCAGGGCAAGTCAGGAC
ATTAGCAATTATTTAAACTGGTATCAGCAGAAACCAGATGGAACTGTTAAACTCCTGAT
CTACTACACATCAAGATTACACTCAGGAGTCCCATCAAGGTTCAGTGGCAGTGGGTCTG
GAACAGATTATTCTCTCACCATTAGCAACCTGGAGCAAGAAGATATTGCCACTTACTTT
TGCCAACAGGGTAATACGCTTCCGTACACGTTCGGAGGGGGGACCAAGCTGGAAATAAA
GCGTagccagccccacaccaagcccagcgtgttcgtgatgaagaacggcaccaacgtgg
cctgcctggtgaaagagttctaccccaaggacatccggatcaacctggtgtccagcaag
aagatcaccgagttcgaccccgccatcgtgatcagccccagcggcaagtacaacgccgt
gaagctgggcaagtacgaggacagcaacagcgtgacctgcagcgtgcagcacgacaaca
agaccgtgcacagcaccgacttcgaggtgaaaaccgactccaccgaccacgtgaagccc
aaagagaccgagaacaccaagcagcccagcaagagctgccacaagcccaaggccatcgt
gcacaccgagaaggtgaacatgatgagcctgaccgtgctgggcctgcggatgctgttcg
ccaagacagtggccgtgaacttcctgctgaccgccaagctgttcttcctgCGACGGAAG
AGATCGGGTTCCGGCgccccggtgaaacagactttgaattttgaccttctcaagttggc
gggagacgtggagtccaacccagggccgATGTGGCTTCAGTCACTCCTTCTCTTGGGTA
CTGTTGCTTGCAGCATCTCCGATTACAAAGACGATGACGATAAGGAGGTGAAGCTGATG
GAGTCTGGGGGAGGCTTAGTGAAGCCTGGAGGGTCCCTGAAACTCTCCTGTGCAGCCTC
TGGATTCACTTTCAGTAGCTATGCCATGTCTTGGGTTCGCCATACTCCGGAGAAGAGGC
TGGAGTGGGTCGCAACCATTAGTAGTGGTGGTGGTTACACCTACTATCCAGACAGTGTG
AAGGGTCGATTCACCATCTCCAGAGACAATGCCAACAACATCCTGCACCTGCAAATGAG
CAGTCTGAGGTCTGAGGACACGGCCATGTATTACTGTACAAGAGGGGAGGGACTGGGAC
GAGCTATGGACTACTGGGGTCAAGGAACCTCAGTCACCGTCTCCTCAgacaagcagctg
gacgccgacgtgagccccaagcctaccatcttcctgcccagcatcgccgagaccaagct
gcagaaggccggcacctacctgtgcctgctggaaaagttcttccccgacgtgatcaaga
tccactggCaggaaaagaagagcaacaccatcctgggcagccaggaaggcaataccatg
aaaaccaacgacacctacatgaagttcagctggctgaccgtgcccgagaagagcctgga
caaagagcacagatgcatcgtccggcacgagaacaacaagaacggcgtggaccaggaaa
tcatcttcccccccatcaagaccgatgtgatcacaatggaTcccaaggacaactgcagc
aaggacgccaacgataccctgctgctgcagctgaccaacaccagcgcctactacatgta
tctcctgctgctgctgaagagcgtggtgtacttcgccatcatcacctgctgtctgctgc
ggcggaccgccttctgctgcaacggcgagaagagctag

The sequence of the CD8alpha subunit leader sequence is indicated in italic, bold lowercase font. The sequence of the mouse anti-human IgG1 variable Kappa light chain domain is indicated in bold uppercase font. The sequence of the human TCR delta constant domain is indicated in lowercase font. The sequence of a SGSG spacer and F2A is indicated in italic lowercase font. The sequence of the mouse anti-human IgG1 variable heavy chain domain is indicated in uppercase font. The sequence of the human TCR delta constant domain is indicated in bold lowercase font.

### B. GUAR-T Cells

In some forms, polypeptides, nucleic acids, or vectors encoding GUAR polypeptides are present within a host cell. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques.

Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell) can be used to, for example, produce the GUAR polypeptides described herein.

In some forms, the cell is from an established cell line, or a primary cell. The term “primary cell,” refers to cells and cell cultures derived from a subject and allowed to grow in vitro for a limited number of passages, i.e. splitting, of the culture. In exemplary forms, the introduction of a transposase and the viral vector including a GUAR into a host cell is performed ex vivo. In some forms, the cell is a T cell, hematopoietic stem cell (HSC), macrophage, natural killer cell (NK), or dendritic cell (DC). For example, in some forms, the T cell is a CD8+ T cell selected from the group including effector T cells, memory T cells, central memory T cells, and effector memory T cells. In other forms, the T cell is a CD4+ T cell selected from the group including Thl cells, Th2 cells, Th17 cells, and Treg cells. In some forms, the cell is an alpha-beta T cell that has been genetically modified to remove or diminish the expression of one or more expression products that are expressed in the wild-type cell.

### 1. Human Cells

In some forms, cells are obtained from a human subject. For example, in some forms, the cells are autologous cells, i.e., cells obtained from a subject prior to introduction of the GUAR polypeptides, and/or nucleic acids, or vectors encoding GUAR polypeptides, and re-introduction to the same subject following modification. In other forms, the cells are heterologous cells, i.e., cells obtained from a different subject than the intended recipient. In some forms, the cells are frozen prior to or after introduction of the GUAR polypeptides, and/or nucleic acids, or vectors encoding GUAR polypeptides. Methods and compositions for freezing and thawing viable eukaryotic cells are known in the art. In some forms, the cells are autologous immune cells, such as T cells or progenitor cells/stem cells.

In some forms, cells are obtained from a healthy subject. In other forms, cells are obtained from a subject identified as having or at risk of having a disease or disorder, such as cancer and/or an auto-immune disease.

In preferred forms, the introduction of the GUAR polypeptides to the cells occurs through genetic modification of the cells. In some forms, genetic modification of the cell includes introduction of nucleic acids, or vectors encoding GUAR polypeptides to the cell for expression of the GUAR polypeptides within the cell and presentation at the cell surface. In some forms, genetic modification of the cell includes transduction with a transposon encoding a GUAR polypeptide. In an exemplary form, a GUAR peptide is introduced into a cell in vitro by transduction of the cell with a nucleic acid encoding a transposon including the GUAR. Therefore, genetically modified (Transgenic) cells including GUAR proteins according to the described compositions are described. In some forms, T cells are engineered for endogenous gene editing on genes such as TRAC, TRBC, B2M and CIITA.

a. T Cells

In some forms, the cells are human immune cells, such as T cells. Therefore, in some forms, prior to expansion and genetic modification, T cells are obtained from a diseased or healthy subject. In some forms, the GUAR-T cells are autologous T cells obtained from a subject prior to ex vivo genetic modification (i.e., to express GUAR) and re-introduction to the same subject in vivo as GUAR T-cells.

T cells can be obtained from a number of samples, including peripheral blood mononuclear cells, bone marrow, lymph node tissue, cord blood, thymus tissue, tissue from a site of infection, ascites, pleural effusion, spleen tissue, and tumors. In some forms, T cells are obtained from a unit of blood collected from a subject using any number of techniques known to the skilled artisan, such as FICOLL™ separation. In one preferred form, cells from the circulating blood of an individual are obtained by apheresis. The apheresis product typically contains lymphocytes, including T cells, monocytes, granulocytes, B cells, other nucleated white blood cells, red blood cells, and platelets. The cells collected by apheresis can be washed to remove the plasma fraction and to place the cells in an appropriate buffer or media for subsequent processing steps. In some forms, the cells are washed with phosphate buffered saline (PBS). In some forms, the wash solution lacks calcium and can lack magnesium or can lack many if not all divalent cations. After washing, the cells can be resuspended in a variety of biocompatible buffers, such as, for example, Ca2+-free, Mg2+-free PBS, PLASMALYTE A, or other saline solution with or without buffer. Alternatively, the undesirable components of the apheresis sample are removed and the cells directly resuspended in culture media.

In some forms, T cells are isolated from peripheral blood lymphocytes by lysing the red blood cells and depleting the monocytes, for example, by centrifugation through a PERCOLL™ gradient or by counterflow centrifugal elutriation. In specific forms, a specific subpopulation of T cells, such as CD3+, CD28+, CD4+, CD8+, CD45RA+, and CD45RO+ T cells, is further isolated by positive or negative selection techniques. For example, in some forms, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. Therefore, T cells expressing heterologous GUAR at the surface of the T cells are provided.

In certain forms, the T cells are genetically modified T cells. For example, in some forms, the T cells are genetically modified to reduce, prevent or otherwise alter the expression of one or more genes within the “wild-type” T cell, in addition to the inclusion and expression of the GUAR within the same T cell. In some forms, the T cell is modified to reduce or prevent expression of one or more surface receptors that may interfere with the function or structure of the GUAR. For example, in some forms, the T cell is modified by silencing of one or more genes such as TCR alpha and/or TCR beta genes, i.e., to prevent expression of alpha-beta TCR proteins at the surface of GUAR-T cells. In some forms, the TRAC gene is targeted for removal or ablation. In certain forms, the host cells are Jurkat cells. Therefore, in some forms, the T cell is a genetically modified Jurkat cell that expresses GUAR at the surface under the control of the TCR alpha promoter. In some forms, the T cell does not express alpha-beta TCRs at the surface. Typically, the GUAR T-cell is capable, upon removal of a masking moiety and recognition of human monoclonal antibody, of degranulation and target-specific cell killing in vivo.

b. Delivery Vehicles

Any of the disclosed compositions including, but not limited to GUAR proteins, and/or nucleic acids, can be delivered to target cells using a delivery vehicle. The delivery vehicles can be, for example, polymeric particles, inorganic particles, silica particles, liposomes, micelles, multilamellar vesicles, etc.

Delivery vehicles may be microparticles or nanoparticles. Nanoparticles are often utilized for inter-tissue application, penetration of cells, and certain routes of administration. The nanoparticles may have any desired size for the intended use. The nanoparticles may have any diameter from 10 nm up to about 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In some forms the nanoparticles can have a diameter less than 400 nm, less than 300 nm, or less than 200 nm. The range can be between 50 nm and 300 nm.

Thus, in some forms, the delivery vehicles are nanoscale compositions, for example, 10 nm up to, but not including, about 1 micron. However, it will be appreciated that in some forms, and for some uses, the particles can be smaller, or larger (e.g., microparticles, etc.). Although many of the compositions disclosed herein are referred to as nanoparticle or nanocarrier compositions, it will be appreciated that in some forms and for some uses the carrier can be somewhat larger than nanoparticles. Such compositions can be referred to as microparticulate compositions. For example, a nanocarriers according to the present disclosure may be a microparticle. Microparticles can a diameter between, for example, 0.1 and 100 μm in size.

### C. Human Monoclonal Antibodies

In some forms, the compositions include human monoclonal antibodies directed against a known, specific target. An exemplary target is a cancer cell. The described GUAR molecules are configured to selectively and specifically bind to the conserved (Fc) component of human immunoglobulin G molecules. Therefore, GUAR-T cells recognize and activate upon binding to human IgG monoclonal antibodies in vivo. The presence or absence of a masking moiety in the region of the antigen-binding component of eth GUAR will typically control the antigen-binding and cell-killing activity of GUAR-T cells. Since the GUAR-T cells activate in response to recognition of any cell to which a human IgG molecule is bound, compositions of human monoclonal antibodies are provided. Generally, the human monoclonal antibodies (hmAbs) provide the targeting specificity for the desired GUAR T-cell activity in vivo. Therefore, compositions including GUAR-T cells and hmAbs having a specific and known antigen-binding activity are provided for targeted activity of GUAR-T cells. Since the GUAR T cells typically include masking moieties to prevent off-target cell killing and toxicity in vivo, the choice of hmAb is typically dependent upon the presence and type of masking moiety that is associated with the GUAR. For example, in some forms, where cancer cells are targeted, an anti-cancer cell-specific hmAb is chosen and the masking moiety on the GUAR is configured for release specifically in the presence of the tumor microenvironment. Therefore, in some forms, the antibody is a hmAb and the GUAR includes a making moiety having a urokinase-cleavable linker.

Any hmAb known in the art can be used in the described compositions. In some forms, the hmAb is a human cancer-specific hmAb. In some forms, the hmAb is a commercially available hmAb. Exemplary commercially available hmAbs include therapeutic hmAbs. Exemplary therapeutic hmAbs include FDA approved therapeutic monoclonal antibodies which include, but are not limited to, ACTEMRA® (tocilizumab, GENENTECH), ADCETRIS® (brentuximab vedotin, SEATTLE GENETICS), AMJEVITA® (adalimumab-atto, AMGEN INC), ANTHIM® (obiltoxaximab, ELUSYS THERAPEUTICS INC), ARZERRA® (ofatumumab, GLAXO GRP LTD), AVASTIN® (bevacizumab, GENENTECH), BAVENCIO® (avelumab, EMD SERONO INC), BENLYSTA® (belimumab, HUMAN GENOME SCIENCES INC.), BESPONSA® (inotuzumab ozogamicin, WYETH PHARMS INC), BLINCYTO® (blinatumomab, AMGEN), CAMPATH® (alemtuzumab, GENZYME), CIMZIA® (certolizumab pegol, UCB INC), CINQAIR® (reslizumab, TEVA RESPIRATORY LLC), COSENTYX® (secukinumab, NOVARTIS PHARMS CORP), CYLTEZO® (adalimumab-adbm, BOEHRINGER INGELHEIM), CYRAMZA® (ramucirumab, ELI LILLY AND CO), DARZALEX® (daratumumab, JANSSEN), DERMABET® (betamethasone valerate, TARO), DUPIXENT® (dupilumab, REGENERON PHARMACEUTICALS), EMPLICITI® (elotuzumab, BRISTOL MYERS SQUIBB), ENTYVIO® (vedolizumab, TAKEDA PHARMS USA), ERBITUX® (cetuximab, IMCLONE), FASENRA® (benralizumab, ASTRAZENECA AB), GAZYVA® (obinutuzumab, GENENTECH), HEMLIBRA® (emicizumab, GENENTECH INC), HERCEPTIN® (trastuzumab, GENENTECH), HUMIRA® (adalimumab, ABBVIE INC), ILARIS® (canakinumab, NOVARTIS PHARMS), ILUMYA® (tildrakizumab-asmn, MERCK SHARP DOHME), IMFINZI® (durvalumab, ASTRAZENECA UK LTD), INFLECTRA® (infliximab-dyyb, CELLTRION INC), IXIFI® (infliximab-qbtx, PFIZER INC), KADCYLA® (ado-trastuzumab emtansine, GENENTECH), KEVZARA® (sarilumab, SANOFI SYNTHELABO), KEYTRUDA® (pembrolizumab, MERCK SHARP DOHME), LARTRUVO® (olaratumab, ELI LILLY AND CO), LEMTRADA® (alemtuzumab, GENZYME), LUCENTIS® (ranibizumab, GENENTECH), MVASI® (bevacizumab-awwb, AMGEN INC), MYLOTARG® (gemtuzumab ozogamicin, WYETH PHARMS INC), MYOSCINT® (imciromab pentetate, CENTOCOR INC), NUCALA® (mepolizumab, GLAXOSMITHKLINE LLC), OCREVUS® (ocrelizumab, GENENTECH INC), OGIVRI® (trastuzumab-dkst, MYLAN GMBH), OPDIVO® (nivolumab, BRISTOL MYERS SQUIBB), PERJETA® (pertuzumab, GENENTECH), PORTRAZZA® (necitumumab, ELI LILLY CO), PRALUENT® (alirocumab, SANOFI AVENTIS), PRAXBIND® (idarucizumab, BOEHRINGER INGELHEIM), PROLIA® (denosumab, AMGEN), PROSTASCINT® (capromab pendetide, CYTOGEN), RAXIBACUMAB® (raxibacumab, HUMAN GENOME SCIENCES INC.), REMICADE® (infliximab, CENTOCOR INC), RENFLEXIS® (infliximab-abda, SAMSUNG BIOEPSIS CO LTD), REOPRO® (abciximab, CENTOCOR INC), REPATHA® (evolocumab, AMGEN INC), RITUXAN® (rituximab, GENENTECH), SILIQ® (brodalumab, VALEANT LUXEMBOURG), SIMPONI ARIA® (golimumab, JANSSEN BIOTECH), SIMULECT® (basiliximab, NOVARTIS), SOLIRIS® (eculizumab, ALEXION PHARM), STELARA® (ustekinumab, CENTOCOR ORTHO BIOTECH INC), STELARA® (ustekinumab, JANSSEN BIOTECH), SYLVANT® (siltuximab, JANSSEN BIOTECH), SYNAGIS® (palivizumab, MEDIMMUNE), TALTZ® (ixekizumab, ELI LILLY AND CO), TECENTRIQ® (atezolizumab, GENENTECH INC), TREMFYA® (guselkumab, JANSSEN BIOTECH), TROGARZO® (ibalizumab-uiyk, TAIMED BIOLOGICS USA), TYSABRI® (natalizumab, BIOGEN IDEC), UNITUXIN® (dinutuximab, UNITED THERAP), VECTIBIX® (panitumumab, AMGEN), XGEVA® (denosumab, AMGEN), XOLAIR® (omalizumab, GENENTECH), YERVOY® (ipilimumab, BRISTOL MYERS SQUIBB), ZEVALIN® (ibritumomab tiuxetan, SPECTRUM PHARMS), ZINBRYTA® (daclizumab, BIOGEN), ZINPLAVA® (bezlotoxumab, MERCK SHARP DOHME). In some forms, the hmAb is an anti-cancer therapeutic antibody. In some forms, the antibody is an anti-GPRC5D antibody, or an anti-HER2 antibody, or an anti-BCMA antibody.

### 1. Anti-GPRC5D Antibody

In some forms, the antibody specifically binds to a G protein-coupled receptor, class C, group 5, member D (GPRC5D). GPRC5D is an active immunotherapeutic target in multiple myeloma. Therefore, in some forms, the antibody is a human monoclonal anti-GPRC5D antibody.

B-cell maturation antigen (BCMA) is a member of the tumor necrosis factor receptor superfamily that plays an important role in regulating B-cell proliferation and survival. BCMA is expressed on the cell membrane of normal and malignant plasma cells, but not other normal tissues. Both BCMA and GPRC5D are prognostic factors in multiple myeloma (MM) patients. As demonstrated in the examples, high expression of BCMA and GPRC5D predicts unfavorable survival in newly diagnosed MM samples in; the combination of BCMA and GPRC5D could stratify patients with the shortest 2-year overall survival (OS) time, while BCMAhigh only and GPRC5D high only have better outcomes compared with BCMAhigh/GPRC5Dhigh patients.

An exemplary consensus amino acid sequence for the mature human monoclonal anti-GPRC5D antibody polypeptide variable heavy domain subunit is:

(SEQ ID NO: 17)
QVQLQQWGAGLLKPSETLSLTCAVYGGSFSGYYWSWIRQPPGKGLEWIG
EIIHSGSTNYNPSLKSRVTISVDTSKNQFSLKLSSVTAADTAVYYCARR
ITMVRGVIVNAFDIWGQGTMVTVSS.

In some forms, the mature anti-GPRC5D antibody variable heavy chain subunit polypeptide is a variant having at least 75%, up to 99% identity to SEQ ID NO:17. For example in some forms, the variant sequence has at least about 75%, 80%, 85%, 90%, or 95% identity to SEQ ID NO:17. Therefore, in some forms, the variant anti-GPRC5D antibody variable heavy chain subunit polypeptide has an amino acid sequence that has one or more amino acids different to SEQ ID NO:17, such as one or more substitutions, deletions or additions at any one of the amino acid positions of SEQ ID NO:17.

Typically, the variant is functional if it maintains the function of specific antigen binding. Therefore, the variant typically includes the three complementarity determining regions (CDRs 1-3) having an amino acid sequence of:

CDR-H1:
(SEQ ID NO: 21)
GYYWS;
CDR-H2:
(SEQ ID NO: 22)
EIIHSGSTNYNPSLKS;
and
CDR-H3:
(SEQ ID NO: 23)
RITMVRGVIVNAFDI.

An exemplary consensus amino acid sequence for the mature human monoclonal anti-GPRC5D antibody constant heavy1/constant heavy 2 domain (e.g., IgG-CH1-CH3) polypeptide is:

(SEQ ID NO: 18)
ASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK.

In some forms, the mature anti-GPRC5D antibody constant heavy1/constant heavy 2 domain polypeptide is a variant having at least 75%, up to 99% identity to SEQ ID NO:18. For example in some forms, the variant sequence has at least about 75%, 80%, 85%, 90%, or 95% identity to SEQ ID NO:18. Therefore, in some forms, the variant anti-GPRC5D antibody constant heavy1/constant heavy 2 domain polypeptide has an amino acid sequence that has one or more amino acids different to SEQ ID NO:18, such as one or more substitutions, deletions or additions at any one of the amino acid positions of SEQ ID NO:18. In some embodiments, there is little or no variation in the CDRs.

An exemplary consensus amino acid sequence for the mature human monoclonal anti-GPRC5D antibody variable light (kappa) domain polypeptide is:

(SEQ ID NO: 19)
DIQMTQSPSSLSASVGDRVSITCQASQDISHYLNWYQQKPGKAPKLLIYDASNLETGVP
SRFSGGGSGTDFTFTISSLQPADIATYYCQQYDHLPYTFGQGTKLEIKR.

In some forms, the mature anti-GPRC5D antibody variable light (kappa) domain polypeptide is a variant having at least 75%, up to 99% identity to SEQ ID NO:19. For example in some forms, the variant sequence has at least about 75%, 80%, 85%, 90%, or 95% identity to SEQ ID NO:19. Therefore, in some forms, the variant anti-GPRC5D antibody variable light (kappa) domain polypeptide has an amino acid sequence that has one or more amino acids different to SEQ ID NO:19, such as one or more substitutions, deletions or additions at any one of the amino acid positions of SEQ ID NO:19.

Typically, the variant is functional if it maintains the function of specific antigen binding. Therefore, the variant typically includes the three complementarity determining regions (CDRs 1-3) having an amino acid sequence of:

CDR-L1:
(SEQ ID NO: 24)
QASQDISHYLN;
CDR-L2:
(SEQ ID NO: 25)
DASNLET;
and
CDR-L3:
(SEQ ID NO: 26)
QQYDHLPYT.

An exemplary consensus amino acid sequence for the mature human monoclonal anti-GPRC5D antibody constant light (kappa) domain polypeptide is:

(SEQ ID NO: 20)
TVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC.

In some forms, the mature anti-GPRC5D antibody constant light (kappa) domain polypeptide is a variant having at least 75%, up to 99% identity to SEQ ID NO:20. For example in some forms, the variant sequence has at least about 75%, 80%, 85%, 90%, or 95% identity to SEQ ID NO:20. Therefore, in some forms, the variant anti-GPRC5D antibody constant light (kappa) domain polypeptide has an amino acid sequence that has one or more amino acids different to SEQ ID NO:20, such as one or more substitutions, deletions or additions at any one of the amino acid positions of SEQ ID NO:20. In some embodiments, there is little or no variation in the CDRs.

Antibodies and antigen binding polypeptides thereof including the CDRs of SEQ ID NO:19 and/or SEQ ID NO:17 are also provided and can be used together or separately from other elements of the disclosed compositions. Thus, antibodies and antigen binding polypeptides including the CDRs of SEQ ID NO:19 (e.g., SEQ ID NOS:24, 25, and 26) containing within a light chain variable region framework and/or including the CDRs of SEQ ID NO:17 (e.g., SEQ ID NOS:21, 22, and 23) contained within a heavy chain variable region framework are provided. For example, the CDRs of SEQ ID NO:19 can be presented in a different light chain variable region framework and/or the CDRs of SEQ ID NO:17 can be presented in a different heavy chain variable region framework, e.g., a humanized or chimeric framework. Typically the CDRs are presented in the same orientation as in SEQ ID NO:19 and/or SEQ ID NO:17 (e.g., CDR-L1 is SEQ ID NO:24, CDR-L2 is SEQ ID NO:25, and CDR-L3 is SEQ ID NO:26; and/or CDR-H1 is SEQ ID NO:21, CDR-H2 is SEQ ID NO:22, and CDR-H3 is SEQ ID NO:23). The antibodies and antigen binding polypeptides can be full antibodies or fragments or synthetic fusion polypeptides that bind the target antigen. The antibodies and antigen binding polypeptides can be humanized or chimeric. Exemplary fragments and fusions include, but are not limited to, single chain antibodies, single chain variable fragments (scFv), disulfide-linked Fvs (sdFv), Fab′, F(ab′)2, Fv, and single domain antibody fragments (sdAb). In some embodiments, the molecule includes two or more scFv.

A humanized or chimeric antibody can include substantially all of at least one, and typically two, variable domains in which all or substantially all of the CDR regions correspond to those of a non-human immunoglobulin (i.e., donor antibody) and all or substantially all of the framework regions are those of a human immunoglobulin consensus sequence. Preferably, the antibody also includes at least a portion of an immunoglobulin constant region (Fc), typically that of a human immunoglobulin. The constant domains of the antibodies may be selected with respect to the proposed function of the antibody, in particular the effector function which may be required. In some embodiments, the constant domains of the antibodies are (or include) human IgA, IgD, IgE, IgG or IgM domains. In a specific embodiment, human IgG constant domains, especially of the IgG1 and IgG3 isotypes are used, when the humanized antibody is intended for therapeutic uses and antibody effector functions such as antibody-dependent cell-mediated cytotoxicity (ADCC) and/or complement-dependent cytotoxicity (CDC) activity are needed. In alternative embodiments, IgG2 and IgG4 isotypes are used when the antibody is intended for therapeutic purposes and antibody effector function is not required. The antibodies can include Fc constant domains including one or more amino acid modifications which alter antibody effector functions such as those disclosed in U.S. Patent Application Publication Nos. 2005/0037000 and 2005/0064514.

In some embodiments, the antibody contains both at least the variable domain of a light chain as well as at least the variable domain of a heavy chain. In other embodiments, the antibody may further include one or more of the CL of the light chain, and/or one of more of the CH1, hinge, CH2, CH3, and CH4 regions of the heavy chain. The antibody can be selected from any class of immunoglobulins, including IgM, IgG, IgD, IgA and IgE, and any isotype, including IgG1, IgG2, IgG3 and IgG4. In some embodiments, the constant domain is a complement fixing constant domain where it is desired that the antibody exhibit cytotoxic activity, and the class is typically IgG1. In other embodiments, where such cytotoxic activity is not desirable, the constant domain may be of the IgG2 class. The antibody may include sequences from more than one class or isotype, and selecting particular constant domains to optimize desired effector functions is within the ordinary skill in the art.

The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the donor antibody. Such mutations, however, are preferably not extensive. Usually, at least 75% of the humanized antibody residues will correspond to those of the parental framework region (FR) and CDR sequences, more often 90%, and most preferably greater than 95%. Humanized antibodies can be produced using variety of techniques known in the art, including, but not limited to, CDR grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805 814; and Roguska et al., 1994, Proc. Natl. Acad. Sci. 91:969 973), chain shuffling (U.S. Pat. No. 5,565,332), and techniques disclosed in, e.g., U.S. Pat. Nos. 6,407,213, 5,766,886, 5,585,089, International Publication No. WO 9317105, Tan et al., 2002, J. Immunol. 169:1119 25, Caldas et al., 2000, Protein Eng. 13:353 60, Morea et al., 2000, Methods 20:267 79, Baca et al., 1997, J. Biol. Chem. 272:10678 84, Roguska et al., 1996, Protein Eng. 9:895 904, Couto et al., 1995, Cancer Res. 55 (23 Supp):5973s 5977s, Couto et al., 1995, Cancer Res. 55:1717 22, Sandhu, 1994, Gene 150:409 10, Pedersen et al., 1994, J. Mol. Biol. 235:959 73, Jones et al., 1986, Nature 321:522-525, Riechmann et al., 1988, Nature 332:323, and Presta, 1992, Curr. Op. Struct. Biol. 2:593-596. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; U.S. Publication Nos. 2004/0049014 and 2003/0229208; U.S. Pat. Nos. 6,350,861; 6,180,370; 5,693,762; 5,693,761; 5,585,089; and 5,530,101 and Riechmann et al., 1988, Nature 332:323).

The antibodies used in the methods of the present invention may be monospecific. Also of interest are bispecific antibodies, tri-specific antibodies or antibodies of greater multi-specificity that exhibit specificity to different targets in addition to the target antigen. For example, such antibodies may bind to both the target antigen and to an antigen that is important for targeting the antibody to a particular cell type or tissue (for example, to an antigen associated with a cancer antigen of a tumor being treated) or to an immune cell. In another embodiment, such multi-specific antibody binds to molecules (receptors or ligands) involved in alternative or supplemental immunomodulatory pathways, such as CTLA4, TIM3, TIM4, OX40, CD40, GITR, 4-1-BB, CD27/CD70, ICOS, B7-H4, LIGHT, PD-1 or LAG3, in order to diminish further modulate the immunomodulatory effects. Furthermore, the multi-specific antibody may bind to effecter molecules such as cytokines (e.g., IL-7, IL-15, IL-12, IL-4 TGF-beta, IL-10, IL-17, IFNg, Flt3, BLys) and chemokines (e.g., CCL21), which may be particularly relevant for down-modulating both acute and chronic immune responses.

Any of the antibodies can be conjugated to a diagnostic or therapeutic agent (e.g., chemotherapeutic agent) or any other molecule for which serum half-life is desired to be increased. The antibodies can be used diagnostically (in vivo, in situ or in vitro) to, for example, monitor the development or progression of a disease, disorder or infection as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present invention.

### D. Exemplary Vectors

Also provided are exemplary vector nucleic acid sequences and their associated vector maps are provided below, in the associated Figures, and in the associated sequences listing. It will be appreciated that each of the vectors are provided as whole vector nucleic acid sequences and as component part subsequences that can be identified by e.g., the full sequence and the provided vector maps. Furthermore, the translated amino acid sequence of each of the polypeptides encoded by each of the vectors is also expressly individually provided, as are variants thereof with at least 50%, 60%, 70%, 75%, 80%, 85%, 90%, or 95% sequence identity thereto. Each of the full vector nucleic acid sequences, vector nucleic acid subsequences, and encoded polypeptide sequences can be further used as part of whole of the compositions provided herein and/or in the disclosed methods.
1. Vector GPRC5D-2-H (SEQ ID NO:34);
2. Vector GPRC5D-2-K (SEQ ID NO:35);
3. Vector pMB85b AAV6-TRAC-KI-EFS-GUART-ATX-Clone1 (SEQ ID NO:36);
4. Vector pMB85c AAV6-TRAC-KI-EFS-GUART-ATX-Clone2 (SEQ ID NO:37);
5. Vector pMB85d AAV6-TRAC-KI-EFS-GUART-ATX-Clone3 (SEQ ID NO:38);
6. Vector pMB99 AAV6-TRAC-KI-EFS-GUART-ATX-Clone2-CD28-OX40 (SEQ ID NO:39);
7. Vector pMB100 AAV6-TRAC-KI-EFS-GUART-ATX-Clone2-CD28-deltaIL2RBdna (SEQ ID NO:40);
8. Vector pMB101 AAV6-TRAC-KI-EFS-GUART-ATX-Clone2-CD28-ICOS (SEQ ID NO:41);
9. Vector pMB102 AAV6-TRAC-KI-EFS-GUART-ATX-Clone2-CD28-IL7RA (SEQ ID NO:42);
10. Vector pPR66 pFuse-human IgG1 (Glu99-Lys330, C103S) (SEQ ID NO:43);
11. Vector pPR116-pESC-human-anti-human IgG1-Clone1 (SEQ ID NO:44);
12. Vector pPR117-pESC-human-anti-human IgG1-Clone2 (SEQ ID NO:45);
13. Vector pPR118-pESC-human-anti-human IgG1-Clone3 (SEQ ID NO:46);
14. Vector pPR134 Lenti-EF1a-BCMA-Fab TCR Gamma-Delta WT (SEQ ID NO:47);
15. Vector pPR138 Lenti-EF1a-ATX-LgG1-Clone1-Fab TCR Gamma-Delta WT (SEQ ID NO:48);
16. Vector pPR139 Lenti-EF1a-ATX-IgG1-Clone2-Fab TCR Gamma-Delta WT (SEQ ID NO:49);
17. Vector pPR140 Lenti-EF1a-ATX-IgG1-Clone3-Fab-TCR Gamma-Delta WT (SEQ ID NO:50);
18. Vector pPR142 Lenti-EF1a-mouse-IgG1-Clone8-scfv-Fab TCR Gamma-Delta WT (SEQ ID NO:51);
19. Vector pPR143 Lenti-EF1a-mouse-IgG1-Clone8-scfv-scfv TCR Gamma-Delta WT (SEQ ID NO:52);
20. Vector pPR144 Lenti-EF1a-mouse-IgG1-Clone8-scfv-re-scfv TCR Gamma-Delta WT (SEQ ID NO:53);
21. Vector pPR145 Lenti-EF1a-mouse-IgG1-Clone8-clone18 TCR Gamma-Delta WT (SEQ ID NO:54);
22. Vector pPR155 Lenti-EF1a-CC3-ATX-Clone2-GUAR (SEQ ID NO:55);
23. Vector pPR156 Lenti-EF1a-CC4-ATX-Clone2-GUAR (SEQ ID NO:56);
24. Vector pPR157 Lenti-EF1a-CC2B-ATX-Clone2-GUAR (SEQ ID NO:57);
25. Vector pPR163 Lenti-EF1a-CC3-uPA-ATX-Clone2-GUAR (SEQ ID NO:58);
26. Vector pPR164 Lenti-EF1a-CC2B-uPAATX-Clone2-GUAR (SEQ ID NO:59);
27. Vector pPR171 Lenti-EF1a-CC3-uPA-linker-ATX-Clone2-GUAR (SEQ ID NO:60);
28. Vector pPR172 Lenti-EF1a-CC2B-uPA-linker-ATX-Clone2-GUAR (SEQ ID NO:61);
29. Vector pPR178 Lenti-EF1a-CC3-uPA-linker-ATX-Clone2-GUAR (SEQ ID NO:62);
30. Vector pPR179 Lenti-EF1a-CC3-uPA-linker-ATX-Clone2-GUAR (SEQ ID NO:63);
31. Vector pPR180 Lenti-EF1a-CC3-uPA-linker-ATX-Clone2-GUAR (SEQ ID NO:64);
32. Vector pPR181 Lenti-EF1a-CC3-uPA-linker-ATX-Clone2-GUAR (SEQ ID NO:65);
33. Vector pPR188 Lenti-EF1a-CC3-2uPA-ATX-Clone2-GUAR (SEQ ID NO:66);
34. Vector pPRI 89 Lenti-EF1a-CC2B-2uPA-ATX-Clone2-GUAR (SEQ ID NO:67);
35. Vector pPR190 Lenti-EF1a-CC3-uPA-MMP-ATX-Clone2-GUAR (SEQ ID NO:68);
36. Vector pPR191 Lenti-EF1a-CC2B-uPA-MMP-ATX-Clone2-GUAR (SEQ ID NO:69);
37. Vector pPR202 Lenti-EF1a-CC3-uPA-linker-ATX-Clone2-GUAR-G (SEQ ID NO:70);
38. Vector pPR203 Lenti-EF1a-CC3-uPA-linker-ATX-Clone2-GUAR-GG (SEQ ID NO:71);
39. Vector pPR204 Lenti-EF1a-CC3-uPA-linker-ATX-Clone2-GUAR-GGS (SEQ ID NO:72);
40. Vector pPR205 Lenti-EF1a-CC3-uPA-linker-ATX-Clone2-GUAR-GGGS (SEQ ID NO:73);
41. Vector pPR212 Lenti-EF1a-CC4-uPA-linker-ATX-Clone2-GUAR (SEQ ID NO:74);
42. Vector pPR213 Lenti-EF1a-CC5-uPA-linker-ATX-Clone2-GUAR (SEQ ID NO:75);
43. Vector pPR226 pFuse-human IgG1 (Glu99-Lys330, C103S) 139-1 mutant (SEQ ID NO:76);
44. Vector pPR227 pFuse-human IgG1 (Glu99-Lys330, C103S) 139-2 mutant (SEQ ID NO:77).

### E. Pharmaceutical Compositions

Pharmaceutical compositions containing a genetically modified cell, or a population of genetically modified cells expressing GUAR proteins, or compositions of human monoclonal immunoglobulins alone or together with genetically modified cells expressing GUAR proteins are provided.

In some forms, the pharmaceutical compositions include one or more of a pharmaceutically acceptable buffer, carrier, diluent or excipients. In some forms, the pharmaceutical compositions include a specific number or population of cells, for example, expanded by culturing and expanding an isolated genetically modified cell (e.g., GUAR T cell), e.g., a homogenous population. Therefore, in some forms, pharmaceutical compositions include a homogenous population of modified cells including and/or expressing a GUAR peptide. In other forms, the pharmaceutical compositions include populations of cells that contain variable or different genetically modified cells, e.g., a heterogeneous population. In some forms, the pharmaceutical compositions include cells that are bispecific or multi-specific. Any of the compositions can include one or more species of human monoclonal antibodies, for example, targeting a specific antigen to which the GUAR T-cells are intended to be targeted against. In other forms, the compositions include no cells, but include one or more species of human monoclonal antibodies, for example, targeting a specific antigen. In some forms, composition of antibodies to which the GUAR T-cells are intended to be targeted include one or more therapeutic antibodies. In some forms, the compositions include an anti-GPRC5D antibody. An exemplary anti-GPRC5D antibody has the amino acid sequence has a light chain variable region including the CDRs of SEQ ID NO:19 (optionally SEQ ID NOS:24-26) optionally the light chain variable region of SEQ ID NO:19 or a variant thereof with at least 70% sequence identity thereto; a heavy chain variable region including the CDRs of SEQ ID NO:17 (optionally SEQ ID NOS:21-23) optionally the heavy chain variable region of SEQ ID NO:17 or a variant thereof with at least 70% sequence identity thereto; or more preferably a combination thereof.

In some forms, the cells have been isolated from a diseased or healthy subject prior to genetic modification to express a GUAR peptide.

The term “pharmaceutically acceptable carrier” describes a pharmaceutically acceptable material, composition, or vehicle that is involved in carrying or transporting a compound of interest from one tissue, organ, or portion of the body to another tissue, organ, or portion of the body. For example, in some forms the carrier is a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In some forms, pharmaceutical compositions include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. The pharmaceutical compositions can be formulated for delivery via any route of administration. The term “Route of administration” can refer to any administration pathway known in the art, including but not limited to aerosol, nasal, oral, intravenous, intramuscular, intraperitoneal, inhalation, transmucosal, transdermal, parenteral, implantable pump, continuous infusion, topical application, capsules and/or injections. The pharmaceutical compositions are preferably formulated for intravenous administration.

Typically, the disclosed pharmaceutical compositions are administered in a manner appropriate to a disease to be treated (or prevented). The quantity and frequency of administration is typically determined by such factors as the condition of the patient, and the type and severity of the patient's disease, although appropriate dosages can be determined by clinical trials.

The disclosed pharmaceutical compositions can be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### III. Methods

Methods of using compositions including GUAR proteins and/or GUAR T-cells and/or human monoclonal antibodies are provided.

In particular forms, the methods provide enhanced anti-tumor activity through administration of GUAR-T cells. It has been established that GUAR T-cells provide highly selective, readily diversifiable targeting of specific cells in vivo through co-administration of human monoclonal immunoglobulins specific for the target cell-type in vivo. The features of GUAR T-cell therapy were developed into a simple yet versatile approach to enhance engineered T cell activities by readily changing the target through changing the co-administered mAb. As set forth within experimental data in the examples, GUAR T cells, exhibit highly-specific cytolysis against mAb-targeted cognate cancer cells. Methods of treatment of a subject for a disease include administering GUAR T-cells to the subject.

### A. Methods of Treatment

Methods of treatment including cells and other therapeutic agents including GUAR polypeptides, and optionally further including human monoclonal antibodies are described. In preferred forms, the methods include Adoptive Cell Therapy (ACT) employing T cells expressing recombinant GUAR proteins (GUAR T-cells). The GUAR T-cells administered together with anti-target cell monoclonal antibodies have anti-target cell immune activity.

An exemplary method involves treating a subject (e.g., a human) having a disease, disorder, or condition associated with the presence or proliferation of undesired cells by administering to the subject an effective amount of a pharmaceutical composition including genetically-modified cells including GUAR polypeptides and optionally monoclonal antibodies specific for the undesired cell (i.e., target cell). In some forms, the methods administer genetically manipulated T cells engineered to express recombinant GUAR proteins and optionally monoclonal antibodies to a subject (e.g., a human) having a disease, disorder, or condition in an amount effective to treat the disease, disorder, or condition. For example, in some forms, the methods treat a disease or disorder associated with an elevated expression or specific expression of an antigen by administering to the subject an effective amount of a pharmaceutical composition including cells modified to express GUAR proteins and optionally monoclonal antibodies specific for the undesired cell (i.e., target cell).

In some forms, the methods treat a subject having a disease, disorder, or condition associated with an elevated expression or specific expression of an antigen by administering to the subject an effective amount of a pharmaceutical composition including T cells modified to contain a GUAR that targets the antigen. In some forms, the disease, disorder, or condition associated with an elevated expression or specific expression of an antigen is cancer, and the antigen is a cancer antigen. Therefore, methods of using GUAR T-cells, optionally together with a human monoclonal antibody specific for a cancer antigen are provided.

Methods of using pharmaceutical compositions of GUAR T-cells alone, or together with pharmaceutical compositions of a human monoclonal antibody to treat a disease or disorder by are provided. Typically the methods include ACT, for example, by providing GUAR-bearing T cells with a human monoclonal antibody for therapeutic efficacy in vivo. In some forms, the methods of ACT including administering GUAR-T cells with a human monoclonal antibody have enhanced efficacy in vivo relative to ACT using conventional CAR-T cells.

Methods of treating a subject having a disease, disorder, or condition including administering to the subject an effective amount of a pharmaceutical composition including live, viable cells engineered to express GUAR proteins are provided. In some forms, when the methods treat a subject having a disease, disorder, or condition associated with an elevated expression or specific expression of an antigen, the methods include administering to the subject an effective amount of a T cell modified to express a GUAR together with an effective amount of a human monoclonal antibody that targets the antigen. For example, in some forms, the methods treat a subject having a disease, disorder, or condition associated with an elevated expression or specific expression of an antigen by administering to the subject an effective amount of a pharmaceutical composition having a genetically modified cell, where the cell is modified by introducing to the cell:
(i) nucleic acid, e.g., a DNA or RNA such as viral RNA or mRNA, optionally, but preferably a vector, optionally including a transposon encoding a GUAR protein; and
(ii) causing the GUAR protein to be expressed by the cell.

The cell can have been isolated from the subject having the disease, disorder, or condition, or from a healthy donor, prior to genetic modification. In some forms, the methods also include administering to the subject an effective amount of a monoclonal antibody, e.g., human monoclonal antibody, that targets the antigen. The hmAb can target an antigen selected from a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.

### 1. Diseases to be Treated

Methods of treating diseases and/or disorders in a subject in need thereof are provided. The subject to be treated can have a disease, disorder, or condition such as but not limited to, cancer, an immune system disorder such autoimmune disease, an inflammatory disease, a neuronal disorder, HIV/AIDS, diabetes, a cardiovascular disease, an infectious disease, or combinations thereof. The disease, disorder, or condition can be associated with an elevated expression or specific expression of an antigen.

a. Cancer

In some forms, the methods treat or prevent cancer. In some forms, the methods treat or prevent cancer or other proliferative disease or disorder in a subject identified as having, or at risk of having cancer or other proliferative disease or disorder. Cancer is a disease of genetic instability, allowing a cancer cell to acquire the hallmarks proposed by Hanahan and Weinberg, including (i) self-sufficiency in growth signals; (ii) insensitivity to anti-growth signals; (iii) evading apoptosis; (iv) sustained angiogenesis; (v) tissue invasion and metastasis; (vi) limitless replicative potential; (vii) reprogramming of energy metabolism; and (viii) evading immune destruction (Cell., 144:646-674, (2011)).

Tumors, which can be treated in accordance with the disclosed methods, are classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

TABLE 1
Exemplary cancers for which the GUAR T-cells together with antigen-
specific monoclonal antibodies targeting a cancer antigen of the disclosed methods and
compositions can target a specific or an associated antigen.

| | | | | |
|---|---|---|---|---|
| Acute | Acute Myeloid | Adrenocortical | AIDS-Related | Kaposi Sarcoma |
| Lymphoblastic | Leukemia | Carcinoma | Cancers | |
| Leukemia | (AML) | | | |
| (ALL) | | | | |
| AIDS-Related | Primary CNS | Anal Cancer | Appendix | Astrocytomas |
| Lymphoma | Lymphoma | | Cancer | |
| | | | (Gastrointestinal | |
| | | | Carcinoid | |
| | | | Tumors) | |
| Atypical | Brain Cancer | Basal Cell | Bile Duct | Bladder Cancer |
| Teratoid/ | | Carcinoma of the | Cancer | |
| Rhabdoid | | Skin | | |
| Tumor | | | | |
| Bone Cancer | Brain Tumors | Breast Cancer | Bronchial | Burkitt |
| (includes | | | Tumors | Lymphoma |
| Ewing Sarcoma | | | | |
| and | | | | |
| Osteosarcoma | | | | |
| and Malignant | | | | |
| Fibrous | | | | |
| Histiocytoma) | | | | |
| Non-Hodgkin | Carcinoid | Carcinoma of | Cardiac (Heart) | Embryonal |
| Lymphoma | Tumors | Unknown Primary | Tumors | Tumors |
| Germ Cell | Primary CNS | Cervical Cancer | Cholangio- | Chordoma |
| Tumor | Lymphoma | | carcinoma | |
| Chronic | Chronic | Chronic | Colorectal | Cranio- |
| Lymphocytic | Myelogenous | Myeloproliferative | Cancer | pharyngioma |
| Leukemia | Leukemia | Neoplasms | | |
| (CLL) | (CML) | | | |
| Cutaneous T- | Ductal | Endometrial Cancer | Ependymoma | Esophageal |
| Cell | Carcinoma In | | | Cancer |
| Lymphoma | Situ (DCIS) | | | |
| (Mycosis | | | | |
| Fungoides and | | | | |
| Sézary | | | | |
| Syndrome) | | | | |
| Esthesioneuro- | Ewing Sarcoma | Extracranial Germ | Eye Cancer | Intraocular |
| blastoma | | Cell Tumor | | Melanoma |
| Fallopian Tube | Fibrous | Osteosarcoma | Gallbladder | Gastric Cancer |
| Cancer | Histiocytoma of | | Cancer | |
| | Bone | | | |
| Stomach | Gastrointestinal | Gastrointestinal | Central | Extracranial |
| Cancer | Carcinoid | Stromal Tumors | Nervous | Germ Cell |
| | Tumor | (GIST) | System Germ | Tumors |
| | | | Cell Tumors | |
| Extragonadal | Ovarian Germ | Testicular Cancer | Gestational | Hairy Cell |
| Germ Cell | Cell Tumors | | Trophoblastic | Leukemia |
| Tumors | | | Disease | |
| Head and Neck | Heart Tumors | Hepatocellular | Histiocytosis | Hodgkin |
| Cancer | | (Liver) Cancer | (Langerhans | Lymphoma |
| | | | Cell) | |
| Hypopharyngeal | Intraocular | Islet Cell Tumors | Pancreatic | Kidney Cancer |
| Cancer | Melanoma | | Neuroendocrine | |
| | | | Tumors | |
| Renal Cell | Langerhans | Laryngeal Cancer | Leukemia | Lip and Oral |
| Cancer | Cell | | | Cavity Cancer |
| | Histiocytosis | | | |
| Liver Cancer | Lung Cancer | Lymphoma | Male Breast | Malignant |
| | (Non-Small | | Cancer | Fibrous |
| | Cell and Small | | | Histiocytoma of |
| | Cell) | | | Bone and |
| | | | | Osteosarcoma |
| Melanoma | Intraocular | Merkel Cell | Malignant | Metastatic |
| | (Eye) | Carcinoma (Skin | Mesothelioma | Cancer |
| | Melanoma | Cancer) | | |
| Metastatic | Midline Tract | Mouth Cancer | Multiple | Multiple |
| Squamous | Carcinoma | | Endocrine | Myeloma/Plasma |
| Neck Cancer | With NUT | | Neoplasia | Cell |
| with Occult | Gene Changes | | Syndromes | Neoplasms |
| Primary | | | | |
| Mycosis | Myelodysplastic | Myelodysplastic/ | Nasal Cavity | Nasopharyngeal |
| Fungoides | Syndromes | Myeloproliferative | and Paranasal | Cancer |
| (Lymphoma) | | Neoplasms | Sinus Cancer | |
| Neuroblastoma | Non-Small Cell | Oral Cancer | Oropharyngeal | Ovarian Cancer |
| | Lung Cancer | | Cancer | |
| Pancreatic | Papillomatosis | Paraganglioma | Paranasal Sinus | Parathyroid |
| Cancer | | | and Nasal | Cancer |
| | | | Cavity Cancer | |
| Penile Cancer | Pharyngeal | Pheochromocytoma | Pituitary | Plasma Cell |
| | Cancer | | Tumor | Neoplasm/ |
| | | | | Multiple |
| | | | | Myeloma |
| Pleuropulmonary | Primary Central | Primary Peritoneal | Prostate Cancer | Rectal Cancer |
| Blastoma | Nervous | Cancer | | |
| | System (CNS) | | | |
| | Lymphoma | | | |
| Recurrent | Retinoblastoma | Rhabdomyosarcoma | Salivary Gland | Sarcoma |
| Cancer | | | Cancer | |
| Vascular | Uterine | Sézary Syndrome | Small Cell | Small Intestine |
| Tumors | Sarcoma | (Lymphoma) | Lung Cancer | Cancer |
| Soft Tissue | Squamous Cell | Stomach (Gastric) | Throat Cancer | Thymoma |
| Sarcoma | Carcinoma | Cancer | | |
| Thymic | Thyroid Cancer | Transitional Cell | Carcinoma of | Ureter and |
| Carcinoma | | Cancer of the Renal | Unknown | Renal Pelvis |
| | | Pelvis and Ureter | Primary | |
| Transitional | Urethral Cancer | Uterine Cancer | Vaginal Cancer | Vulvar Cancer |
| Cell Cancer | | | | |
| Wilms Tumor | | | | |

The disclosed compositions and methods can be used in the treatment of one or more cancers provided in Table 1.

The disclosed compositions and methods of treatment thereof are generally suited for treatment of carcinomas, sarcomas, lymphomas and leukemias. The described compositions and methods are useful for treating, or alleviating subjects having benign or malignant tumors by delaying or inhibiting the growth/proliferation or viability of tumor cells in a subject, reducing the number, growth or size of tumors, inhibiting or reducing metastasis of the tumor, and/or inhibiting or reducing symptoms associated with tumor development or growth.

The types of cancer that can be treated with the provided compositions and methods include, but are not limited to, cancers such as vascular cancer such as multiple myeloma, adenocarcinomas and sarcomas, of bone, bladder, brain, breast, cervical, colorectal, esophageal, kidney, liver, lung, nasopharyngeal, pancreatic, prostate, skin, stomach, and uterine. The experiments below support the conclusion that the disclosure approach is effective for treating solid tumors. Thus, in some embodiments, the target cancer is a solid tumor. In some forms, the compositions are used to treat multiple cancer types concurrently. The compositions can also be used to treat metastases or tumors at multiple locations.

Exemplary tumor cells include, but are not limited to, tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as, but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytoma and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipidus; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or ureter); Wilms' tumor; bladder cancers including, but not limited to, transitional cell carcinoma, squamous cell cancer, adenocarcinoma, and carcinosarcoma. For a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

### 2. Immune System Disorders

In some forms, the methods administer modified T cells including GUAR-protein(s), optionally together with one or more monoclonal antibodies to treat or prevent one or more immune system disorders, including autoimmune diseases.

Under certain circumstances, the ability of the immune system to distinguish self from foreign antigens can be misdirected against healthy tissues, resulting in the undesirable attack and destruction of normal host cells (i.e., autoimmune diseases). Autoimmune diseases include over 100 types of diseases, with varied etiology and prognoses based on factors such as the affected region, the age of onset, response to the therapeutic agents and clinical manifestation may vary among different people (Muhammad, et al., Chimeric Antigen Receptor Based Therapy as a Potential Approach in Autoimmune Diseases: How Close Are We to the Treatment, Frontiers in Immunology, 11 (2020)).

Auto-antibody-secreting B lymphocytes and self-reactive T-lymphocytes play a key role in the development of autoimmune diseases. Based on the extent of tissue damage, autoimmunity is classified into two general categories, including organ-specific and systemic autoimmune. The former involves a specific area of the body such as type I diabetes (T1D), multiple sclerosis (MS), rheumatoid arthritis (RA), inflammatory bowel diseases (IBDs), and myasthenia gravis (MG), while the latter affects multiple regions of the body, causing systemic lupus erythematosus (SLE) and Sjögren's syndrome (SS). Therefore, in some forms, the methods treat or prevent one or more organ-specific autoimmune diseases in a subject. In other forms, the methods treat or prevent one or more systemic autoimmune diseases in a subject.

In some forms, the methods reduce or prevent one or more physiological processes associated with the development or progression of autoimmune disease in a subject. For example, in some forms, the methods reduce or prevent one or more of epitope spreading, for example, where infections alter the primary epitope into the secondary epitope or form several neoepitopes on antigen-presenting cells; bystander activation or pre-primed autoreactive T cell activation in a T cell receptor (TCR)-independent manner; persistent virus infection, or the constant presence of viral antigens that prompt immune responses; or immunological cross-reactivity between a host and pathogen, for example, due to shared immunologic epitopes or sequence similarities.

In some forms, the methods administer a GUAR T-cell together with a monoclonal antibody targeting a molecule or receptor on a cell that is associated with, responsible for or otherwise developing or maintaining an immune system disease or disorder. Non-limiting examples of immune system disorders that can be treated or prevented by the methods include 22q11.2 deletion syndrome, Achondroplasia and severe combined immunodeficiency, Adenosine Deaminase 2 deficiency, Adenosine deaminase deficiency, Adult-onset immunodeficiency with anti-interferon-gamma autoantibodies, Agammaglobulinemia, non-Bruton type, Aicardi-Goutieres syndrome, Aicardi-Goutieres syndrome type 5, Allergic bronchopulmonary aspergillosis, Alopecia, Alopecia totalis, Alopecia universalis, Amyloidosis AA, Amyloidosis familial visceral, Ataxia telangiectasia, Autoimmune lymphoproliferative syndrome, Autoimmune lymphoproliferative syndrome due to CTLA4 haploinsuffiency, Autoimmune polyglandular syndrome type 1, Autosomal dominant hyper IgE syndrome, Autosomal recessive early-onset inflammatory bowel disease, Autosomal recessive hyper IgE syndrome, Bare lymphocyte syndrome 2, Barth syndrome, Blau syndrome, Bloom syndrome, Bronchiolitis obliterans, C1q deficiency, Candidiasis familial chronic mucocutaneous, autosomal recessive, Cartilage-hair hypoplasia, CHARGE syndrome, Chediak-Higashi syndrome, Cherubism, Chronic atypical neutrophilic dermatosis with lipodystrophy and elevated temperature, Chronic graft versus host disease, Chronic granulomatous disease, Chronic Infantile Neurological Cutaneous Articular syndrome, Chronic mucocutaneous candidiasis (CMC), Cohen syndrome, Combined immunodeficiency with skin granulomas, Common variable immunodeficiency, Complement component 2 deficiency, Complement component 8 deficiency type 1, Complement component 8 deficiency type 2, Congenital pulmonary alveolar proteinosis, Cryoglobulinemia, Cutaneous mastocytoma, Cyclic neutropenia, Deficiency of interleukin-1 receptor antagonist, Dendritic cell, monocyte, B lymphocyte, and natural killer lymphocyte deficiency, Dyskeratosis congenital, Dyskeratosis congenita autosomal dominant, Dyskeratosis congenita autosomal recessive, Dyskeratosis congenita X-linked, Epidermodysplasia verruciformis, Familial amyloidosis, Finnish type, Familial cold autoinflammatory syndrome, Familial Mediterranean fever, Familial mixed cryoglobulinemia, Felty's syndrome, Glycogen storage disease type 1B, Griscelli syndrome type 2, Hashimoto encephalopathy, Hashimoto's syndrome, Hemophagocytic lymphohistiocytosis, Hennekam syndrome, Hepatic venoocclusive disease with immunodeficiency, Hereditary folate malabsorption, Hermansky Pudlak syndrome 2, Herpes simplex encephalitis, Hoyeraal Hreidarsson syndrome, Hyper IgE syndrome, Hyper-IgD syndrome, ICF syndrome, Idiopathic acute eosinophilic pneumonia, Idiopathic CD4 positive T-lymphocytopenia, IL12RB1 deficiency, Immune defect due to absence of thymus, Immune dysfunction with T-cell inactivation due to calcium entry defect 1, Immune dysfunction with T-cell inactivation due to calcium entry defect 2, Immunodeficiency with hyper IgM type 1, Immunodeficiency with hyper IgM type 2, Immunodeficiency with hyper IgM type 3, Immunodeficiency with hyper IgM type 4, Immunodeficiency with hyper IgM type 5, Immunodeficiency with thymoma, Immunodeficiency without anhidrotic ectodermal dysplasia, Immunodysregulation, polyendocrinopathy and enteropathy X-linked, Immunoglobulin A deficiency 2, Intestinal atresia multiple, IRAK-4 deficiency, Isolated growth hormone deficiency type 3, Kawasaki disease, Large granular lymphocyte leukemia, Leukocyte adhesion deficiency type 1, LRBA deficiency, Lupus, Lymphocytic hypophysitis, Majeed syndrome, Melkersson-Rosenthal syndrome, MHC class 1 deficiency, Muckle-Wells syndrome, Multifocal fibrosclerosis, Multiple sclerosis, MYD88 deficiency, Neonatal systemic lupus erythematosus, Netherton syndrome, Neutrophil-specific granule deficiency, Nijmegen breakage syndrome, Omenn syndrome, Osteopetrosis autosomal recessive 7, Palindromic rheumatism, Papillon Lefevre syndrome, Partial androgen insensitivity syndrome, PASLI disease, Pearson syndrome, Pediatric multiple sclerosis, Periodic fever, aphthous stomatitis, pharyngitis and adenitis, PGM3-CDG, Poikiloderma with neutropenia, Pruritic urticarial papules plaques of pregnancy, Purine nucleoside phosphorylase deficiency, Pyogenic arthritis, pyoderma gangrenosum and acne, Relapsing polychondritis, Reticular dysgenesis, Sarcoidosis, Say Barber Miller syndrome, Schimke immunoosseous dysplasia, Schnitzler syndrome, Selective IgA deficiency, Selective IgM deficiency, Severe combined immunodeficiency, Severe combined immunodeficiency due to complete RAG1/2 deficiency, Severe combined immunodeficiency with sensitivity to ionizing radiation, Severe combined immunodeficiency, Severe congenital neutropenia autosomal recessive 3, Severe congenital neutropenia X-linked, Shwachman-Diamond syndrome, Singleton-Merten syndrome, SLC35C1-CDG (CDG-IIc), Specific antibody deficiency, Spondyloenchondrodysplasia, Stevens-Johnson syndrome, T-cell immunodeficiency, congenital alopecia and nail dystrophy, TARP syndrome, Trichohepatoenteric syndrome, Tumor necrosis factor receptor-associated periodic syndrome, Twin to twin transfusion syndrome, Vici syndrome, WHIM syndrome, Wiskott Aldrich syndrome, Woods Black Norbury syndrome, X-linked agammaglobulinemia, X-linked lymphoproliferative syndrome, X-linked lymphoproliferative syndrome 1, X-linked lymphoproliferative syndrome 2, X-linked magnesium deficiency with Epstein-Barr virus infection and neoplasia, X-linked severe combined immunodeficiency, and ZAP-70 deficiency.

The disclosed compositions and methods can also be used to treat autoimmune diseases or disorders. Exemplary autoimmune diseases or disorders, which are not mutually exclusive with the immune system disorders described above, include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Bald disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

### 3. Other Disease or Disorders

In some forms the methods administer modified T cells including GUAR protein(s), optionally together with a monoclonal antibody targeting a molecule or receptor on a cell that is associated with, causative of or otherwise likely to cause a disease or disorder in a subject in need thereof. For example, in some forms the methods treat one or more genetic disease or disorders in a subject, such as a hereditary genetic disease or disorder, or a somatic genetic disease or disorder in a subject.

Any of the methods can include treating a subject having an underlying disease or disorder. For example, in some forms, the methods treat a disease or disorder, such as a cancer or auto-immune disease in a patient having another disease or disorder, such as diabetes, a bacterial infection (e.g., Tuberculosis), viral infection (e.g., Hepatitis, HIV, HPV infection, etc.), or a drug-associated disease or disorder. In some forms, the methods treat an immunocompromised subject. In some forms, the methods treat a subject having a disease of the kidney, liver, heart, lung, brain, bladder, reproductive system, bowel/intestines, stomach, bones or skin.

### B. Effective Amounts

In some forms the methods administer modified T cells including GUAR T protein(s), optionally together with a monoclonal antibody targeting a molecule or receptor on a target cell that is to be killed by the GUAR T cell in an amount effective to treat or prevent one or more disease or disorder in the subject.

The effective amount or therapeutically effective amount of a pharmaceutical compositions including modified cells, such as therapeutic GUAR T cells, optionally together with a monoclonal antibody targeting a molecule or receptor on a target cell that can be a dosage sufficient to treat, inhibit, or alleviate one or more symptoms of a disease or disorder, such as a cancer or autoimmune disease, or to otherwise provide a desired pharmacologic and/or physiologic effect, for example, reducing, inhibiting, or reversing one or more of the underlying pathophysiological mechanisms underlying a disease or disorder, such as cancer or autoimmune disease.

In some forms, when administration of the pharmaceutical compositions including modified cells, such as therapeutic T cells, including GUAR protein(s) together with a monoclonal antibody targeting a molecule or receptor on a target cell elicits an anti-cancer response, the amount administered can be expressed as the amount effective to achieve a desired anti-cancer effect in the recipient. For example, in some forms, the amount of the pharmaceutical compositions including modified cells, such as therapeutic GUAR T cells together with a monoclonal antibody targeting a molecule or receptor on a target cell, is effective to inhibit the viability or proliferation of cancer cells in the recipient. In some forms, the amount of the pharmaceutical composition including modified cells, such as therapeutic GUAR T cells, is effective to reduce the tumor burden in the recipient, or reduce the total number of cancer cells, and combinations thereof. In some forms, the amount of GUAR T cells required can vary according to the amount of monoclonal antibody targeting a molecule or receptor on a target cell that is required, and vice-versa. In some forms, the monoclonal antibody targeting a molecule or receptor on a target cell is administered in an amount effective to treat or prevent a disease or disorder in a subject that has previously been administered GUAR T cells, or who has not yet to receive any GUAR T cells. Therefore, in some forms, compositions including GUAR T cells are administered to the subject at the same or different time as a composition including a monoclonal antibody targeting a molecule or receptor on a target cell. For example, in some forms, compositions including GUAR T cells are administered to the subject one or more hours, days, or weeks before a composition including a monoclonal antibody targeting a molecule or receptor on a target cell. In other forms, compositions including GUAR T cells are administered to the subject one or more hours, days, or weeks after a composition including a monoclonal antibody targeting a molecule or receptor on a target cell.

In other forms, the amount of the pharmaceutical compositions including modified cells, such as therapeutic GUAR T cells and a monoclonal antibody targeting a molecule or receptor on a target cell, is effective to reduce one or more symptoms or signs of cancer in a cancer patient, or signs of an autoimmune disease in a patient having an autoimmune disease or disorder. Signs of cancer can include cancer markers, such as PSMA levels in the blood of a patient.

The effective amount of the pharmaceutical compositions including modified cells, such as therapeutic GUAR T cells and a monoclonal antibody targeting a molecule or receptor on a target cell, that is required will vary from subject to subject, depending on the species, age, weight and general condition of the subject, the severity of the disorder being treated, and its mode of administration. Thus, it is not possible to specify an exact amount for every pharmaceutical composition. However, an appropriate amount can be determined by one of ordinary skill in the art using only routine experimentation given the teachings herein. For example, effective dosages and schedules for administering the pharmaceutical compositions including therapeutic GUAR T cells, and compositions of monoclonal antibody targeting a molecule or receptor on a target cell can each be determined empirically, and making such determinations is within the skill in the art. In some forms, the dosage ranges for the administration of the compositions including therapeutic GUAR T cells and/or a monoclonal antibody targeting a molecule or receptor on a target cell are those large enough to effect reduction in cancer cell proliferation or viability, or to reduce tumor burden for example.

The dosage should not be so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the disease to be treated. The dosage can be adjusted by the individual physician in the event of any counter-indications. It will also be appreciated that the effective dosage of the composition including therapeutic GUAR T cells and/or a monoclonal antibody targeting a molecule or receptor on a target cell used for treatment can increase or decrease over the course of a particular treatment. Changes in dosage can result and become apparent from the results of diagnostic assays.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

It can generally be stated that a pharmaceutical composition containing GUAR-T cells described herein can be administered at a dosage of 10⁴ to 10⁹ cells/kg body weight, preferably 10⁵ to 10⁷ cells/kg body weight, including all integer values within those ranges. In some forms, patients can be treated by infusing a disclosed pharmaceutical composition containing GUAR-T expressing cells (e.g., T cells) in the range of about 10⁴ to 10¹² or more cells per square meter of body surface (cells/m).

The infusion can be repeated as often and as many times as the patient can tolerate until the desired response is achieved. GUAR-T cell compositions and/or compositions of a monoclonal antibody targeting a molecule or receptor on a target cell can also be administered once or multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. In some forms, the unit dosage of GUAR T-cells and/or of the monoclonal antibody is in a unit dosage form for intravenous injection. In some forms, the unit dosage is in a unit dosage form for oral administration. In some forms, the unit dosage is in a unit dosage form for inhalation. In some forms, the unit dosage is in a unit dosage form for intra-tumoral injection.

Treatment can be continued for an amount of time sufficient to achieve one or more desired therapeutic goals, for example, a reduction of the amount of cancer cells relative to the start of treatment, or complete absence of cancer cells in the recipient. Treatment can be continued for a desired period of time, and the progression of treatment can be monitored using any means known for monitoring the progression of anti-cancer treatment in a patient. In some forms, administration is carried out every day of treatment, or every week, or every fraction of a week. In some forms, treatment regimens are carried out over the course of up to two, three, four or five days, weeks, or months, or for up to 6 months, or for more than 6 months, for example, up to one year, two years, three years, or up to five years.

The efficacy of administration of a particular dose of the pharmaceutical compositions including modified cells, such as therapeutic T cells, according to the methods described herein can be determined by evaluating the aspects of the medical history, signs, symptoms, and objective laboratory tests that are known to be useful in evaluating the status of a subject in need for the treatment of cancer or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious. In some forms, efficacy is assessed as a measure of the reduction in tumor volume and/or tumor mass at a specific time point (e.g., 1-5 days, weeks, or months) following treatment.

### C. Modes of Administration

In some forms the methods administer modified T cells including GUAR-T protein(s) and monoclonal antibody in combination with a pharmaceutically acceptable carrier. The compositions described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, PA, which discloses typical carriers and conventional methods of preparing pharmaceutical compositions that can be used in conjunction with the preparation of formulations of the therapeutics described herein and which is incorporated by reference herein. These most typically would be standard carriers for administration of compositions to humans. In one aspect, for humans and non-humans, these include solutions such as sterile water, saline, and buffered solutions at physiological pH. Other therapeutics can be administered according to standard procedures used by those skilled in the art.

The pharmaceutical compositions including modified cells, such as therapeutic T cells, described herein can include, but are not limited to, carriers, thickeners, diluents, buffers, preservatives, surface active agents and the like in addition to the therapeutic(s) of choice.

Pharmaceutical compositions containing one or more modified cells, such as therapeutic T cells including GUAR-T protein(s), and monoclonal antibody and optionally one or more additional therapeutic agents can be administered to the subject in a number of ways depending on whether local or systemic treatment is desired, and on the area to be treated. Thus, for example, a pharmaceutical composition including modified cells, such as therapeutic GUAR T-cells, can be administered as an intravenous infusion, or directly injected into a specific site, for example, into or surrounding a tumor. Moreover, a pharmaceutical composition can be administered to a subject as an ophthalmic solution and/or ointment to the surface of the eye, vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. In some forms, the compositions are administered directly into a tumor or tissue, e.g., stereotactically.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant including a porous, non-porous, or gelatinous material).

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Administration of the pharmaceutical compositions containing one or more genetically modified cells (e.g., GUAR T cells) and/or a monoclonal antibody can be localized (i.e., to a particular region, physiological system, tissue, organ, or cell type) or systemic.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular forms only and is not intended to be limiting.

### D. Combination Therapy

In some forms the methods administer modified T cells including GUAR protein(s) and/or a monoclonal antibody in combination with other therapeutic agents or treatment modalities. Any of the disclosed pharmaceutical compositions including modified cells, such as therapeutic T cells (e.g., containing a population of GUAR T-cells), can be used alone, or in combination with other therapeutic agents or treatment modalities, for example, chemotherapy or stem-cell transplantation. As used herein, “combination” or “combined” refer to either concomitant, simultaneous, or sequential administration of the therapeutics.

In some forms, the pharmaceutical compositions and other therapeutic agents are administered separately through the same route of administration. In other forms, the pharmaceutical compositions and other therapeutic agents are administered separately through different routes of administration. The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

Examples of preferred additional therapeutic agents include other conventional therapies known in the art for treating the desired disease, disorder or condition. In some forms, the therapeutic agent is one or more other targeted therapies (e.g., a targeted cancer therapy) and/or immune-checkpoint blockage agents (e.g., anti-CTLA-4, anti-PD1, and/or anti-PDL1 agents such as antibodies).

The compositions and methods described herein may be used as a first therapy, second therapy, third therapy, or combination therapy with other types of therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

The disclosed pharmaceutical compositions and/or other therapeutic agents, procedures or modalities can be administered during periods of active disease, or during a period of remission or less active disease. The pharmaceutical compositions can be administered before the additional treatment, concurrently with the treatment, post-treatment, or during remission of the disease or disorder. When administered in combination, the disclosed pharmaceutical compositions and the additional therapeutic agents (e.g., second or third agent), or all, can be administered in an amount or dose that is higher, lower or the same than the amount or dosage of each agent used individually, e.g., as a monotherapy. In certain forms, the administered amount or dosage of the disclosed pharmaceutical composition, the additional therapeutic agent (e.g., second or third agent), or all, is lower (e.g., at least 20%, at least 30%, at least 40%, or at least 50%) than the amount or dosage of each agent used individually, e.g., as a monotherapy (e.g., required to achieve the same therapeutic effect).

### 1. Additional Anti-Cancer Agents

In some forms, the methods administer one or more additional anti-cancer agents to a subject.

In the context of cancer, targeted therapies are therapeutic agents that block the growth and spread of cancer by interfering with specific molecules (“molecular targets”) that are involved in the growth, progression, and spread of cancer. Many different targeted therapies have been approved for use in cancer treatment. These therapies include hormone therapies, signal transduction inhibitors, gene expression modulators, apoptosis inducers, angiogenesis inhibitors, immunotherapies, and toxin delivery molecules. Numerous antineoplastic drugs can be used in combination with the disclosed pharmaceutical compositions. In some forms, the additional therapeutic agent is a chemotherapeutic or antineoplastic drug. The majority of chemotherapeutic drugs can be divided into alkylating agents, antimetabolites, anthracyclines, plant alkaloids, topoisomerase inhibitors, monoclonal antibodies, and other anti-tumor agents.

### 2. Additional Therapeutic Agents Against Autoimmune Diseases

In some forms, the methods also include administering one or more conventional therapies for autoimmune diseases to the subject.

Exemplary therapies for autoimmune diseases include immunosuppressive agents, such as steroids or cytostatic drugs, analgesics, non-steroidal anti-inflammatory drugs, glucocorticoids, immunosuppressive and immunomodulatory agents, such as methotrexate, leflunomide, hydroxychloroquine, and sulfasalazine. In some forms, the methods administer one or more disease-modifying antirheumatic drugs (DMARDs). In some forms, the methods administer one or more biologic agents for localized treatment (i.e., agents that do not affect the entire immune system), such as TNF-α inhibitors, belimumab and rituximab depleting B cells, T-cell co-stimulation blocker, anti-interleukin 6 (IL-6), anti-IL-1, and protein kinase inhibitors. In other forms, the methods also administer one or more monoclonal antibodies (mAbs), such as anti-TNFα, anti-CD19, anti-CD20, anti-CD22, and anti-IL6R, or other mAbs that target multiple B cell subtypes, and other aberrant cells in autoimmune diseases.

### IV. Kits

The compositions, reagents, and other materials for cellular genomic engineering can be packaged together in any suitable combination as a kit useful for performing, or aiding in the performance of, the methods. It is useful if the components in a given kit are designed and adapted for use together in the method. For example, kits with one or more compositions for administration to a subject, may include a pre-measured dosage of the composition in a sterile needle, ampule, tube, container, or other suitable vessel. The kits may include instructions for dosages and dosing regimens.

Provided are kits containing a transposon (e.g., SB transposon), an AAV vector, mRNA encoding a transposase enzyme (e.g., SB100X transposase) or a vector suitable of expressing the mRNA, and instructional material for use thereof. In preferred forms, the kit includes a plurality of vectors, where each vector independently contains a transposon encoding one or more genes for insertion into a host cell genome, such as a GUAR expression cassette. In some forms, the kit contains a population of cells (e.g., T cells) collectively containing the AAV and/or transposon. The instructional material can include a publication, a recording, a diagram, or any other medium of expression which can be used to communicate the usefulness of the compositions and methods of the kit. All of the kits can further include one or more compositions of human monoclonal antibodies (hmAb). Compositions can include human monoclonal antibodies in a solid (i.e., dry powder or lyophilized) form, or as a solution, such as an aqueous solution. In some forms, the kits includes a composition including an hmAb that is specific for an antigen selected from a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof. In some forms the hmAb targets one or more antigens selected from the group including GPRC5D, AFP, AKAP 4, ALK, Androgen receptor, B7H3, BCMA, Bcr Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD2, GD3, GloboH, GM3, gp100, GPC3, HER 2/neu, HER2, HMWMAA, HPV E6/E7, hTERT, Idiotype, IL12, IL13RA2, IM19, IX, LCK, Legumain, IgK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR β, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, and XAGE.

In some forms, the instructional material may provide instructions for methods using the kit components, such as performing transfections, transductions, infections, and conducting screens. In some forms, kits include a transposon and a transposase. In some forms, kits include an Adeno-associated virus (AAV) vector. In some forms, kits include a transposon including a gene of interest having a reporter gene, a GUAR, or combinations thereof. In some forms, kits include a transposon that includes a promoter and/or polyadenylation signal operationally linked to a reporter gene and/or a GUAR; in some forms, the kit includes a transposon including a GUAR and also includes a composition including an hmAb that is specific for an antigen selected from a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.

In some forms, the kit includes mRNA encoding transposase that incorporates N6 methyladenosine (m6A), 5 methylcytosine (m5C), pseudouridine (ψ), N1 methylpseudouridine (me1ψ), 5 methoxyuridine (5moU), a 5′ cap, a poly(A) tail, one or more nuclear localization signals, or combinations thereof; in some forms, the mRNA, or the transposon, or both are codon optimized for expression in a eukaryotic cell.

In exemplary forms, the kits include a viral vector that is AAV6 or AAV9, and/or cells. Exemplary cells include a T cell, hematopoietic stem cell (HSC), macrophage, natural killer cell (NK), or dendritic cell (DC). In some forms, the T cell is a CD8+ T cell selected from effector T cells, memory T cells, central memory T cells, and effector memory T cells. In some forms, the T cell is a CD4+ T cell selected from Th1 cells, Th2 cells, Th17 cells, and Treg cells.

The disclosed compositions and methods can be further understood through the following numbered paragraphs.
1. A chimeric gamma delta T-cell receptor (γδTCR) including constant and variable immunoglobulin domains,
wherein the constant immunoglobulin domains include a gamma TCR constant domain and a delta TCR constant domain,
wherein the variable immunoglobulin domains include an immunoglobulin antigen binding variable heavy domain, and an immunoglobulin antigen binding variable light domain,
wherein the antigen binding domains specifically bind constant domain(s) of human immunoglobulin IgG.
2. The chimeric γδTCR of paragraph 1, including the amino acid sequence of any one or more of SEQ ID NOs:1-2, optionally light and heavy chain variable regions including the CDRs of SEQ ID NOS:3 and/or 4, or SEQ ID NOS:3 and/or 4, or functional variant having at least 75% sequence identity to any one or more of SEQ ID NOs:1-4.
3. The chimeric γδTCR of paragraph 1 or 2, including all of SEQ ID NOs:1-4.
4. The chimeric γδTCR of any one of paragraphs 1-3, further including a removable masking moiety that prevents the antigen binding domains specifically binding to constant domain(s) of human immunoglobulin IgG.
5. The chimeric γδTCR of paragraph 4, wherein the masking moiety includes a coiled-coil peptide selected from the group including CC2B, CC3, CC4, and CC5.
6. The chimeric γδTCR of paragraph 4 or 5, wherein the masking moiety is a protease-cleavable masking moiety that is removed in the presence of a protease enzyme.
7. The chimeric gamma delta TCR receptor of paragraph 6, wherein the protease is a urokinase.
8. The chimeric γδTCR of any one of paragraphs 4 to 7, wherein the masking moiety includes LSGRSDNH (SEQ ID NO:5).
9. The chimeric γδTCR of any one of paragraphs 1-8, further including one or more intracellular domain(s) of a costimulatory molecule selected from CD27, CD28, CD137, 0X40, IL2Rβ, ICOS, IL7Rα, CD30, CD40, CD3, LFA 1, ICOS, CD2, CD7, LIGHT, NKG2C, B7 H3, ligands of CD83.
10. The chimeric γδTCR of any one of paragraphs 1-9, including the intracellular domain of CD28 associated with the delta TCR constant domain.
11. The chimeric γδTCR of any one of paragraphs 1-10, including the intracellular domain of any one of 0X40, IL2Rβ, ICOS, or IL7Rα, associated with the gamma TCR constant domain.
12. The chimeric γδTCR of any one of paragraphs 9-11, including SEQ ID NOs:83-96.
13. A nucleic acid encoding or expressing the chimeric γδTCR of any one of paragraphs 1-12.
14. A vector including the nucleic acid of paragraph 13.
15. A cell including the chimeric γδTCR of any one of paragraphs 1-12, or the nucleic acid of paragraph 13, or the vector of paragraph 14.
16. A genetically modified T-cell expressing the chimeric γδTCR of any one of paragraphs 1-12 at the cell surface,
wherein the genetically modified T-cell is activated upon binding of the variable antigen binding domains to the constant domain(s) of human immunoglobulin IgG.
17. The cell of any one of paragraphs 15-16 including one or more additional genetic modifications in a gene selected from the group including TRAC, TRBC, B2M and CIITA.
18. A population of cells derived by expanding the cell of any one of paragraphs 15-16.
19. A pharmaceutical composition including the population of cells of paragraph 18 and a pharmaceutically acceptable buffer, carrier, diluent or excipient.
20. The pharmaceutical composition of paragraph 19, further including one or more clones of human monoclonal antibodies.
21. The pharmaceutical composition of paragraph 20, wherein the one or more clones of human monoclonal antibodies includes antibodies that specifically bind to an antigen expressed on a cancer cell.
22. The pharmaceutical composition of paragraph 20, wherein the human monoclonal antibodies specifically bind to anti-GPRC5D antibody.
23. A method of treating a subject having a disease, disorder, or condition including administering to the subject an effective amount of the pharmaceutical composition of any one of paragraphs 18-22.
24. A method of treating a subject having a disease, disorder, or condition associated with an elevated expression or specific expression of an antigen, the method including administering to the subject an effective amount of a pharmaceutical composition including a chimeric γδTCR of any one of paragraphs 1-12, and a human monoclonal antibody that targets the antigen.
25. The method of paragraph 24, wherein the monoclonal antibody targets an antigen expressed on a cancer cell.
26. The method of paragraph 24 or 25, wherein the monoclonal antibody is administered to the patient before, or after the pharmaceutical composition including the chimeric γδTCR.
27. The method of paragraph 24 or 25, wherein the monoclonal antibody is administered to the patient at the same time as the pharmaceutical composition including the chimeric γδTCR.
28. The method of any one of paragraphs 23 to 27, wherein the monoclonal antibody is administered to the patient via the same or different route of administration as the pharmaceutical composition including the chimeric γδTCR.
29. The method of any one of paragraphs 23-28, wherein the subject has cancer, or has been identified as being at increased risk of getting cancer.
30. The method of any one of paragraphs 23-29, wherein the population of cells were isolated from, or derived from the expansion of a cell obtained from the subject having the disease, disorder, or condition prior to the introduction to the cell.
31. The method of any one of paragraphs 23-29, wherein the population of cells were isolated from, or derived from the expansion of a cell obtained from a healthy donor.
32. A kit including a cell of any one of paragraphs 15-17, and/or the nucleic acid of paragraph 13, and/or the vector of paragraph 14,
optionally wherein the kit further includes a human monoclonal antibody.
33. An antigen binding protein that specifically binds to GPRC5D, including a light chain variable region including the CDRs of SEQ ID NO:19 (optionally SEQ ID NOS:24-26) optionally the light chain variable region of SEQ ID NO:19 or a variant thereof with at least 70% sequence identity thereto; a heavy chain variable region including the CDRs of SEQ ID NO:17 (optionally SEQ ID NOS:21-23) optionally the heavy chain variable region of SEQ ID NO:17 or a variant thereof with at least 70% sequence identity thereto; or more preferably a combination thereof, or an antigen-binding fragment or variant thereof.
34. An antigen binding protein that specifically binds to human IgG1, comprising has a light chain variable region including the CDRs of SEQ ID NO:3 (optionally SEQ ID NOS:27-29) optionally the light chain variable region of SEQ ID NO:3 or a variant thereof with at least 70% sequence identity thereto; a heavy chain variable region including the CDRs of SEQ ID NO:4 (optionally SEQ ID NOS:30-32) optionally the heavy chain variable region of SEQ ID NO:4 or a variant thereof with at least 70% sequence identity thereto; or more preferably a combination thereof, or an antigen-binding fragment or variant thereof.
35. The antigen binding protein of paragraphs 33 or 34, wherein the antigen binding protein is an intact monoclonal antibody.
36. The antigen binding protein of paragraphs 33 or 34, wherein the antigen binding protein is a Fab, F(ab′)2, Fab′, Fv, recombinant IgG (rlgG) fragment, single chain antibody, optionally a single chain variable fragment or fusion (scFv), a single domain antibody optionally a sdAb, sdFv, or nanobody.
37. The antigen binding protein of any one of paragraphs 33-36, wherein the antigen binding protein is an intrabody, peptibody, chimeric antibody, fully human antibody, humanized antibody, a heteroconjugate antibody, a multispecific antibody optionally a bispecific, antibody, diabody, triabody, and tetrabody, tandem di-scFv, tandem tri scFv.
38. The antigen binding protein of any one of paragraphs 33-37 with a therapeutic or diagnostic compounds conjugated thereto, optionally wherein the therapeutic or diagnostic compound is a chemotherapeutic agent, radioisotope, or a fluorophore.
39. A polypeptide comprising the amino acid sequence of SEQ ID NO:6 or a variant thereof with at least 70% sequence identity thereto, optionally comprising the CDRs of SEQ ID NOS:27-29.
40. A polypeptide comprising the amino acid sequence of SEQ ID NO:7 or a variant thereof with at least 70% sequence identity thereto, optionally comprising the CDRs of SEQ ID NOS:30-32.
41. A chimeric gamma delta T-cell receptor (γδTCR) comprising the polypeptides of paragraphs 39 and/or 40.

### EXAMPLES

### Example 1: Development of a Potent and Fully Human hIgG1-Fc Binder Via Humanized Mouse Vaccination and Single B Cell Sequencing

### Material and Methods

### Expression and Purification of Recombinant Proteins

The coding sequence of fragment crystalline (Fc) region of human IgG1 isotype (Glu99-Lys330) (one aa mutation, 103 Cys/Ser) was amplified from pFUSEss-CHIg-hG1 (InvivoGen, pfusess-hchg1) (L. Peng et al., Nat Commun 13, 1638 (2022)) and subcloned into a pFuse expression vector with 8×His tag on N-terminus. The recombinant proteins (hIgG1-Fc) were expressed through transfection of hIgG1-Fc expression plasmid into Expi293F cells followed by cell culture for 5 days. The secreted proteins in the medium were purified by affinity chromatography using rProtein A Sepharose Fast Flow beads according to the manufacturer's protocol (Cytiva) and buffer exchanged by Amicon Ultra-4 Centrifugal Filter (MilliporeSigma). Purified hIgG1-Fc proteins were examined by running SDS-PAGE and kept in −80° C. with PBS for further usage.

### Humanized Mice Vaccination

Humanized mice with human IgG and IgK transgene knock-ins (ATX-GK, Alloy Therapeutics) were used for protein-based vaccination, according to a standard (28 days) vaccination schedule. Two humanized mice were subcutaneously injected into the back with 20 μg adjuvant-emulsified hIgG1-Fc proteins on day 0 (prime, CFA) and day 7 (boost, IFA), day 14 (booster, IFA), day 21 (booster, IFA). Retro-orbital blood was collected before initiation of vaccination on day −4, day 13, and day 20. Three days before the termination, an inoculation of 20 μg of hIgG1-Fc proteins in the absence of adjuvant was administered by intraperitoneal injection and anti-sera titers of each humanized mouse were assessed by ELISA before termination.

MACS Enrichment of pan-B Cells from Tissue Samples

Spleen, lymph nodes, and bone marrow were collected from euthanized humanized mice, and single-cell suspensions were prepared as described previously (P. Ren et al., Cell Chem Biol, (2023)) and pooled. Thereafter, pan-B cells (CD19⁺, CD19⁺CD138⁺, CD138⁺) were isolated from freshly prepared single-cell suspensions by immunomagnetic negative selection according to the manufacturer's protocol (STEMCELL, EasySep Mouse Pan-B Cell Isolation Kit, #19844). Non-B cells were labeled with a biotin-antibody cocktail combined with streptavidin-coated magnetic particles and isolated using an EasySep magnet. Enriched pan-B cells were eluted and resuspended in MACS buffer (PBS containing 0.5% (v/v) BSA and 2 mM EDTA). Enriched pan-B cells were counted by using 0.4% (w/v) trypan blue stain and cytoSMART cell counter according to the manufacturer's protocol.

### Isolation of Antigen-Specific Single B Cells by FACS

Enriched pan-B cells were incubated with 20 nM of biotinylated hIgG1-Fc proteins and FcR Blocking Reagent (100-fold dilution, Miltenyi Biotec, #130-059-901) for 30 min on ice. Cells were washed twice and further stained with fluorophore-labeled antibody cocktail (Lei et al., Front Microbiol 10, 672 (2019)) in MACS buffer containing LIVE/DEAD Fixable Near-IR Dead Cell Stain Kit (1000-fold dilution, Invitrogen, L34976), goat anti-mouse FITC-IgM (100-fold dilution, Jackson immune research, #115-096-075), goat anti-mouse Alexa Fluor 594-IgG (100-fold dilution, Jackson immune research, #115-586-071) and APC-streptavidin (Biolegend, #405207) and PE-streptavidin (Invitrogen, #S21388), respectively, for additional 30 min on ice. The stained cells were washed twice and resuspended in pre-chilled MACS buffer and passed through a 70 μm cell strainer (BD Biosciences) before cell sorting. Antigen-specific single B cells were identified and sorted by a FACS Aria III cell sorter (BD Biosciences) at single-cell density into a 15 ml falcon tube containing 4 ml of MACS buffer.

### Single-Cell V(D)J Sequencing and Data Analysis

After FACS isolation, 10,000 of IgG⁺ antigen-specific B cells were loaded on a Chromium Next GEM Chip K as described previously. Briefly, single-cell lysis and cDNA first-strand synthesis were performed using Chromium Next GEM Single Cell 5′Kit v2 according to the manufacturer's protocol. The barcoded cDNA was isolated and amplified according to the manufacturer's protocol for BCR repertoire library preparation. The final library was quantified by D1000 ScreenTape assay (Agilent) for the TapeStation system. Sequencing was performed on an Illumina Miseq platform running MiSeq Reagent Kit v3 (150 Cycles), with a 2×91 bp paired-end reading mode. The average sequencing depth aimed for the library was 5,000 raw read pairs per cell.

### Binding Capacity Measurement by Yeast Surface Display

Paired heavy- and light-chains of the selected top enriched clones were subcloned into modified Fab yeast display vectors pESC-Leu2d and then electroporated into yeast strain S. cerevisiae EBY100, respectively. The transformed cells were cultured in the plate using SD-base with a commercially available drop-out mix composed of all essential amino acids except for tryptophan, according to the manufacturer's instruction. The Fab antibody display and analysis by flow cytometry was described previously (Jia et al., Biotechnol Lett 41, 1067-1076 (2019)). Briefly, a single colony of transformed yeast cells was inoculated into liquid synthetic dextrose containing SG dropout medium (SD-CAA) at 30° C. overnight with sharking, then cells were diluted and induced to synthetic galactose containing SG dropout medium (SG/R-CAA) at 20° C. for overnight with sharking for induction of Fab antibody fused with a c-Myc tag on the yeast surface. After induction, cells were washed twice with MACS buffer and probed with anti-c-Myc chicken IgY fraction (Invitrogen, #A21281) and biotinylated hIgG-Fc protein for 1 h at room temperature. Subsequently, the cells were washed twice with MACS buffer and stained with PE-streptavidin and Alexa Fluor 488 goat anti-chicken (Thermo, #PA1-28794) in dark for an additional 40 min at 4° C. Finally, the stained cells were washed twice with MACS buffer and subjected to analysis by an Attune NxT Acoustic Focusing Cytometer (Thermo Fisher).

### Lentiviral Vector Construction, Production, and Transduction

Lentiviral vectors were used for transducing HEK293 cell lines. The plasmid backbone contained an EF1α promoter, a furin-F2A cleavage peptide, and human TCR gamma (γ, UniProtKB-P0CF51)/delta (δ, UniProtKB-B7Z8K6) chain constant region. To construct anti-hIgG1-Fc-TCRs, individual paired variable heavy (VH)/TCRγ constant chains and variable light (VL)/TCRδ constant chains were amplified by PCR using respective oligo pairs and inserted via Gibson assembly into lentiviral transfer vectors. To detect CD3-TCRγδ complexes expression, the Flag-tag sequence (GATTACAAAGACGATGACGATAAG (SEQ ID NO:9)) was added before VH/TCRγ constant chain and the Myc-tag sequence (GAACAGAAACTGATTTCCGAGGAAGATCTG (SEQ ID NO:10)) was added before VL/TCRδ constant chain. Lentivirus was produced by transfecting HEK293FT cells (Thermo Fisher) in six-well plates as described previously (P. Ma et al., Adv Sci (Weinh) 8, 2003091 (2021)). Briefly, lentiviral transgene plasmids, packing plasmids psPAX2, and envelope plasmids pMD.2G together with Lipofectamine 3000 transfection reagents (Thermo, #L3000015) were cotransfected in pre-seeded HEK293FT cells for lentivirus production. 72 h post-transfection, the virus-containing culture medium supernatant was collected and centrifuged at 4,000 g for 10 min at 4° C. to remove the cell debris. Followed by the virial supernatant was directly used for spinfection, and the transduced cells were isolated by flow sorting or drug selected before further usage.

### AAV Vector Construction, Production, and Titration

To express the anti-hIgG1-Fc-TCR construct at the TRAC locus, the V_{L}-Cδ-F2A-V_{H}-Cγ sequences were amplified from the above lentiviral transgene plasmids and subcloned into AAV crRNA expression vectors containing inverted terminal repeats, which allowing V_{L}-Cδ and V_{H}-Cγ expression under the control of TCRα promoter. Recombinant AAV viruses were produced by transfecting HEK293T cells in 15-cm tissue culture dishes (Corning) as described previously (Dai et al., Nat Methods 16, 247-254 (2019)). Briefly, AAV transgene plasmids, packing plasmids pDF6, and AAV9 serotype plasmids together with polyethyleneimine were mixed and drop-wase added to HEK293T cells for AAV production. 72 h post-transfection, transfected cells were collected, and AAV was purified from the cell lysate using chloroform extraction and concentrated using Amicon Ultra 100 kD MWCO ultrafiltration centrifugal units (Millipore). Viral titer was measured by qPCR using custom Taqman assays (Thermo) targeted to promoter EFS.

### Cleavage of Masked GUAR-J by Recombinant Proteases

A recombinant human urokinase plasminogen activator (uPA) (#10815-H08H-A) was purchased from Sino Biological. The cleavage assay was conducted with activated enzyme as described previously (Mansurov et al., Nat Biomed Eng 6, 819-829 (2022)). Briefly, 0.5 million masked GUAR-J/T cells were incubated and titrated with different concentrations of activated uPA at 37° C. overnight. Subsequently, the cells were washed twice with MACS buffer and used for further staining experiments.

### Animals

All animal work was performed under the guidelines of Yale University Institutional Animal Care and Use Committee (IACUC) with approved protocols. The general health of the mice is in good condition before the cancer-related experiments started. Mice of both male and female under the age of 8-12 weeks were used for experiments. NOD-scid IL2Rgamanull (NSG) mice were purchased from Jackson Laboratory (JAX) and bred in-house for in vivo model and T cell based therapeutic efficacy testing experiments.

### Cell Culture

HEK293T, Jurkat, MM.1R, SKOV3 cell lines were obtained from commercial source (American Type Culture Collection (ATCC)). HEK293T were cultured in DMEM (Gibco) media supplemented with 10% FBS (Sigma) and 200 U/mL penicillin-streptomycin (Gibco), hereafter referred to as D10. Jurkat and MM.1R cells were cultured in RPMI-1640 (Gibco) media supplemented with 10% FBS and 200 U/mL penicillin-streptomycin. MM.1R-PL-BCMA OE cell line was established according to the previous research (L. Ye et al., Cell Metab 34, 595-614 e514 (2022)). SKOV3-GL were established by infecting SKOV3 cells with lentivirus expressing GFP-Luciferase (GL). Infected SKOV3-GL cells were purified by flow sorting of GFP positive cells.

To establish Jurkat-TCR-KO cells, Jurkat cells were electroporated with Cas9 RNP targeting human TRAC and TRBC loci using Neon transfection system according to the manual. Briefly, to prepare Cas9 RNP targeting TRAC and TRBC, TRAC or TRBC crRNA were mixed with tracrRNA respectively at 1:1 (v/v) and annealed by 95° C. for 5 min following 37° C. for 10 min. RNPs were formed by the addition of HiFi SpCas9 nuclease (IDT, 1081061) with 80 μM gRNA to attain a molar ratio of sgRNA-Cas9 of 2:1. Final RNP mixtures were incubated at 37° C. for 15-30 min after a thorough mix. Based on a Cas9 protein basis, 10 pmol or 100 pmol of RNP were used for each electroporation. To do electroporation, 0.2 million cells per 10 μl tip reaction or 2 million cells per 100 μl tip reaction were resuspended in electroporation buffer R. 10 pmol or 100 pmol of RNP (based on a Cas9 protein basis) were mixed with T cells and electroporated using 1350 V, 10 ms, and 3 pulses. After electroporation, the cells were transferred into prewarmed cultural medium immediately. Electroporated Jurkat cells were cultured overnight, and then fresh medium were added to the plate.

### T Cell Isolation, Activation, and Culture

Human peripheral blood mononuclear cells from healthy donors were obtained from commercial source (STEMCELL Technologies, 70025). Frozen peripheral blood mononuclear cells were thawed and cultured overnight in X-vivo 15 medium (Lonza) supplemented with 10% fetal bovine serum with 10 ng/ml hIL-2 and then used for CD3⁺ T cell isolation. T lymphocytes were purified using the Pan T cell isolation kit (Miltenyi Biotech). Then the T cells were activated with Dynabeads (1:1 beads:cells) Human T-Activator CD3/CD28 (ThermoFisher) in T cell culture medium for two days. For T cell culture, the medium was changed every 2 days and cells were replated at 1-1.5 million cells/ml.

### Human γδ T Cell Expansion and Enrichment

Human peripheral blood mononuclear cells from healthy donors were obtained from a commercial source (STEMCELL Technologies, 70025). Frozen peripheral blood mononuclear cells were thawed and cultured within X-vivo 15 medium (Lonza), which supplemented with 10% fetal bovine serum (Corning), 10 ng/ml hIL-2 (Peprotech), and 2.5 μM zoledronic acid (TCI). The culture medium was changed every 2 days and cells were replated at 1-1.5 million/ml. Following expansion, γδ T cells were isolated from PBMCs by flow cytometry using PE anti-human TCR γ/δ Antibody (sorted CD3+TCRγδ+TCRαβ-population).

### RNP Formulation

RNPs were produced by complexing a two-component gRNA to Cas9 protein. The two-component gRNA included a crRNA and a tracrRNA, both chemically synthesized and lyophilized (IDT). Lyophilized RNA was resuspended in nuclease-free duplex buffer (IDT) at a concentration of 160 μM and stored in aliquots at −80° C. To make gRNA, aliquots of crRNA and tracrRNA were thawed, mixed 1:1 v/v and annealed by 95° C. for 5 min following 37° C. for 10 min. RNPs were formed by the addition of HiFi SpCas9 nuclease (IDT, 1081061) with 80 μM gRNA to attain a molar ratio of sgRNA-Cas9 of 2:1. Final RNP mixtures were incubated at 37° C. for 15-30 min after a thorough mix. Based on a Cas9 protein basis, 10 pmol or 100 pmol of RNP were used for each electroporation.

### T Cell Electroporation

After T cell activation, the dynabeads were magnetically removed and the T cells were transfected by electrotransfer of Cas9 RNP using Neon transfection system according to the manual. Briefly, 0.2 million cells per 10 μl tip reaction or 2 million cells per 100 μl tip reaction were resuspended in electroporation buffer R. 10 pmol or 100 pmol of RNP (based on a Cas9 protein basis) were mixed with T cells and electroporated using 1600 V, 10 ms, and 3 pulses. After electroporation, the cells were transferred into prewarmed cultural medium immediately. If the experiment is for gene knockin purpose, AAV were added to the medium supplemented with 1 μM HDR Enhancer (IDT). T cells were cultured overnight, and then fresh medium were added to the plate.

### Antigen-Binding Assessment

Transduced T cells were incubated with soluble biotinylated human IgG1-Fc at indicated concentration on ice for 30 min. After three washes with DPBS, cells were stained with Brilliant Violet 421™ antibody that binds DYKDDDDK (SEQ ID NO:82) Tag (Biolegend, 637321) and PE streptavidin (Biolegend, 405203) on ice for 15 min. Cells were washed for once and then subject to flow cytometry analysis.

### Intracellular Staining for Flow Cytometry

Intracellular staining was performed to detect the expression of cytokines on T cells. Purified GUAR-T cells were co-cultured with cancer cells at E:T ratio of 1:1 for 6 h with or without antibody. For IFNγ, TNFα and IL-2 staining, 5 μg/ml Brefeldin A (Biolegend, 420601) was added during the co-culture. Then, T cells were collected and stained for live/dead fixable near-IR dead cell staining kit (Thermofisher, L34976) and surface markers including FITC anti-human CD4 (Biolegend, 357406) and BV421 anti-human CD8 (Biolegend, 344747) on ice for 15 min. After membrane protein staining, T cells were fixed and permeabilized using BD Cytofix/Cytoperm Fixation/Permeabilization Solution kit (BD, 554714) as per the recommendation of the manufacture, followed by staining with anti-cytokine antibodies including APC anti-human IFNγ (Biolegend, 506510), PE anti-human TNF-α (Biolegend, 502909), and PE anti-human IL-2 (Biolegend, 500306). Flow cytometry was performed on BD FACSAria II and analyzed with FlowJo software.

### Intracellular Phosphoprotein Analyses

Purified GUAR-T cells were serum starved overnight. Then, cells were stained with live/dead fixable near-IR dead cell staining kit (Thermofisher, L34976) and surface markers including FITC anti-human CD4 (Biolegend, 357406) and BV421 anti-human CD8 (Biolegend, 344747) on ice for 15 min. After cell washing, GUAR-T cells were incubated with 3 μg/ml soluble biotinylated human IgG1-Fc on ice for 30 min, washed three times with DPBS, and cross-linked with streptavidin (Biolegend, 405150) at 10 μg/ml for indicated periods of time. After cross-linking, cells were immediately fixed with Phosflow Fix Buffer I (BD Biosciences, 557870) at RT for 10 min. Fixed cells were permeabilized with cold Phosflow Perm Buffer III (BD Biosciences, 558050) for 30 min on ice. Permeabilized samples were stained with antibodies detecting phosphorylated CD3ζ ITAM3 (pY142) (BD Biosciences, 558050), ZAP70 (pY319) (BD Biosciences, 557881) and ERK1/2 (pT202/pY204) (Biolegend, 369505). Flow cytometry was performed on BD FACSAria II and analyzed with FlowJo software.

### CD107a Degranulation Assay

Purified GUAR-T cells were resuspended to a final concentration of 1 million per ml with cultural medium supplemented with 2 μM Monesin (Biolegend, 420701), anti-CD107A PE antibody (Biolegend, 328607), with or without the indicated antibody, and the indicated cancer cells at E:T ratio of 1:1. Cells were co-incubated at 37° C. for 6 hours. Then, T cells were collected and stained for live/dead fixable near-IR dead cell staining kit (Thermofisher, L34976) and surface markers including FITC anti-human CD4 (Biolegend, 357406) and BV421 anti-human CD8 (Biolegend, 344747) on ice for 15 min. Flow cytometry was performed on BD FACSAria II and analyzed with FlowJo software.

### In Vitro Killing Assay

The cytotoxicity of GUAR-T cells was determined by luciferase-based assay. Luciferase expressing cancer cells served as target cells. The effector (E) and tumor target (T) cells were co-cultured in triplicates at the indicated E:T ratio and antibody using white-walled 96-well plates with 5×10⁴ in T cell medium. At the indicated time point after co-culture, 150 μg/ml D-luciferin (PerkinElmer) was directly added to each well. After incubation for 5 min, luciferase assay intensity was measured by plate reader (PerkinElmer).

### Mouse Systemic Tumor Model

To carry out the intraperitoneal xenograft models, NSG mice were initially injected with 3 million luciferase-expressing SKOV3 intraperitoneally. After 3 weeks, 10 million GUAR-T were infused intraperitoneally along with anti-HER2 antibody and human IL-2 if indicated. 3 mg/kg anti-HER2 antibody per mouse was intraperitoneally injected every day for two weeks along with human IL-2 at 2000 IU for each mouse. Tumor burden was measured by IVIS Spectrum (PerkinElmer) and was quantified as total flux (photons per sec) in the region of interest. Images were acquired within 10 min following intraperitoneal injection of 150 mg/kg of D-Luciferin (PerkinElmer). Animal survival was recorded as mice reached their endpoints.

### In Vivo Cytokine Secretion Assay

To determine in vivo IFNγ release level, orbital blood was collected from each mouse after 24 h or 48 h of initial injection of GUAR-T cells along with antibody if indicated. Blood plasma was harvested by centrifuging collected blood for 10 min at 3,000 g. In vivo IFNγ release was measured by ELISA MAX Deluxe Set Human IFN-γ (Biolegend, 430104) according to the manual.

Complex Structure Prediction with AF2-Multimer

The complex structure of GUAT-TCR was generated with AF2-multimer, running locally via Singularity (https://github.com/deepmind/alphafold) on the Yale high-performance computing (HPC) Farnam clusters. Sequences of protein chains were placed in a single FASTA file and used as input for complex modeling prediction. AF2-multimer (version 2.2.4 and 2.3.0) was used by querying corresponding full databases to predict the complex conformations, with default parameters and random-seed settings as 100,000. AF2-multimer generated 25 total predictions using five models (5 predictions per model), ranked by the sum of two predicted structure accuracy measures: ipTM (interface predicted template modeling score) and pTM (predicted template modeling score). Amber relaxations were applied to AF2-multimer predicted structures. The protein structures were analyzed and plotted with PyMol. A later version AlphaFold-2.3.0 (released 2022 Dec. 13) was re-trained with an updated database using identical model architecture, which was expected to generate more accuracy on large protein complexes. Results were output from AlphaFold2 version 2.3.0 unless otherwise specified.

### Results

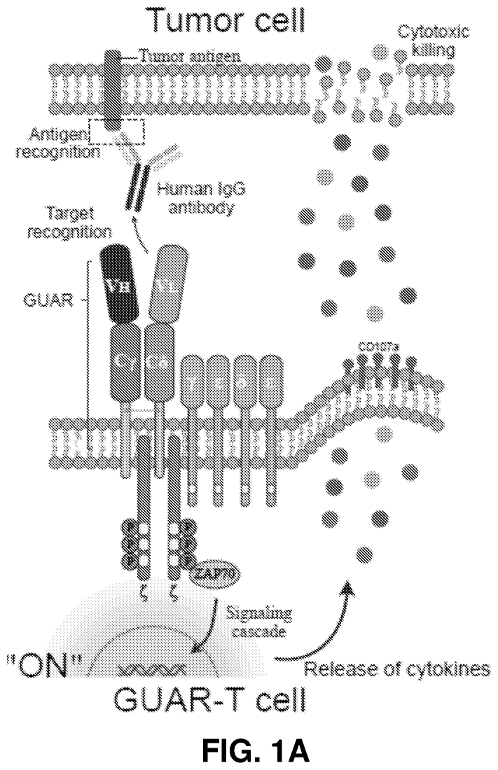

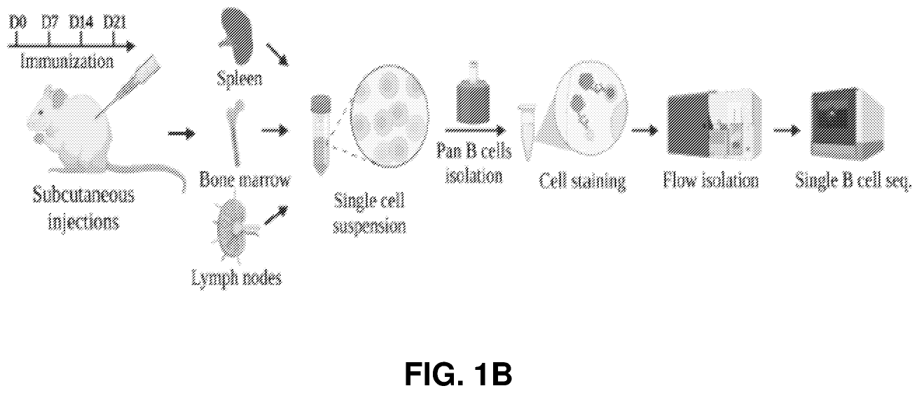

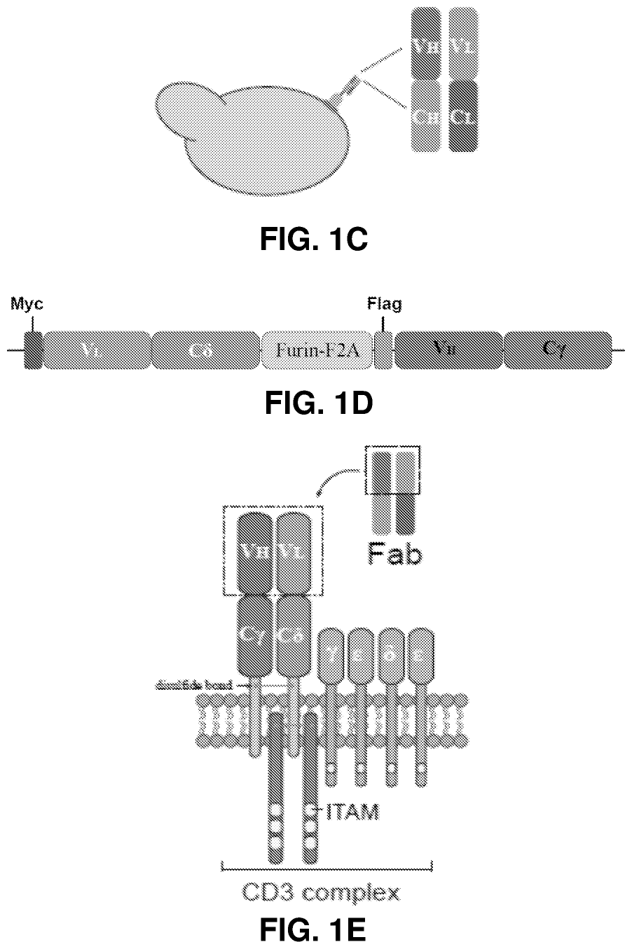

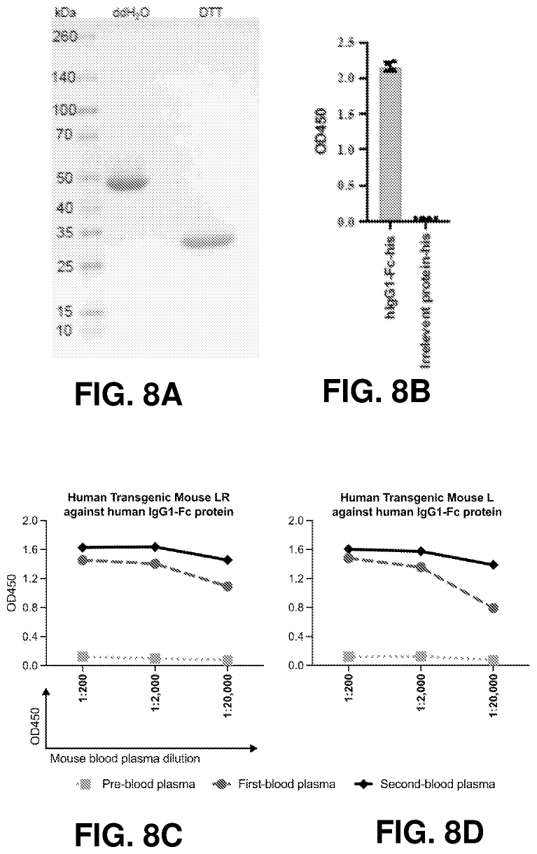

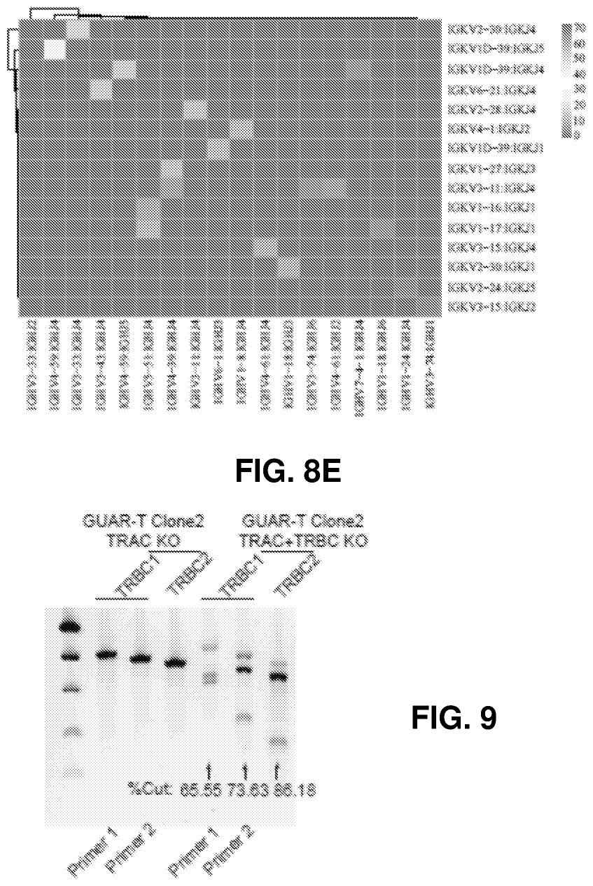

To develop a universal antibody receptor that allows target-specific monoclonal antibody (mAb) adaptation, a potent and fully human hIgG1-Fc binder was first generated using humanized mouse vaccination and single B cell sequencing (FIG. 1B). A customized dimeric version of human IgG1-Fc (hIgG1-Fc) protein that was tagged with an N-terminal polyhistidine (His8) was produced by Expi293F mammalian cells and its purify and binding activity verified using Coomassie-stained SDS-PAGE and ELISA, respectively, thereby confirming that it was assembly and functionally displayed (FIGS. 8A-8B). Standard 28-day mice vaccination procedures were followed, during which the blood samples were collected from each immunized immunoglobulin (Ig) humanized mouse before and after the booster. Each blood sample was labeled as pre-, first, or second vaccination draw according to the collection sequence (FIGS. 8C-8D). Antibody serum binding titers were determined using serial serum dilution on ELISA plates coated with recombinant hIgG1-Fc proteins, and binding activity was visualized using anti-mouse IgG antibodies at 450 nm optical density (OD). Three sequential serum samples exhibited increasing antibody responses during each blood collection (FIGS. 8C-8D). All post-immunized serum samples showed significant reactivity to the recombinant hIgG1-Fc proteins. To further enrich target-binding B cells, spleens, lymph nodes, and bone marrow were collected from both immunized humanized mice and pooled all monocytes together. Isolated pan-B cells (CD19+, CD19+CD138+, and CD138+) by indicated kit (Methods), and then biotinylated hIgG1-Fc protein was utilized to select for target-binding B cells by FACS sorting (data not shown). Thereafter, 10,000 post-sorted target-binding B cells were subjected for single-cell BCR library preparation and sequencing. Subsequently, a total of 974 hIgG1-Fc-binding B cells were identified and 639 paired antibody sequences were detected. These single B-seq data allowed the mapping out of the overall heavy- and light-chain V/J segment recombination patterns in total sequenced B cells, and the landscape of the BCR population by analyzing the paired BCR repertoires (FIG. 8E).

To evaluate the binding properties of top-ranked paired human IgG clones, the top 3 paired heavy- and light-variable segments were cloned into yeast-Fab display vectors, respectively. Thereafter, the plasmids were electroporated into yeast competent cells and over-expressed Fab on the yeast surfaces under galactose induction. As a result, all three top-ranked paired human IgG clones (named Clone-1, Clone-2, and Clone-3) showed positive hIgG1-Fc binding (FIG. 1C). In addition, to further characterize the cross-reactivity of those three clones with other human IgG allotypes, 50 nM of each biotinylated human IgG-Fc protein was incubated with three Fab-displayed yeast cells, respectively. Flow cytometry analysis showed that Fab of Clone-2 can bind all four human IgG allotypes, but no clear hIgG2-Fc binding was detected in Fab of Clone-1 and Clone-3 (data not shown). Therefore, Clone-2 is chosen as a lead clone as a potent and fully human hIgG1-Fc binder for the construction of GUAR.

### Example 2: Reconstitution and Functional Assembly of GUAR in Jurkat and Primary Human T Cells

### Results

According to the monovalent interaction between Fab clones and hIgG-Fc domains, GUAR constructs were further designed that utilize TCR γ- and δ-constant regions to replace Fab heavy- and light-constant regions (FIG. 1D). Furthermore, GUAR-VHCδ and VLCγ chains linked by a furin-F2A cleavable peptide (54), and a Myc tag or a Flag tag was fused at N terminus of each GUAR chain, which facilitates detection of surface expression of GUAR-antibody receptor domains, separately (FIG. 1D). As a proof of concept, GUAR-receptor candidates were individually introduced into Jurkat TCR-αβ-KO cells via lentivirus-mediated infection. GUAR-receptor chains, GUAR-CD3 complex assembly, and functional staining were all confirmed by flow cytometry (FIG. 1E). Thus, the introduction of the GUAR-constructs allowed the generation of modular GUAR-expressing Jurkat cells, termed GUAR-J.

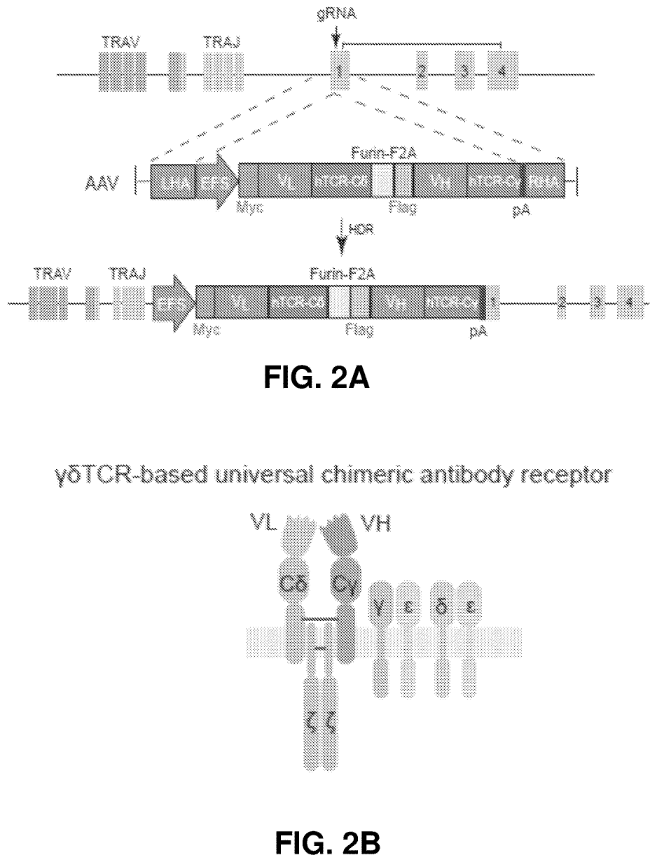

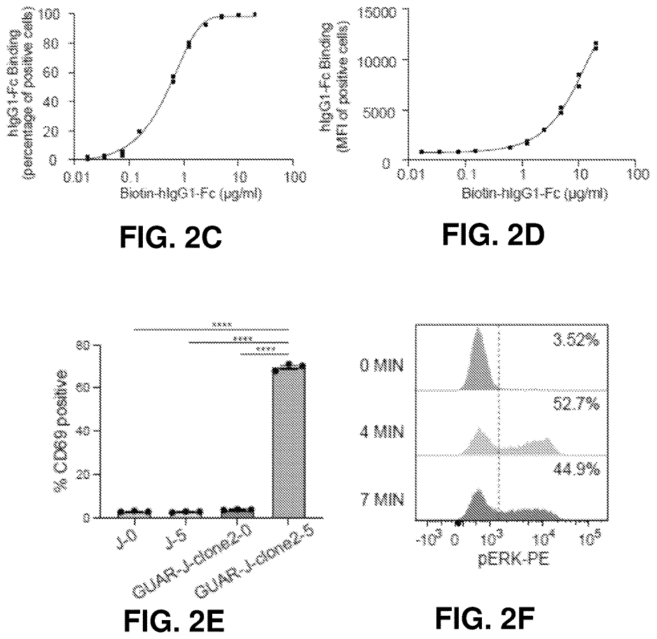

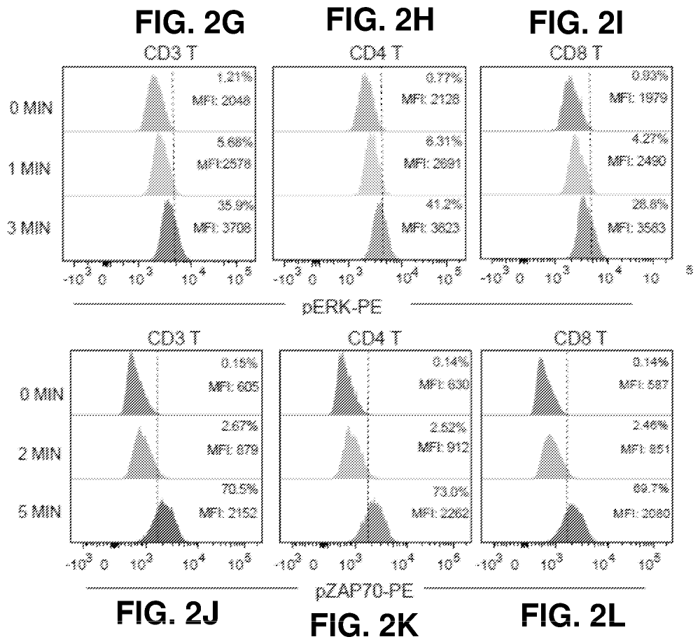

To further validate the performance of GUAR-receptor candidates in human primary T cells, the CD3+ population was isolated from human PBMCs to include all T cells. Using a knock-in/knockout method, the T cells' endogenous αβTCR signaling were ablated via CRISPR ribonucleoprotein (RNP) complex targeting of the TRAC locus, and the signaling of heterodimeric GUAR-γδTCR restored via AAV-mediated transduction and homology-directed repair (HDR) mediated knock-in, while generating GUAR-VHCδ and VLCγ chains linked by a furin-F2A element under the transcriptional control of EFS promoter and polyA elements (FIGS. 2A-2B). Out of the three clones, GUAR-Clone2 had shown robust surface expression of GUAR-CD3 complexes and efficient antigen binding capacity (data not shown). Therefore, GUAR-Clone2 was selected as a binder construct backbone for subsequent GUAR-T experiments. To characterize whether intact TCR β chain interfered with the formation of the GUAR-CD3 complex, TCR β chain-depleted GUAR-T-Clone2 cells were generated to compare with TRAC-GUAR-T-Clone2 cells, and confirm TRBC1 and TRBC2 knockout by surveyor assay (FIG. 9). The flow cytometry results revealed that no significant differences were observed between groups, either in surface expression or antigen-binding capacity, confirming that γδTCR chains do not pair with the endogenous TCRs in αβ T cells, consistent with previous observations. In addition, the antigen binding efficiency of GUAR-T-Clone2 were titrated by surface staining using biotinylated hIgG1-Fc protein under concentration gradients (FIGS. 2C-2D). According to flow cytometry, EC50 was 0.5 μg/mL for human IgG1-Fc, while EC95 was 4.5 μg/mL (FIG. 2C). Median fluorescence intensity (MFI) of antigen binding was enhanced along with the increasing antigen concentration from 0 to 20 μg/mL (FIG. 2D). These data together confirmed the feasibility of reconstitution and functional assembly of GUAR in Jurkat and primary human T cells.

### Example 3: Stably Knock-In GUAR-T Cells Show Potent Antibody-Dependent Functionality In Vitro

### Results

To assess the dynamics of GUAR-Clone2-engineered cell activation, the expression of activation marker CD69 on GUAR-Clone2-transduced Jurkat TCR-αβ KO (GUAR-J-Clone2) cells after co-cultured with immobilized human IgG1-Fc antigen. Upon antigen stimulation, CD69 expression was significantly upregulated in GUAR-J-Clone2 cells compared to non-transduced Jurkat-TCRαβKO cells and rested GUAR-J-Clone2 cells (FIG. 2E), and ERK phosphorylation was dramatically increased at 4 min and 7 min compared to initiation (0 min) under cross-linking measurement, indicating GUAR-Clone2 endowed Jurkat cells have robust antigen response (FIG. 2F). Furthermore, in human primary T cells, enhanced phosphorylation of ERK and ZAP70 were observed in both CD4 and CD8 T cells after biotinylated antigen binding and cross-linking with streptavidin (FIGS. 2G-2L). This data demonstrated that GUAR-Clone2 can mediate functional formation of GUAR-CD3 complexes and activate downstream signaling cascade in T cells upon antigen stimulation.

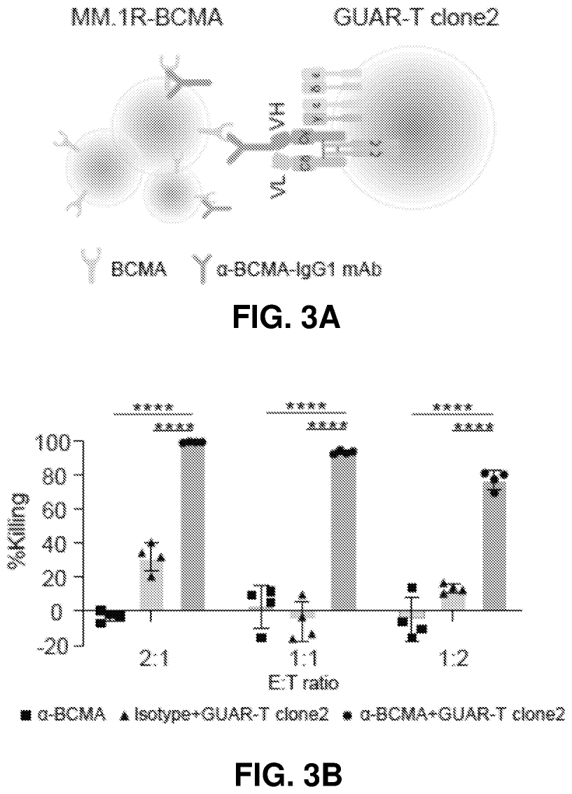

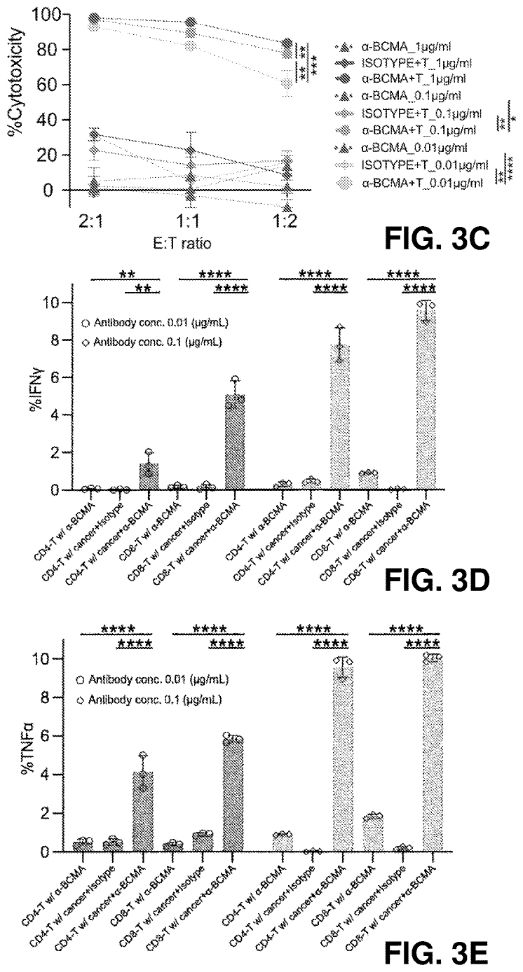

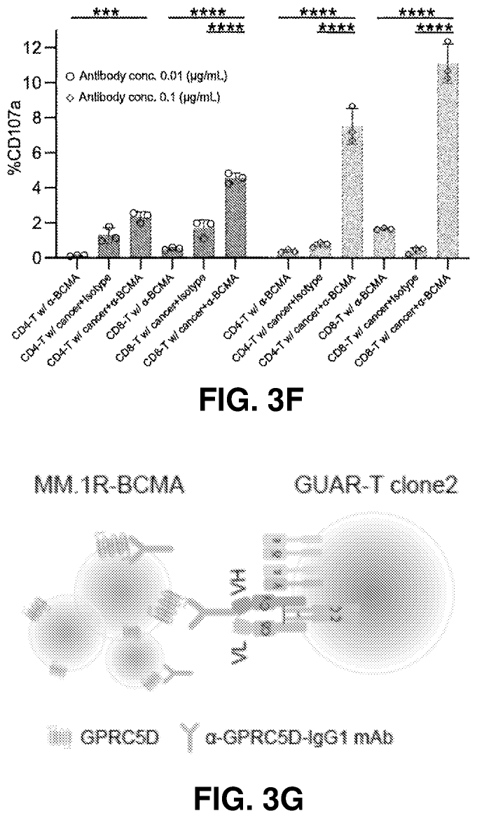

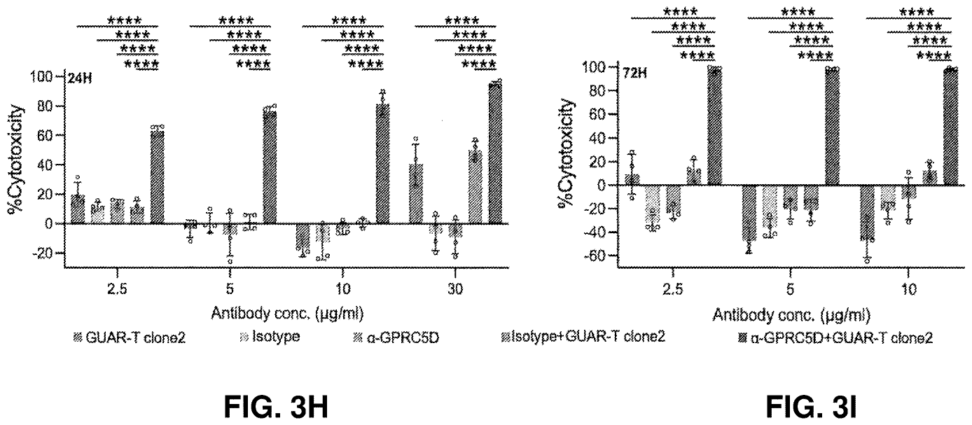

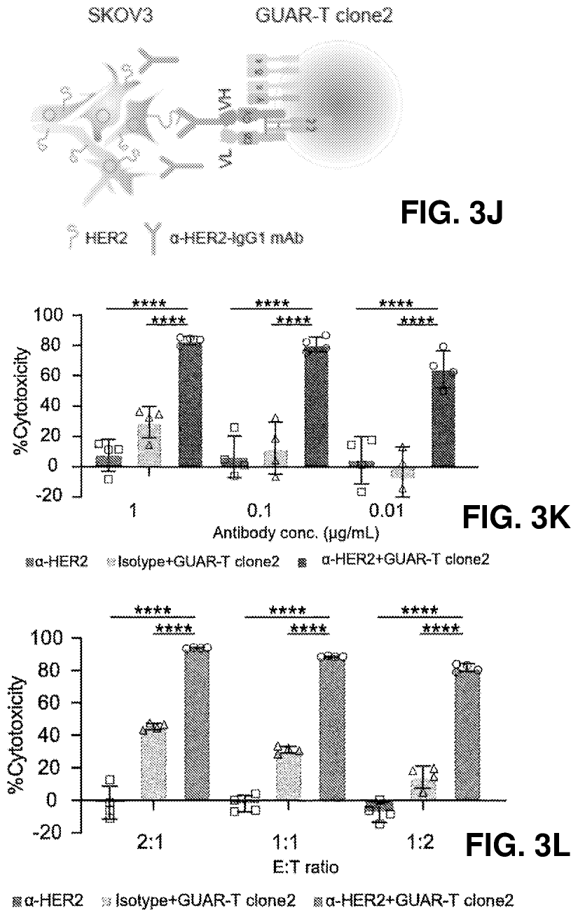

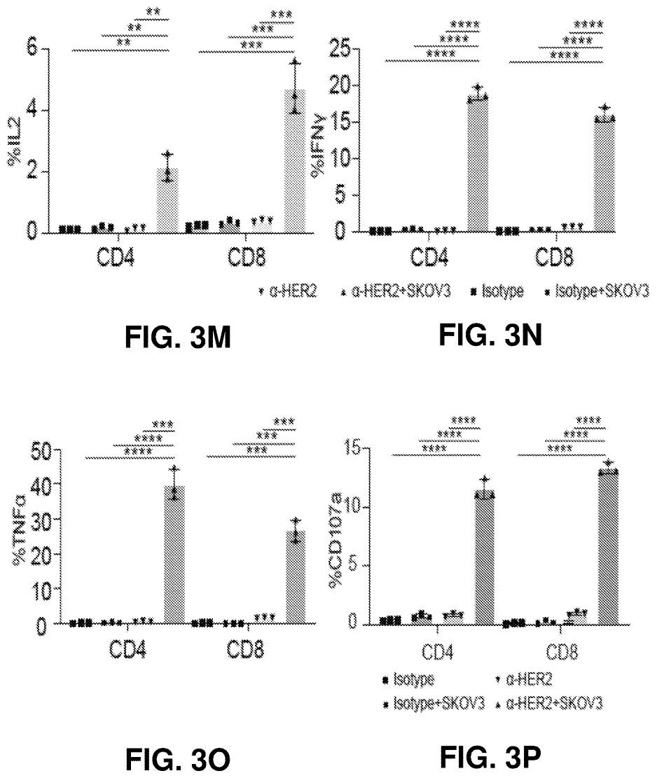

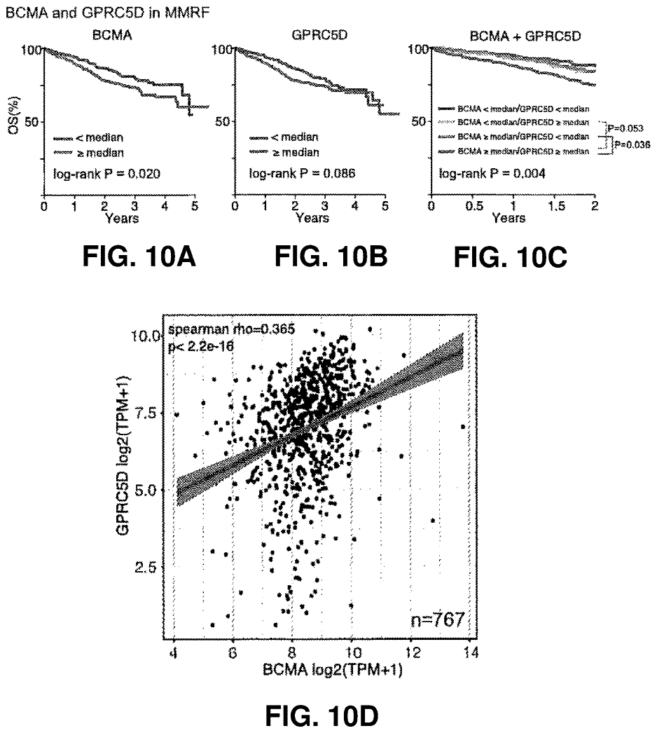

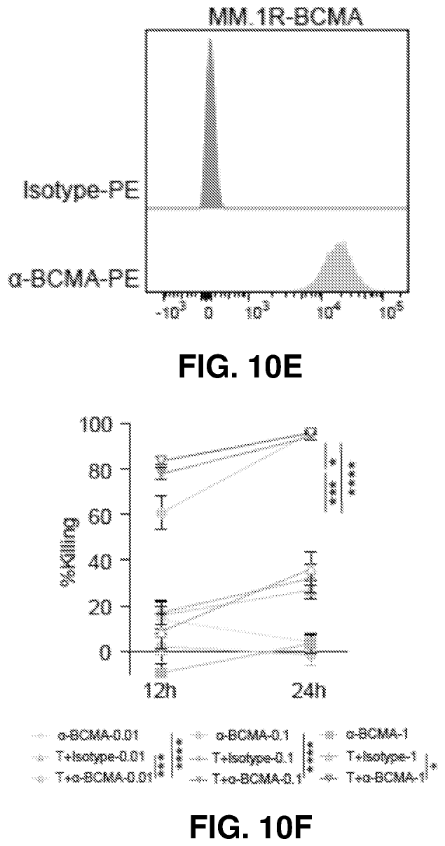

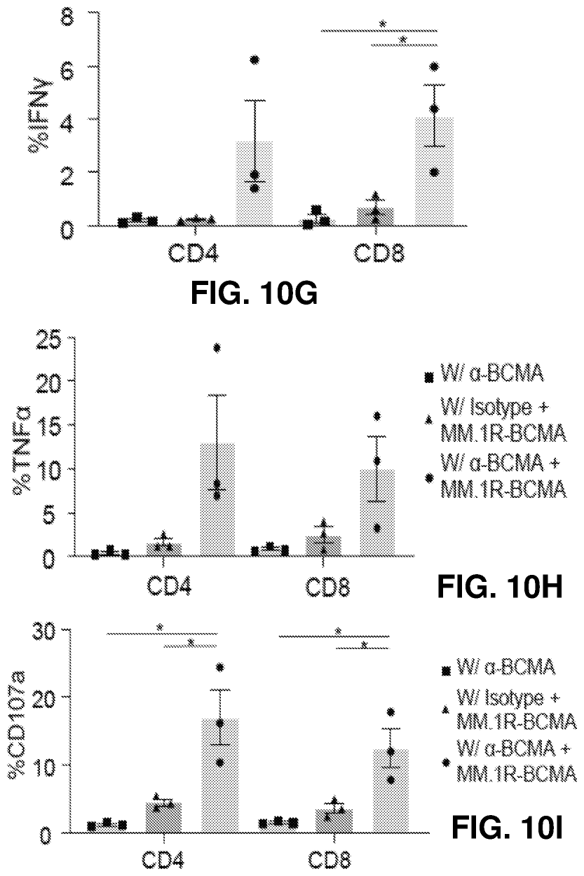

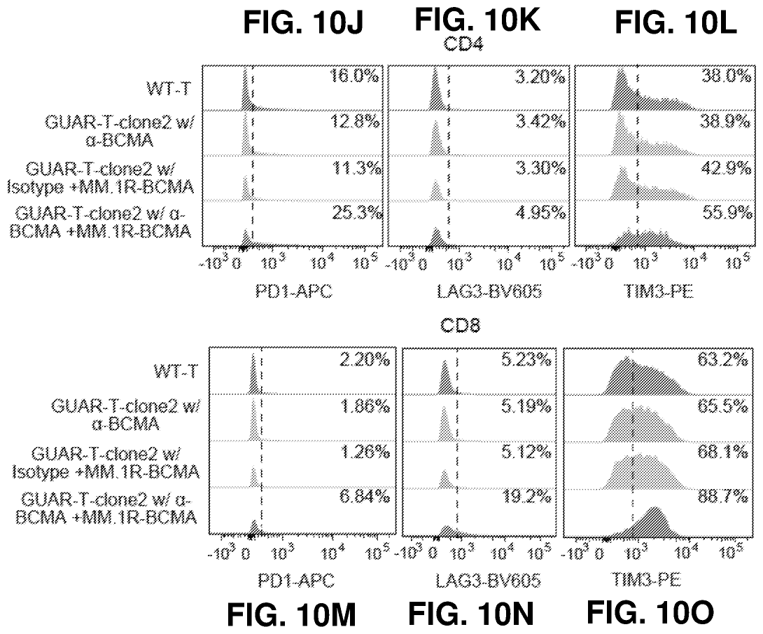

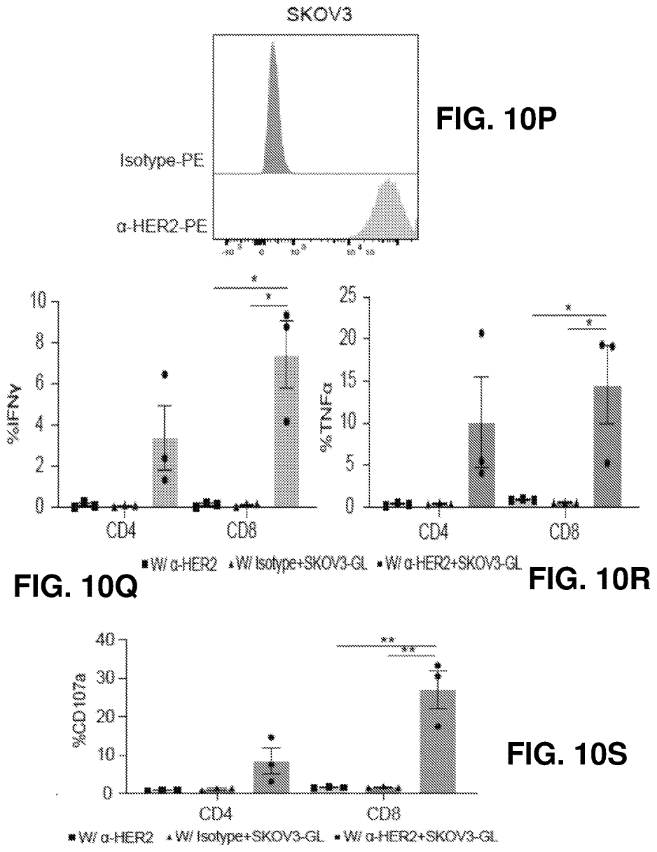

To show the versatility and modularity of GUAR-T cell therapy, multiple myeloma (MM), in which the expression of two major surface targets, B cell maturation antigen (BCMA) and G-protein coupled receptor family C group 5 member D (GPRC5D), have shown clinical signatures of patient survival (FIGS. 10A-10C), were targeted. An in-vitro co-culture system (FIGS. 3A and 10E) was established. GUAR-T cells were incubated with IgG1 anti-BCMA antibody or isotype control IgG1, and tumor clearance was titrated with different effector to target cell (E:T) ratios. The lysis results showed that GUAR-T-Clone2 cells showed potent and specific (antibody-dependent) lysis compared to control groups after 12 h of co-culture, which is consistent across all different E:T-ratios (FIG. 3B). To explore the correlation between effector function and target antibody concentration, different combinations of antibody concentration were titrated with indicated E:T ratios of GUAR-T-Clone2 and MM.1R-BCMA cells (FIG. 3C). Anti-BCMA antibody of as low as 0.01 μg/mL was still sufficient for GUAR-T-Clone2 cells' elimination of more than 80% MM.1R-BCMA after 12 h co-culture at E:T ratio of 1:1. and over 95% at E:T of 2:1 (FIG. 10F). These data indicated that GUAR-T cells can mediate sensitive antibody-dependent antigen-specific cancer killing.

The cytokine responses of GUAR-T-Clone2 was analyzed under exposure to MM.1R-BCMA with anti-BCMA antibody or isotype control, respectively. Upregulation of interferon-γ (IFNγ) and tumor necrosis factor α (TNFα) was observed after 6 h co-culture with MM.1R-BCMA under E:T ratio of 1:1 with anti-BCMA antibody compared to control groups, in both CD4 and CD8 GUAR-T-Clone2 cells generated from three different donors (FIGS. 3D-3E and 10G-10H). Moreover, expression of IFNγ and TNFα exhibited a dose-dependent manner with anti-BCMA antibody concentration from 0.01 μg/mL to 0.1 μg/mL (FIGS. 3D-3E). Degranulation was assessed through intracellular staining of CD107a. Both CD4 and CD8 GUAR-T-Clone2 cells showed enhanced degranulation upon stimulation by MM.1R-BCMA with anti-BCMA antibody relative to control groups, repeatable in T cells from three different donors (FIGS. 3F and 10I). Furthermore, increased degranulation of GUAR-T-Clone2 cells was observed with higher anti-BCMA antibody concentration when incubated with MM.1R-BCMA (FIG. 3F). Meanwhile, co-inhibitory receptor expression was analyzed in GUAR-T-Clone2 cells under exposure to MM.1R-BCMA with anti-BCMA antibody or isotype. Upregulated expression of LAG3, PD1, and TIM3 was observed after being stimulated by MM.1R-BCMA with anti-BCMA antibody in comparison to control groups in both CD4 and CD8 GUAR-T-Clone2 cells (FIGS. 10J-10O).

GPRC5D was next targeted because it has been reported as another promising target of MM therapeutics. A fully-human anti-GPRC5D antibody was developed, and this new binder was used to evaluate the surface expression of GPRC5D on MM cells (data not shown). GUAR-T-Clone2 and MM.1R-BCMA cells were set up in the same co-culture system under E:T ratio of 1:1 with human IgG1 antibody targeting GPRC5D or isotype control (FIG. 3G). The lysis results revealed that the anti-GPRC5D antibody can potently direct GUAR-T-Clone2 cells to lyse MM cells compared to control groups in an antibody-concentration dependent manner after 24 h of co-culture, which is further evident in the result of 72 h of co-culture (FIGS. 3H-3I). Together, these data indicated that stably knock-in GUAR-T cells have potent BCMA- or GPRC5D-targeting antibody-dependent anti-cancer functionality against MM cells.

### Example 4: GUAR-T Shows Potent In Vitro Function and In Vivo Efficacy Against Solid Tumor

### Results

GUAR-T cells' efficacy against solid tumors was investigated. SKOV3 is an ovarian cancer cell line with a high level of endogenous HER2 expression (FIG. 10P). To evaluate GUAR-T-Clone2's cancer-killing ability against SKOV3, GUAR-T-Clone2 and SKOV3 were set up in in vitro co-culture system with human IgG1 antibody targeting human HER2 or isotype control (FIG. 3J). Under E:T ratio of 1:1 and antibody concentration from 0.01 μg/ml to 1 μg/ml, GUAR-T-Clone2 mediated robust tumor cell killing in an antibody concentration-dependent manner (FIG. 3K). With antibody concentration at 0.01 μg/ml, GUAR-T-Clone2 mediated tumor cell killing efficacy increased from E:T ratio 1:2 to 2:1 after 24 h co-culture (FIG. 3L). Cytokine response was next analyzed through intracellular staining of interleukin-2 (IL-2), IFNγ, and TNFα. Compared to control groups, the levels of IL2, IFNγ, and TNFα were significantly upregulated in both CD4- and CD8-derived GUAR-T-Clone2 cells incubating with SKOV3 containing 0.1 μg/mL anti-HER2 antibody (FIGS. 3M-3O and 10Q-10R). Flow cytometry results showed significantly enhanced degranulation of both CD4 and CD8 GUAR-T-Clone2 cells after stimulation with SKOV3 and anti-HER2 antibody relative to control groups, repeatable in T cells from three different donors (FIGS. 3P and 10S).

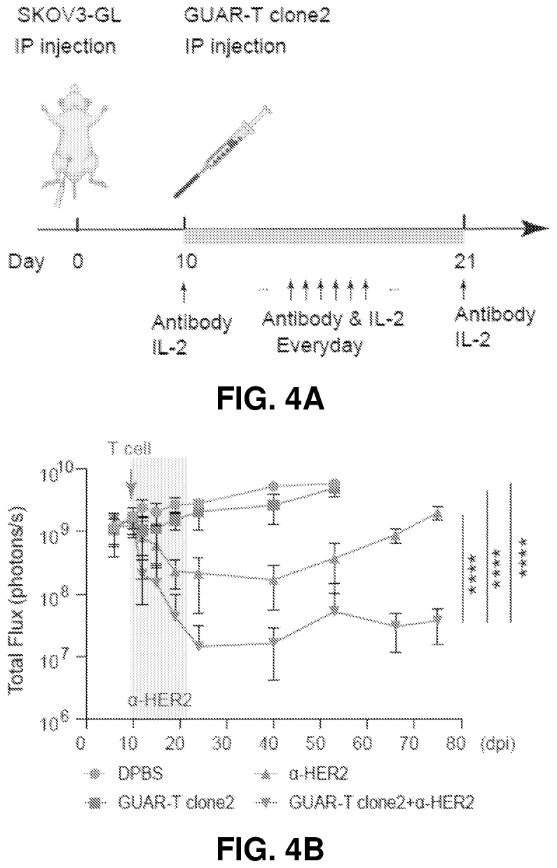

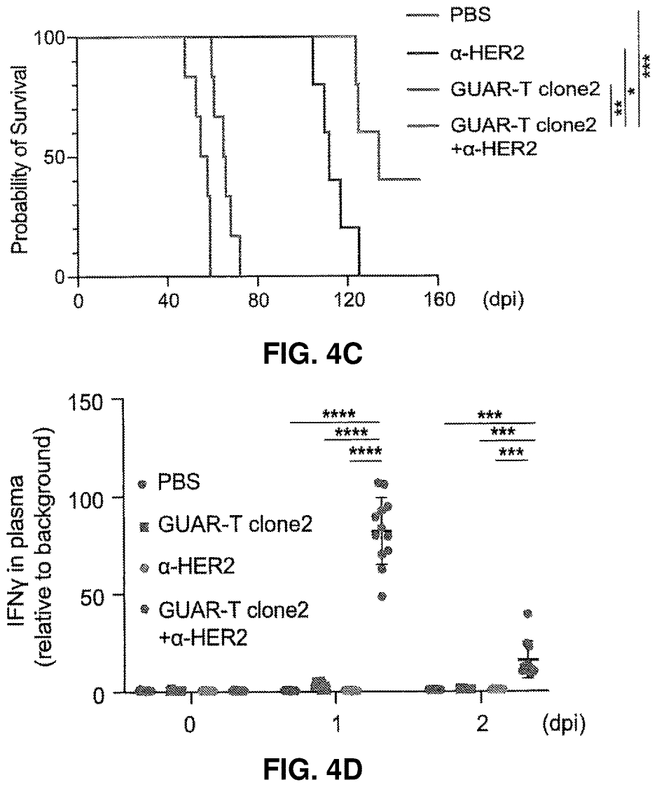

After validating the function of GUAR-T-Clone2 in vitro, its in vivo efficacy was investigated in a mouse cancer model. In this HER2-positive SKOV3 ovarian adenocarcinoma model, SKOV3 cells were intraperitoneally (i.p.) inoculated in NOD/SCID/IL-2Rγ-null (NSG) mice. Then GUAR-T-Clone2 cells were intraperitoneally infused on day 10 after tumor inoculation as a treatment. Anti-HER2 antibody was subsequently injected i.p. every day at 3 mg/kg for 12 days (FIG. 4A). Tumor growth was monitored by in vivo bioluminescence imaging (IVIS) of the luciferase signal from SKOV3 cancer cells in each mouse. GUAR-T-Clone2 with anti-HER2 antibody showed robust tumor burden clearance compared to control groups with GUAR-T-Clone2 cell alone or anti-HER2 antibody alone (FIG. 4B, mouse imaging data not shown). Prolonged survival was observed in mice treated with GUAR-T-Clone2 plus anti-HER2 antibody (FIG. 4C). Cytokine release in vivo was measured from plasma samples to verify that cytokine production is specific to the binding between anti-HER2 antibody and GUAR-T-Clone2. IFNγ was significantly increased in the plasma of mice treated with GUAR-T-Clone2 plus anti-HER2 antibody (FIG. 4D).

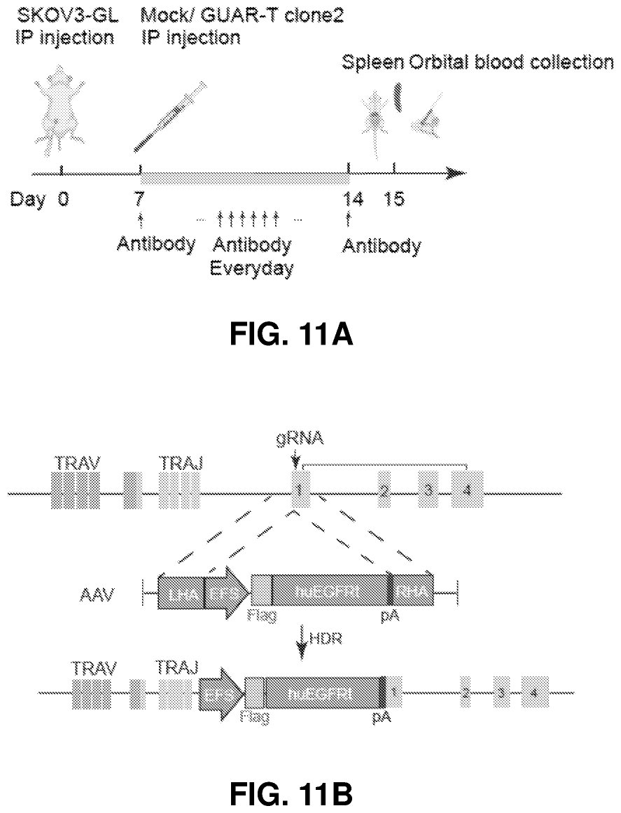

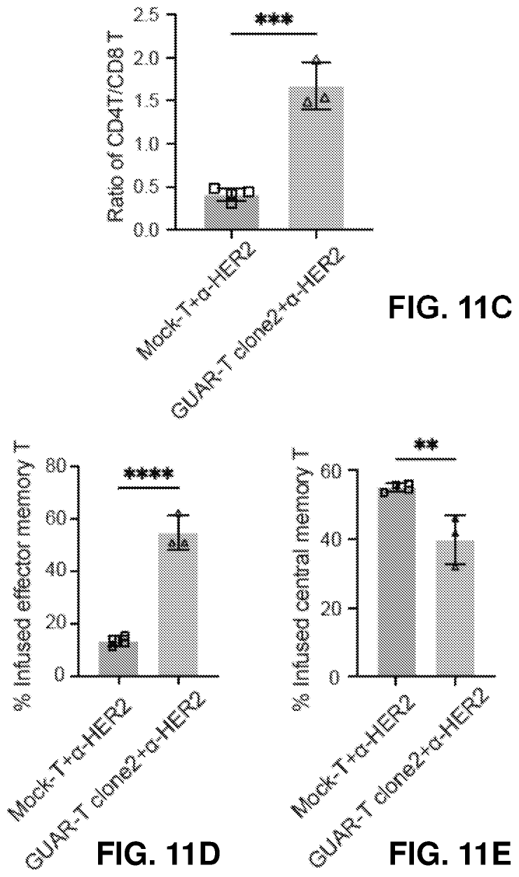

To further characterize infused GUAR-T-Clone2 cells, SKOV3 cells were inoculated through intraperitoneal injection, mock or GUAR-T-Clone2 cells were introduced, and anti-HER2 antibody were injected for 7 days before T cells collection from mouse spleen and orbital blood (FIG. 11A). Mock-T cells were generated by TRAC knock-in of truncated human EGFR polypeptide (huEGFRt) as previously reported (FIG. 11B). T cells were isolated from the mouse spleen for immunological analysis. Flow cytometry data showed that CD4 positive GUAR-T-Clone2 cell population was substantially enriched in the mouse spleen compared to the CD8 population (FIG. 11C). Moreover, significantly increased effector memory T cells were captured in the GUAR-T-Clone2 group relative to Mock-T cells, while the central memory T cell population was slightly decreased (FIGS. 11D-11E). Overall, with target-specific therapeutic antibodies, GUAR-T-Clone2 elicits efficient cancer clearance in a solid tumor model both in vitro and in vivo.

### Example 5: Evaluation of Different Signaling Domains and Promoters in GUAR-T

### Results

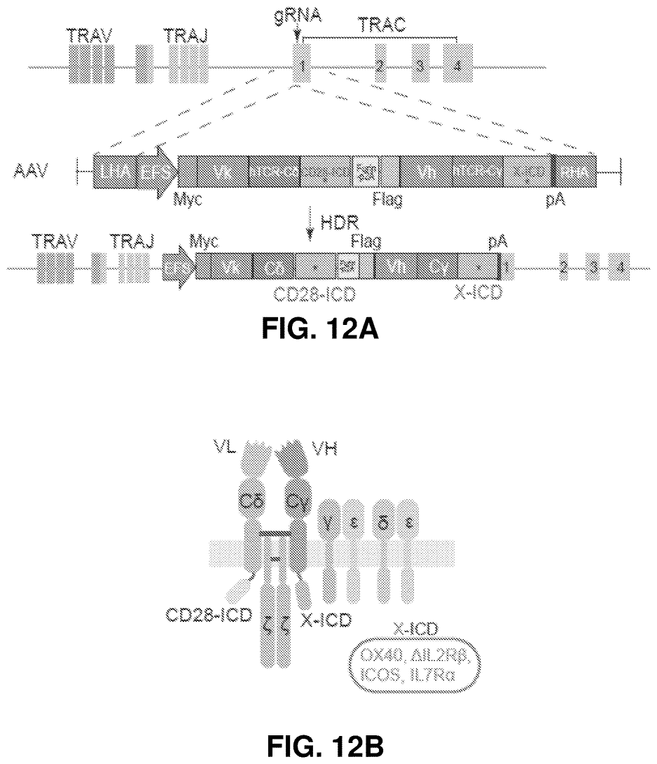

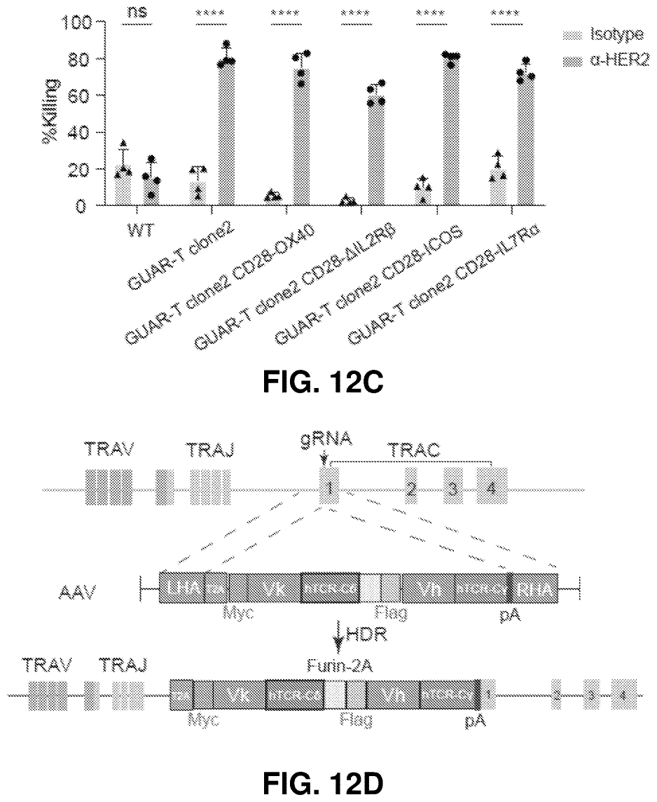

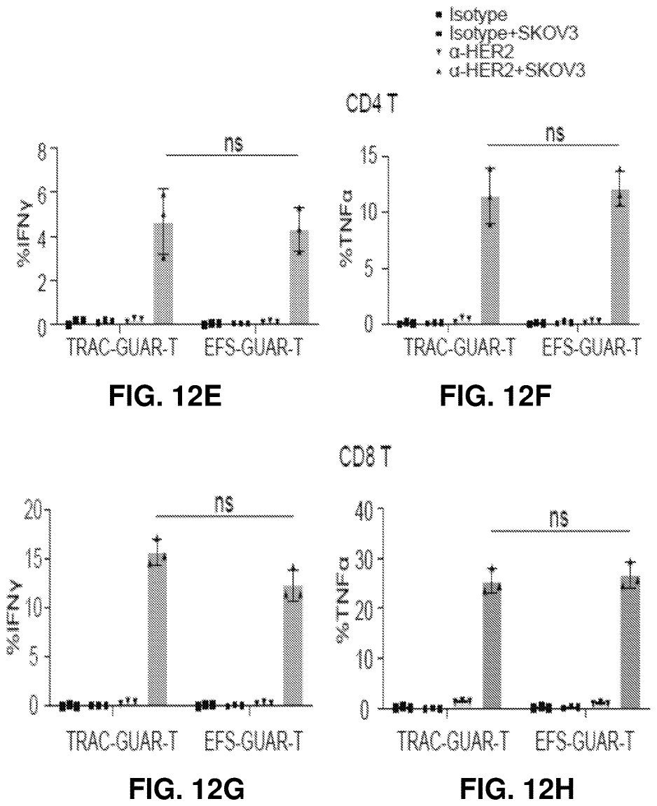

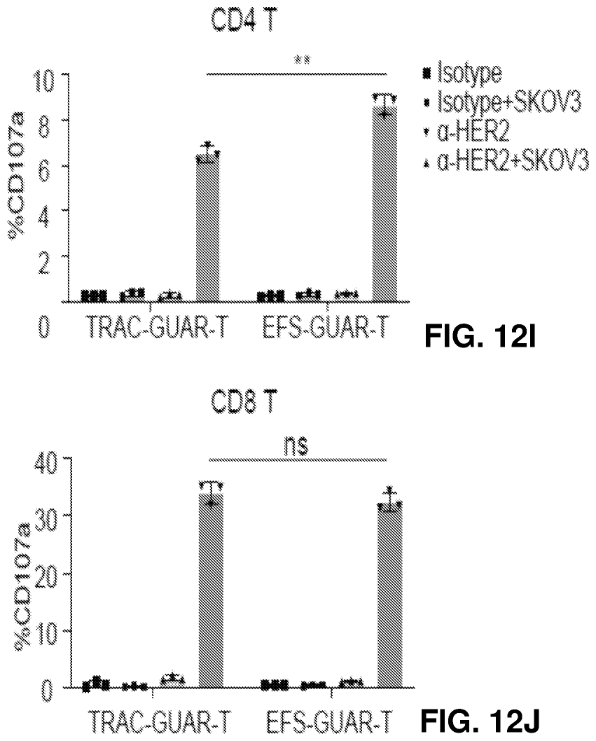

To test how different signaling domains compare in modulating GUAR-T-Clone2 function, the costimulatory domain or intracellular domain from the cytokine receptor was reconstituted to the C terminal of GUAR-Clone2 construct. Since there are TCR γ chain and δ chain, CD28 intracellular domain was fixed to the C-terminal of the TCR δ chain, and generated a series of constructs where the intracellular domain of OX40, ICOS, IL7Rα, or ΔIL2Rβ was fused to C-terminal of TCR γ chain, respectively (FIGS. 12A-12B). Surface staining illustrated that all the remodulated GUAR-Clone2 receptors efficiently assemble with the CD3 complex and express on the T cell surface (data now shown). To assess the effector function of remodulated GUAR-T-Clone2 cells, a transduced T cell and SKOV3 cancer cell co-culture assay was established with anti-HER2 antibody as an adaptor. Compared to isotype control, all the remodulated T cells showed efficient tumor cell killing efficacy with 0.01 μg/ml anti-HER2 antibody at an E:T ratio of 1:2 (FIG. 12C). These data showed that all five versions of signaling domain constructs can mediate effective and functional GUAR-T activity.

Targeting CAR to the TRAC locus can utilize its transcriptional control. To evaluate the GUAR-Clone2 function driven by the promoter of TRAC as compared to EFS, a TRAC-GUAR-Clone2 encoding a self-cleaving T2A peptide followed by the GUAR-Clone2 cDNA was constructed (FIG. 12D). TRAC-GUAR-Clone2 was integrated into the TRAC locus in primary human CD3 T cells using CRISPR-Cas9 mediated targeted knock-in. Surface staining of FLAG tag and CD3 indicated that TRAC-GUAR-Clone2 efficiently co-express with CD3 complex in human T cells. According to the MFI, the surface expression of TRAC-GUAR-Clone2 was relatively lower than the EFS-driven GUAR-Clone2 (EFS-GUAR-Clone2) (data not shown). Furthermore, the cytokine release of TRAC promoter or EFS promoter driven GUAR-T-Clone2 cells was analyzed upon stimulation with HER2 expressing SKOV3 cancer cells. Compared with antibody-only or co-culture with the isotype control group, both EFS- and TRAC-driven GUAR-T-Clone2 cells showed substantially increased IFNγ and TNFα secretion upon co-culture with anti-HER2. However, there is no significant difference between EFS and TRAC-driven GUAR-T-Clone2 cells (FIGS. 12E-12H). For degranulation, enhanced CD107a expression was observed in EFS- and TRAC-GUAR-T-Clone2 cells stimulated by SKOV3 with anti-HER2 antibody relative to control. Meanwhile, TRAC-GUAR-Clone2 expressing CD4 T cells exhibited slightly lower CD107a expression in comparison to EFS-GUAR-Clone2 carrying CD4 T cells upon in vitro cancer stimulation (FIGS. 12I-12J). While in the CD8 T cell population, a similar degranulation level was observed in TRAC- and EFS-GUAR-Clone2 T cells. These data showed that both versions of promoters (EFS and TRAC) can drive GUAR expression and effector function in human T cells.

### Example 6: AlphaFold2-Multimer Structure Prediction of GUAR-Clone2-IgG1-Fc Complexes and Structure-Guided Functional Optimization

### Results

To characterize the molecular basis of the epitope and paratope of the GUAR-Clone2 in complex with a hIgG1-Fc protein, AlphaFold2-Multimer (AF2-multimer, version 2.2.4) was used to model the structural conformations of the complexes. The hIgG1-Fc fragment exhibited a 1:1 complex in both selected conformations with the GUAR-antibody receptor domains but displayed different conformational states. In conformation 1, the major interface area of the complex was identified that between one side chain of hIgG1-Fc fragment and GUAR-antibody receptor domain, and seven potential hydrogen bonds were formed by IgG1-Fc-L36 with GUAR VH-G101 (with 2.3 Å), IgG1-Fc-Q127 with GUAR VH-S30 (with 1.8 Å and 1.9 Å), IgG1-Fc-E130 with GUAR VH-S54 (with 2.5 Å) and VH-T56 (with 1.6 Å), IgG1-Fc-N219 with GUAR VK-S91 (with 1.8 Å) and VH-G100 (with 1.9 Å). The major interface area of the complex in conformation 2 was identified that between end domains of hIgG1-Fc fragment and GUAR-antibody receptor domain, where nine potential hydrogen bonds were formed by IgG1-Fc-chain1-G231 with GUAR VK-Q27 (with 1.9 Å), IgGt-Fc-chain1-K232 with GUAR VK-A25 (with 1.8 Å), VK-G68 (with 2.7 Å), and VK-S93 (with 1.6 Å), IgG1-Fc-chain2-R140 with GUAR VK-Y32 (with 2.1 Å), IgG1-Fc-chain2-Q203 with GUAR VH-Y52 (with 2.8 Å), IgG1-Fc-chain2-G231 with GUAR VH-N58 (with 1.9 Å), IgG1-Fc-chain2-K232 with GUAR VK-T94 (with 2.1 Å) and VH-N58 (with 2.2 Å).

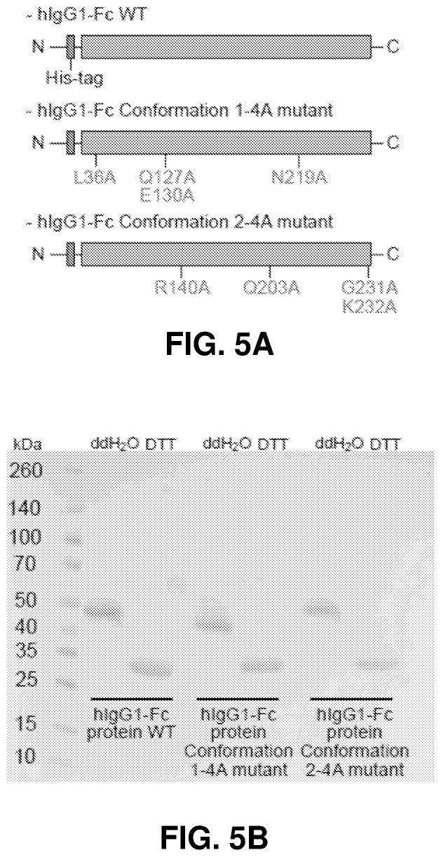

To further validate the binding sites of the hIgG1-Fc fragment, an alanine scanning mutagenesis approach was performed on the hIgG1-Fc fragment according to estimated interface residues in the above conformations, and termed hIgG1-Fc-conformation 1-4A mutant (L36A; Q127A; E130A; N219A) and hIgG1-Fc-conformation 2-4A mutant (R140A; Q203A; G231A; K232A) (FIG. 5A). Mutants were expressed and purified together with WT Fc proteins. The results of Coomassie-stained SDS-PAGE revealed that alanine mutations did not affect mutated hIgG1-Fc protein assembly and purification (FIG. 5B). Flow cytometry results showed that conformation 2-based hIgG1-Fc-4A mutant abolished the binding to GUAR-Clone2-J cells, whereas conformation 1-based hIgG1-Fc-4A mutant maintained strong binding at the same level as WT IgG1-Fc protein, indicating that the AFM conformation-2 of GUAR-Clone2-IgG1-Fc complex identified residues important for GUAR-Clone2:IgG1-Fc binding (data not shown). To obtain an improved model on current conformations architecture, the latest AF2-multimer (version 2.3.0) was applied, which showed higher accuracies than version 2.2.4, to establish complex structures. In the conformation-2 established by v2.3.0 AF2-multimer, the 4 key interface residues (R140; Q203; G231; K232) were confirmed, along with other potential interface residues. In addition, to evaluate whether swapping constant domains affected GUAR cross-reactivity, the interaction of GUAR-Clone2-J cells with all four human IgG-Fc proteins were tested. The results showed that GUAR-Clone2-J cells retain the potency to recognize most human IgG-Fc proteins including IgG1, IgG2, and IgG3, but not IgG4 (data not shown).

### Example 7: Design and Characterization of GUAR Masks

### Results

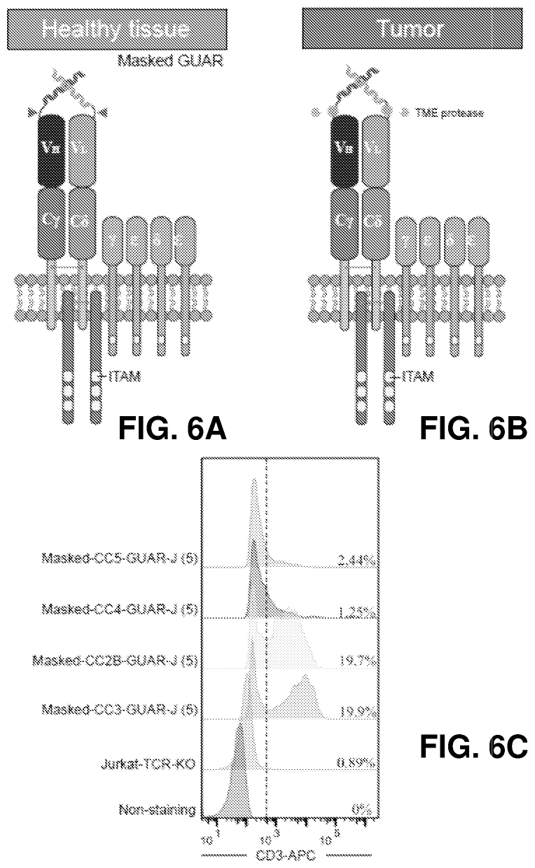

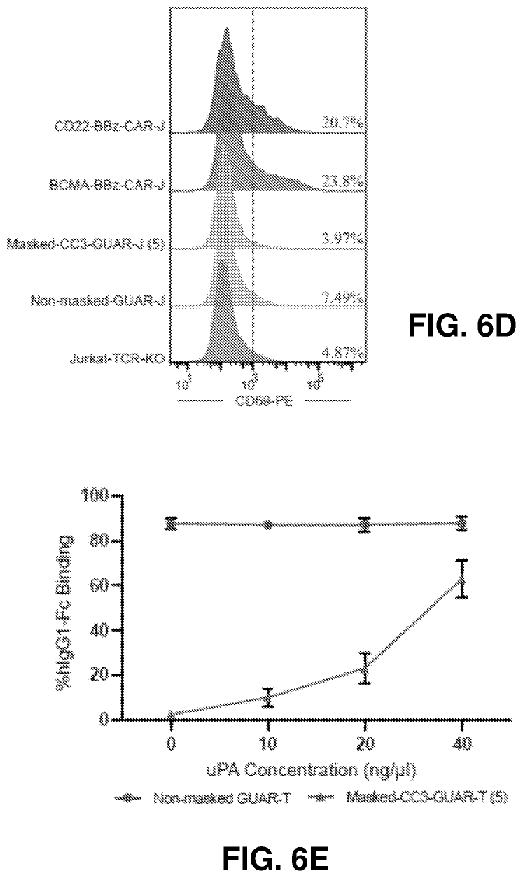

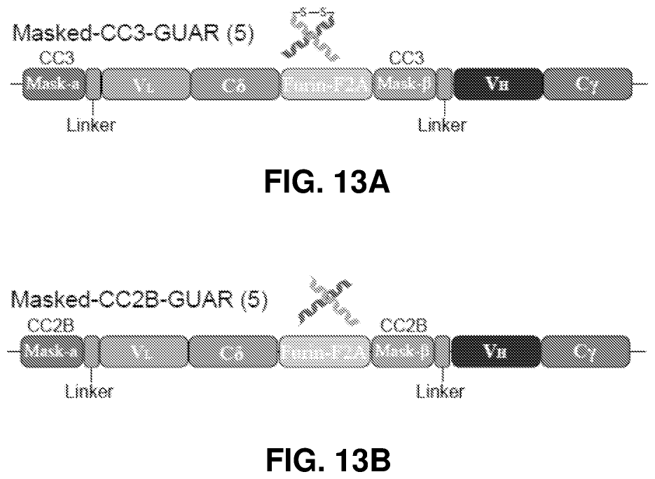

To reduce cytotoxic killing due to potential on-target, off-tumor activities masking was explored. mAb-mediated immunotherapy can exert cytotoxicity by activation of Fc receptors expressed in immune cells, such as NK cells, neutrophils, and macrophages. However, various levels of side effects have been observed in clinical trials after mAbs administration. Thus, it is important to develop a tumor-selective GUAR-Clone2 approach that can trigger therapeutic effects on the tumor site while sparing healthy tissues and circulation. One potential approach is fusing a masking group to GUAR's antibody receptor domain using a protease-cleavable mask, which can be released upon encountering tumor-associated proteases in the tumor microenvironment (TME), similar to validated masked antibody therapies. Experiments were designed to test if the fusion of leucine zipper coiled-coils upstream of GUAR-Clone2 (termed masked GUAR), would render GUAR-engineered T cells inactive, and selectively re-activeable upon proteolytic cleavage with TME-specific proteases (FIGS. 6A-6B). As a proof of concept, GUAR constructs were designed with several types of masking coiled-coil peptides (CC2B, CC3, CC4, and CC5) based on the mask design in a previous report, and fused them to the N-terminal of GUAR-Clone2 construct with protease-cleavable linkers that contained a urokinase plasminogen activator (uPA, a serine protease (SP))-sensitive sequence (LSGRSDNH) with a GSSGT spacer, namely masked GUAR (5) (5 refers to the numbers of amino acid in the GS spacer) (FIGS. 13A-13B).

These several masked GUAR candidates (5) were evaluated, by introducing them into TCR-αβ-KO Jurkat cells via lentiviral vectors, and examining the TCR-CD3 complex formation by FACS after 72 hours of transduction. The results showed that only masked-CC3 and masked-CC2B GUAR-J (5) cells had substantial level of surface TCR-CD3 complex formation (~20% of TCR-CD3 positive cells), but not on masked-CC4 and masked-CC5 transduced cells (FIG. 6C). To assess the binding capacity of masked GUAR-J (5) cells in the presence of target antigen, these CD3 positive masked cells were further enriched, respectively, and co-incubated with biotinylated hIgG-Fc proteins followed by staining with streptavidin PE-labeled antibody. Nearly 100% of enriched non-masked GUAR-Clone2-J cells showed binding to hIgG1-Fc protein (data not shown). Both enriched masked-CC3 and masked-CC2B GUAR-J (5) cells exhibited robust antigen-blocking ability, with only 1.72% (masked-CC3) and 3.51% (masked-CC2B) baseline binding detected, respectively. Proteolytic cleavage of the coiled-coil masks was examined by treating them with recombinant uPA, and testing whether cleavage restores binding by FACS analysis after incubation with the target protein. The results showed that the binding ability of both de-masked GUAR-J (5) cells was gradually restored in a dose-dependent manner upon uPA treatment, and a high concentration of uPA treatment also did not affect the binding ability of non-masked GUAR-Clone2-J cells (data not shown).

To enhance proteolytic cleavage of masked molecules, different lengths of cleavable linker were tested. Two different lengths of cleavable linkers were designed, namely masked GUAR (0) and masked GUAR (8) (GS spacer was replaced by SP-sensitive sequence), and examined by blocking activity and cleavage-dependent restoration of binding via FACS analysis. The results indicated that if the GS spacer had more than 8aa, then the blocking activity of both masked-CC3 and masked-CC2B GUAR-J (8) cells was substantially reduced (data not shown). On the contrary, if there was no GS spacer between the mask and GUAR-binding domains, then there is no binding of de-masked GUAR-J (0) cells upon any concentration of uPA treatment (data not shown). Therefore, the masked-CC3 GUAR (5) construct was selected for further characterization.

### Example 8: GUAR Masking Reduces Tonic Signaling and Inhibits Antigen Stimulated-Activation Absent of Proteases

### Results

Antigen-independent tonic signaling has been widely reported to result in CAR-T cell exhaustion and dysfunction. However, TCR-like T-cell therapy has taken its natural advantage in hierarchical signaling delivery, which has been demonstrated to not induce tonic signaling under antigen-free conditions. Autoactivation levels of GUAR-J cells was examined and compared with BBzCAR-J cells by detecting CD69 expression under antigen-free conditions. The results revealed that both non-masked GUAR-J cells and masked-CC3 GUAR-J (5) cells expressed a similar level of CD69 to its parental Jurkat cells, whereas BBzCAR-J cells showed 5-6-fold higher CD69 expression compared with its parental Jurkat cells. Furthermore, to better understand the conformation of the masked-CC3 GUAR (5) construct, AlphaFold was again used to model the structural conformation with high-confidence (FIG. 6D).

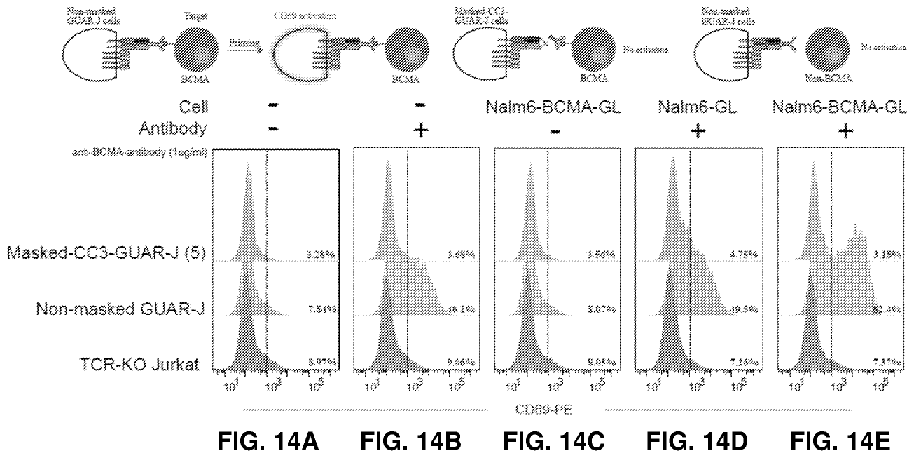

Given the validated blocking properties of masked-CC3 GUAR, experiments were designed to test if the masked-CC3 peptides could tightly inhibit the antigen-stimulated activation of masked GUAR-J (5) cells in the absence of proteases. To investigated this, masked-CC3 GUAR-J cells were incubated with various stimulation conditions, together with non-masked GUAR-J cells and parental Jurkat cells, and monitored the activation status by detecting CD69 up-regulation (FIGS. 14A-14E). The results demonstrated that masked-CC3 GUAR-J cells have exhibited tight inhibitory self-control upon antigen stimulation with the absence of proteases. In contrast, non-masked GUAR-J cells triggered different levels of CD69 up-regulation after stimulation with either plate-attached immobilized antibody or antigen-expressing cell-attached immobilized antibody (FIGS. 14A-14E). The masked-CC3 GUAR (5) construct was introduced into three different human donors' primary T cells, and measured the blocking activity and cleavage restored binding via FACS analysis, respectively. The results showed that masked-CC3 GUAR-T (5) cells showed strong antigen-blocking ability in the absence of proteases and restored binding in a dose-dependent manner in the presence of proteases (FIG. 6E). These data showed that masking is compatible with GUAR in T cells and provide more refined control of activity.

### Example 9: Genome Editing of the Allogeneity-Relevant Loci of GUAR-T in Both αβ T and γδ T Cells

### Results

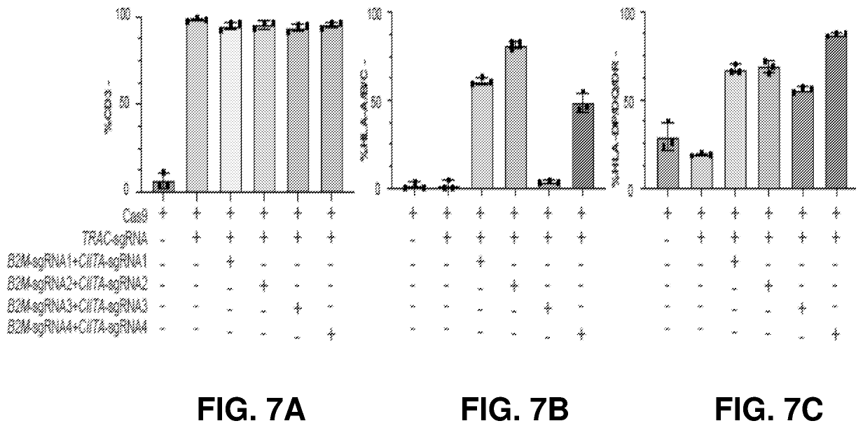

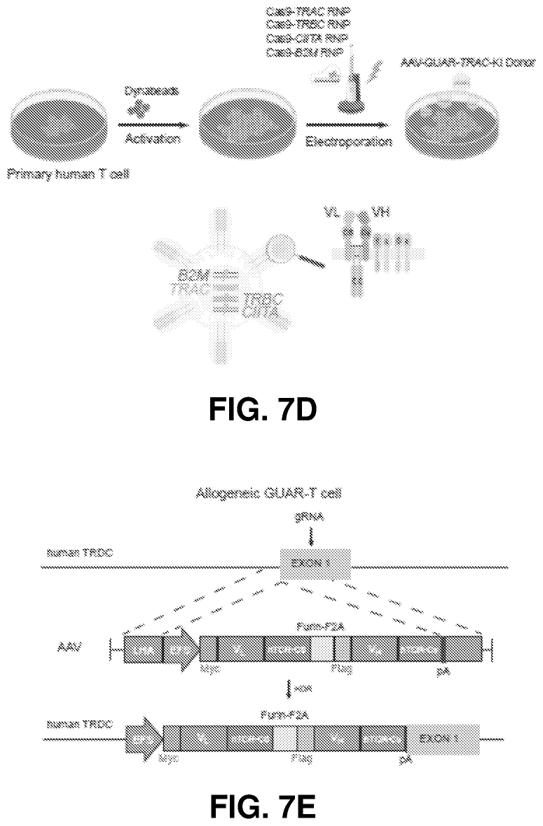

Towards generation of allogeneic GUAR-T cells, TRAC and TRBC were targeted to eliminate TCRαβ surface expression, while B2M and CIITA were ablated to abrogate HLA class I and class II molecules. Several sgRNAs targeting B2M and CIITA (FIGS. 7A-7C) were tested. From flow cytometry data, editing of TRAC in all groups resulted in over 90% efficiency in TCR complex ablation in ab T cells (FIGS. 7A-7C). Quantification of surface expression of HLA-A/B/C and HLA-DP/DQ/DR showed that B2M targeting sgRNA-2 and CIITA targeting sgRNA-4 had the highest efficiency (FIGS. 7A-7C). Next, electroporation of Cas9-RNPs targeting TRAC, TRBC, B2M, and CIITA was performed and AAV was used as a template to integrate GUAR-T-Clone2 construct into TRAC locus (FIG. 7D). Five days after electroporation, the surface expression of HLA-A/B/C, HLA-DP/DQ/DR, and TCRαβ on GUAR-T Clone2 cells was analyzed by flow cytometry. Notably, 96.6% of GUAR-T Clone2 cells were HLA-A/B/C and HLA-DP/DQ/DR double negative and nearly all GUAR-T Clone2 cells were TCRαβ negative (data now shown). These data showed that GUAR-T cells with editing of multiple allogeneity-relevant genes can be efficiently generated in primary human αβ T cells.

γδ T cell is another source for off-the-shelf adoptive T cell therapy given that most γδ T cells recognize target cells independently of HLA antigen presentation thus low risk of GvHD. To characterize GUAR-T Clone2 in γδ T cells, first γδ T cells were expanded from human peripheral blood mononuclear cells (PBMCs) with zoledronic acid (ZA). Seven days after ZA treatment, the γδ T cell population was analyzed by surface staining of TCR γδ expression. Compared to day 0, γδ T cells were enriched from 3.1% to 37.4%, indicating the successful expansion upon ZA treatment (data now shown). Moreover, CD3 expression was highly correlated with TCR γδ expression on the expanded γδ T cells. Then, γδ T cells were purified by flow cytometry and further used for GUAR-γδT generation. Corresponding to TRAC locus knock-in on αβ T cells, GUAR-T-Clone2 construct was integrated into TRDC in γδ T cells by electroporation Cas9-RNP targeting the exon1 of TRDC and AAV was used as a template to mediate knock-in. Five days after electroporation, 41.9% of GUAR-Clone2 expressing γδ T cells were detected by surface expression of FLAG. Moreover, CD3 expression was correlated with GUAR-Clone2, indicating efficient assembly of GUAR-Clone2 and CD3 complexes in γδ T cells. These data demonstrated that GUAR-Clone2 can be stably integrated into γδT cells.

Harnessing a combination of chimeric γδTCR and antibody recognition mechanisms, a universal, switchable cell therapy platform was developed. This platform, term Gamma delta TCR signaling directed Universal Antibody Receptor (GUAR) T cell therapy, engineers TCR γ/δ chain constant regions with variable regions of a custom anti-hIgG1-Fc antibody heavy and light chains, respectively. GUAR-expressing T cells (GUAR-Ts) recognize the Fc-portion of therapeutic antibodies, which enable the selective targeting of different antigens on demand with different therapeutic monoclonal antibodies, and maintain potent and sensitive signaling capacity via γδTCR signaling. Stable knock-in GUAR-T cells showed potent antibody-dependent activity and in vivo anti-tumor efficacy. Given the repertoire of rapidly increasing therapeutic antibodies, GUAR-T opens immense potential as a highly adaptive and universal cell therapy.

To date, CAR-T cell therapy has been demonstrated to be a potent therapeutic approach in the treatment of certain hematological malignancies, with six CAR-T cell products currently approved for clinical use by the FDA. Nevertheless, high rates of therapeutic resistance and post-treatment tumor recurrence remain as primary limitations. It has been reported that almost half of the pre-B cell ALL patients observed tumor relapse within 12 months after receiving anti-CD19 or anti-CD22 CAR-T cells. Surprisingly, tumor recurrence is also detected even in dual-targeted CAR-T treatments, indicating that tumor antigen loss is a major obstacle hindering the application of current CAR-T therapy.

To overcome the limitation of tumor antigen loss, certain modular CAR-T platforms are being tested. Universal modular CAR-T cells can logically target multiple antigens by switching different CAR-adaptor molecules, and thereby control the overactivation of CAR-T to reduce undesired side effects by withdrawal of the CAR-adaptor molecules. Most importantly, universal CAR-T cells allow expansion without additional engineering needed, which can dramatically reduce cost towards “off-the-shelf” T cell products for patients with tumor relapse. However, the same as most of clinically used single antigen-specific CAR-T cells, existing modular CAR-T cells commonly use regular CAR signaling and are generated by lentiviral/retroviral-associated methods, resulting in high levels of CAR expression on the T cell surface that can induce spontaneous CAR-clustering in an antigen-independent manner, promoting tonic signaling and leading CAR-T cells to early exhaustion. In contrast to CARs, TCRs are naturally evolved machinery with sophisticated regulatory mechanism that allow TCR-T cells to be less prone to tonic signaling, have less exhausted phenotype and better hierarchical T cell responses. Thus, TCR-T cell therapy have shown promising effectiveness and persistence in the treatment of solid tumors, but its applications are still restricted to MHC proteins of certain HLA alleles, or the availability of antigen-specific TCRs.

Several adaptable chimeric receptors have been developed, which allow recognition of multiple tumor antigens by immune cells expressing a single receptor. The design of universal CARs hinges on the separation of targeting and signaling modules. An adaptor serves as the targeting element that binds to tumor associated antigen, and is required to bridge CAR-T stimulation. Different format of the adaptor has led to the development of distinct CAR design. Tag- and anti-tag-specific universal CARs were reported to mediate specific cancer cell killing by using biotinylated or FITC-tagged molecules, leucine zipper et al as adaptors. Other designs include bispecific protein-engaging CARs, which engage T cells and tumor simultaneously to stimulate T cell effector function. Engineered binders such as scFvs are used in cell therapy to mediate specific cancer targeting. However, extensive antibody engineering is time and labor-consuming, especially for the wide repertoire of antibody-dependent cellular cytotoxicity (ADCC)-mediating monoclonal antibodies already in clinical grade quality. In comparison, Fc-binding adaptor CARs are generated to take advantage of the well-investigated interaction between CD16 and the Fc part of IgG molecules, which, in combination with tumor-specific monoclonal antibodies, can trigger tumor lysis.

Recently, two TCR-based chimeric antigen receptors were developed, using either mouse αβTCR or human αβTCR constant region fused with the variable region from Fab (STAR-T and HIT-T). Compared to CAR, these TCR-based chimeric antigen receptor mediates strong and sensitive TCR-like signaling, which shows higher antigen sensitivity. However, one of the challenges in TCR-based cell therapy is TCR mispairing—i.e. the incorrect pairing between an introduced TCR α or β chain and an endogenous TCR β or α chain, which results in diluted surface expression of the engineered TCRαβ as well as potential self-reactivity. The STAR-T replaced human TCR constant regions with mutant murine counterparts, while HIT-T targeted the TRAC locus in T cells to reconstruct the antigen binding domain of the heterodimeric TCR. However, murine TCR may lead to potential immunogenicity, while TRAC locus knockin of engineered TCR may still compete with the β chain of the endogenous TCR for expression. Of note, neither STAR-T nor HIT-T are universal adaptor based cell therapy.

Herein provide is a platform, as referred to as GUAR-T, which is as a universal and potent form of cell therapy. GUAR is designed to specifically recognize Fc domains of human IgG antibodies that allow flexible adaptation of diverse IgG-based therapeutic antibodies, and engage endogenous CD3 signaling machinery from γδ TCR subunits. In addition, the conformation of GUAR closely mimics a native TCR both in size and structure, which could potentially decrease unwanted tonic signaling, and integrated GUAR into the TRAC locus that has been demonstrated to improve efficacy and reduce toxicity (86). Results presented herein revealed that GUAR-T cells exhibited rapid activation and effectively lysed target cells through the lower density of IgG antibodies, taking advantage of natural signaling of γδ TCR-CD3 complexes that naturally requires fewer numbers of TCR ligands to be engaged for cytolysis. The experiments exemplify GUAR into two different T cell subtypes (CD4⁺ and CD8⁺) and demonstrate that GUAR receptors can independently regulate two cell types and release different levels of cytokines and degranulation. GUAR-T cells have potent efficacy in a solid tumor mouse model in an antibody-specific manner. Given the available and evolving FDA-approved clinical antibodies and emerging clinical candidates in development, GUAR-T system offers a universal and versatile form of plug-and-play antibody-cell therapy.

### REFERENCES

1. C. H. June, R. S. O'Connor, O. U. Kawalekar, S. Ghassemi, M. C. Milone, CAR T cell immunotherapy for human cancer. Science 359, 1361-1365 (2018).
2. S. S. Neelapu et al., Axicabtagene Ciloleucel CAR T-Cell Therapy in Refractory Large B-Cell Lymphoma. N Engl J Med 377, 2531-2544 (2017).
3. S. L. Maude et al., Tisagenlecleucel in Children and Young Adults with B-Cell Lymphoblastic Leukemia. N Engl J Med 378, 439-448 (2018).
4. J. S. Qin et al., Antitumor Potency of an Anti-CD19 Chimeric Antigen Receptor T-Cell Therapy, Lisocabtagene Maraleucel in Combination With Ibrutinib or Acalabrutinib. J Immunother 43, 107-120 (2020).
5. J. G. Berdeja et al., Ciltacabtagene autoleucel, a B-cell maturation antigen-directed chimeric antigen receptor T-cell therapy in patients with relapsed or refractory multiple myeloma (CARTITUDE-1): a phase 1b/2 open-label study. Lancet 398, 314-324 (2021).
6. N. C. Munshi et al., Idecabtagene Vicleucel in Relapsed and Refractory Multiple Myeloma. N Engl J Med 384, 705-716 (2021).
7. M. K. Anderson, A. Torosyan, Z. Halford, Brexucabtagene Autoleucel: A Novel Chimeric Antigen Receptor T-cell Therapy for the Treatment of Mantle Cell Lymphoma. Ann Pharmacother 56, 609-619 (2022).
8. P. Ren et al., CAR-T Therapy in Clinical Practice: Technical Advances and Current Challenges. Adv Biol (Weinh) 6, e2101262 (2022).
9. U. Patel et al., CAR T cell therapy in solid tumors: A review of current clinical trials. E J Haem 3, 24-31 (2022).
10. S. Chacim et al., Costs, effectiveness, and safety associated with Chimeric Antigen Receptor (CAR) T-cell therapy: Results from a comprehensive cancer center. PLoS One 17, e0278950 (2022).
11. X. Xu et al., Mechanisms of Relapse After CD19 CAR T-Cell Therapy for Acute Lymphoblastic Leukemia and Its Prevention and Treatment Strategies. Front Immunol 10, 2664 (2019).
12. L. Vercellino et al., Predictive factors of early progression after CAR T-cell therapy in relapsed/refractory diffuse large B-cell lymphoma. Blood Adv 4, 5607-5615 (2020).
13. S. Guedan, H. Calderon, A. D. Posey, Jr., M. V. Maus, Engineering and Design of Chimeric Antigen Receptors. Mol Ther Methods Clin Dev 12, 145-156 (2019).
14. J. Mikhael, J. Fowler, N. Shah, Chimeric Antigen Receptor T-Cell Therapies: Barriers and Solutions to Access. JCO Oncol Pract 18, 800-807 (2022).
15. E. Zah, M. Y. Lin, A. Silva-Benedict, M. C. Jensen, Y. Y. Chen, T Cells Expressing CD19/CD20 Bispecific Chimeric Antigen Receptors Prevent Antigen Escape by Malignant B Cells. Cancer Immunol Res 4, 498-508 (2016).
16. D. Schneider et al., A tandem CD19/CD20 CAR lentiviral vector drives on-target and off-target antigen modulation in leukemia cell lines. J Immunother Cancer 5, 42 (2017).
17. D. Schneider et al., Trispecific CD19-CD20-CD22-targeting duoCAR-T cells eliminate antigen-heterogeneous B cell tumors in preclinical models. Sci Transl Med 13, (2021).
18. A. R. Sutherland, M. N. Owens, C. R. Geyer, Modular Chimeric Antigen Receptor Systems for Universal CAR T Cell Retargeting. Int J Mol Sci 21, (2020).
19. N. Chen, X. Li, N. K. Chintala, Z. E. Tano, P. S. Adusumilli, Driving CARs on the uneven road of antigen heterogeneity in solid tumors. Curr Opin Immunol 51, 103-110 (2018).
20. J. H. Cho et al., Engineering advanced logic and distributed computing in human CAR immune cells. Nat Commun 12, 792 (2021).
21. J. H. Cho, J. J. Collins, W. W. Wong, Universal Chimeric Antigen Receptors for Multiplexed and Logical Control of T Cell Responses. Cell 173, 1426-1438 e1411 (2018).
22. S. Caratelli et al., FCgamma Chimeric Receptor-Engineered T Cells: Methodology, Advantages, Limitations, and Clinical Relevance. Front Immunol 8, 457 (2017).
23. M. Poorebrahim et al., Counteracting CAR T cell dysfunction. Oncogene 40, 421-435 (2021).
24. T. Kouro, H. Himuro, T. Sasada, Exhaustion of CAR T cells: potential causes and solutions. J Transl Med 20, 239 (2022).
25. D. Gumber, L. D. Wang, Improving CAR-T immunotherapy: Overcoming the challenges of T cell exhaustion. EBio Medicine 77, 103941 (2022).
26. A. H. Long et al., 4-1BB costimulation ameliorates T cell exhaustion induced by tonic signaling of chimeric antigen receptors. Nat Med 21, 581-590 (2015).
27. J. Fisher et al., Engineering gammadeltaT cells limits tonic signaling associated with chimeric antigen receptors. Sci Signal 12, (2019).
28. A. Ajina, J. Maher, Strategies to Address Chimeric Antigen Receptor Tonic Signaling. Mol Cancer Ther 17, 1795-1815 (2018).
29. P. Rodriguez-Marquez et al., CAR density influences antitumoral efficacy of BCMA CAR T cells and correlates with clinical outcome. Sci Adv 8, eabo0514 (2022).
30. C. S. Guy et al., Distinct TCR signaling pathways drive proliferation and cytokine production in T cells. Nat Immunol 14, 262-270 (2013).
31. Y. Liu et al., Chimeric STAR receptors using TCR machinery mediate robust responses against solid tumors. Sci Transl Med 13, (2021).
32. N. Singh et al., T cells targeting NY-ESO-1 demonstrate efficacy against disseminated neuroblastoma. Oncoimmunology 5, e1040216 (2016).
33. A. P. Rapoport et al., NY-ESO-1-specific TCR-engineered T cells mediate sustained antigen-specific antitumor effects in myeloma. Nat Med 21, 914-921 (2015).
34. H. Torikai et al., A foundation for universal T-cell based immunotherapy: T cells engineered to express a CD19-specific chimeric-antigen-receptor and eliminate expression of endogenous TCR. Blood 119, 5697-5705 (2012).
35. D. Stenger et al., Endogenous TCR promotes in vivo persistence of CD19-CAR-T cells compared to a CRISPR/Cas9-mediated TCR knockout CAR. Blood 136, 1407-1418 (2020).
36. R. Benjamin et al., UCART19, a first-in-class allogeneic anti-CD19 chimeric antigen receptor T-cell therapy for adults with relapsed or refractory B-cell acute lymphoblastic leukaemia (CALM): a phase 1, dose-escalation trial. Lancet Haematol 9, e833-e843 (2022).
37. J. Eyquem et al., Targeting a CAR to the TRAC locus with CRISPR/Cas9 enhances tumour rejection. Nature 543, 113-117 (2017).
38. The Quest for Off-the-Shelf CAR T Cells. Cancer Discov 8, 787-788 (2018).
39. X. Liu et al., CRISPR-Cas9-mediated multiplex gene editing in CAR-T cells. Cell Res 27, 154-157 (2017).
40. J. Tipanee, E. Samara-Kuko, T. Gevaert, M. K. Chuah, T. VandenDriessche, Universal allogeneic CAR T cells engineered with Sleeping Beauty transposons and CRISPR-CAS9 for cancer immunotherapy. Mol Ther 30, 3155-3175 (2022).
41. Y. Kagoya et al., Genetic Ablation of HLA Class I, Class II, and the T-cell Receptor Enables Allogeneic T Cells to Be Used for Adoptive T-cell Therapy. Cancer Immunol Res 8, 926-936 (2020).
42. D. Martinez Bedoya, V. Dutoit, D. Migliorini, Allogeneic CAR T Cells: An Alternative to Overcome Challenges of CAR T Cell Therapy in Glioblastoma. Front Immunol 12, 640082 (2021).
43. S. Jo et al., Endowing universal CAR T-cell with immune-evasive properties using TALEN-gene editing. Nat Commun 13, 3453 (2022).
44. S. Depil, P. Duchateau, S. A. Grupp, G. Mufti, L. Poirot, ‘Off-the-shelf’ allogeneic CAR T cells: development and challenges. Nat Rev Drug Discov 19, 185-199 (2020).
45. B. Silva-Santos, K. Serre, H. Norell, gammadelta T cells in cancer. Nat Rev Immunol 15, 683-691 (2015).
46. J. C. Ribot, N. Lopes, B. Silva-Santos, gammadelta T cells in tissue physiology and surveillance. Nat Rev Immunol 21, 221-232 (2021).
47. J. H. Park, H. K. Lee, Function of gammadelta T cells in tumor immunology and their application to cancer therapy. Exp Mol Med 53, 318-327 (2021).
48. N. L. de Vries et al., gammadelta T cells are effectors of immunotherapy in cancers with HLA class I defects. Nature 613, 743-750 (2023).
49. T. Saito et al., Surface expression of only gamma delta and/or alpha beta T cell receptor heterodimers by cells with four (alpha, beta, gamma, delta) functional receptor chains. J Exp Med 168, 1003-1020 (1988).
50. F. Koning, W. L. Maloy, D. Cohen, J. E. Coligan, Independent association of T cell receptor beta and gamma chains with CD3 in the same cell. J Exp Med 166, 595-600 (1987).
51. A. Makkouk et al., Off-the-shelf Vdelta1 gamma delta T cells engineered with glypican-3 (GPC-3)-specific chimeric antigen receptor (CAR) and soluble IL-15 display robust antitumor efficacy against hepatocellular carcinoma. J Immunother Cancer 9, (2021).
52. M. Legut, D. K. Cole, A. K. Sewell, The promise of gammadelta T cells and the gammadelta T cell receptor for cancer immunotherapy. Cell Mol Immunol 12, 656-668 (2015).
53. L. Peng et al., Monospecific and bispecific monoclonal SARS-CoV-2 neutralizing antibodies that maintain potency against B.1.617. Nat Commun 13, 1638 (2022).
54. J. Chng et al., Cleavage efficient 2A peptides for high level monoclonal antibody expression in CHO cells. MAbs 7, 403-412 (2015).
55. X. Dai et al., One-step generation of modular CAR-T cells with AAV-Cpf1. Nat Methods 16, 247-254 (2019).
56. D. Killock, GPRC5D-CAR T cells active in MM. Nat Rev Clin Oncol 19, 747 (2022).
57. X. Wang et al., A transgene-encoded cell surface polypeptide for selection, in vivo tracking, and ablation of engineered cells. Blood 118, 1255-1263 (2011).
58. T. L. Roth et al., Pooled Knockin Targeting for Genome Engineering of Cellular Immunotherapies. Cell 181, 728-744 e721 (2020).
59. Y. Kagoya et al., A novel chimeric antigen receptor containing a JAK-STAT signaling domain mediates superior antitumor effects. Nat Med 24, 352-359 (2018).
60. S. C. Oostindie, G. A. Lazar, J. Schuurman, P. Parren, Avidity in antibody effector functions and biotherapeutic drug design. Nat Rev Drug Discov 21, 715-735 (2022).
61. T. T. Hansel, H. Kropshofer, T. Singer, J. A. Mitchell, A. J. George, The safety and side effects of monoclonal antibodies. Nat Rev Drug Discov 9, 325-338 (2010).
62. V. H. Trang et al., A coiled-coil masking domain for selective activation of therapeutic antibodies. Nat Biotechnol 37, 761-765 (2019).
63. A. Mansurov et al., Masking the immunotoxicity of interleukin-12 by fusing it with a domain of its receptor via a tumour-protease-cleavable linker. Nat Biomed Eng 6, 819-829 (2022).
64. X. Han et al., Masked Chimeric Antigen Receptor for Tumor-Specific Activation. Mol Ther 25, 274-284 (2017).
65. R. A. Gardner et al., Intent-to-treat leukemia remission by CD19 CAR T cells of defined formulation and dose in children and young adults. Blood 129, 3322-3331 (2017).
66. T. J. Fry et al., CD22-targeted CAR T cells induce remission in B-ALL that is naive or resistant to CD19-targeted CAR immunotherapy. Nat Med 24, 20-28 (2018).
67. J. H. Park et al., Long-Term Follow-up of CD19 CAR Therapy in Acute Lymphoblastic Leukemia. N Engl J Med 378, 449-459 (2018).
68. C. Rafaniello et al., Tisagenlecleucel in Children and Young Adults: Reverse Translational Research by Using Real-World Safety Data. Pharmaceuticals (Basel) 13, (2020).
69. R. Gardner et al., Acquisition of a CD19-negative myeloid phenotype allows immune escape of MLL-rearranged B-ALL from CD19 CAR-T-cell therapy. Blood 127, 2406-2410 (2016).
70. E. Jacoby et al., CD19 CAR immune pressure induces B-precursor acute lymphoblastic leukaemia lineage switch exposing inherent leukaemic plasticity. Nat Commun 7, 12320 (2016).
71. H. Wang, X. Song, L. Shen, X. Wang, C. Xu, Exploiting T cell signaling to optimize engineered T cell therapies. Trends Cancer 8, 123-134 (2022).
72. P. F. Robbins et al., A pilot trial using lymphocytes genetically engineered with an NY-ESO-1-reactive T-cell receptor: long-term follow-up and correlates with response. Clin Cancer Res 21, 1019-1027 (2015).
73. M. Leisegang et al., Eradication of Large Solid Tumors by Gene Therapy with a T-Cell Receptor Targeting a Single Cancer-Specific Point Mutation. Clin Cancer Res 22, 2734-2743 (2016).
74. K. Kudo et al., T lymphocytes expressing a CD16 signaling receptor exert antibody-dependent cancer cell killing. Cancer Res 74, 93-103 (2014).
75. Y. C. Kuo et al., Antibody-based redirection of universal Fabrack-CAR T cells selectively kill antigen bearing tumor cells. J Immunother Cancer 10, (2022).
76. K. Urbanska et al., A universal strategy for adoptive immunotherapy of cancer through use of a novel T-cell antigen receptor. Cancer Res 72, 1844-1852 (2012).
77. K. Tamada et al., Redirecting gene-modified T cells toward various cancer types using tagged antibodies. Clin Cancer Res 18, 6436-6445 (2012).
78. M. S. Kim et al., Redirection of genetically engineered CAR-T cells using bifunctional small molecules. J Am Chem Soc 137, 2832-2835 (2015).
79. Y. Cao ei al., Design of Switchable Chimeric Antigen Receptor T Cells Targeting Breast Cancer. Angew Chem Int Ed Engl 55, 7520-7524 (2016).
80. K. Urbanska et al., Targeted cancer immunotherapy via combination of designer bispecific antibody and novel gene-engineered T cells. J Transl Med 12, 347 (2014).
81. C. H. Karches et al., Bispecific Antibodies Enable Synthetic Agonistic Receptor-Transduced T Cells for Tumor Immunotherapy. Clin Cancer Res 25, 5890-5900 (2019).
82. B. Clemenceau et al., Antibody-dependent cellular cytotoxicity (ADCC) is mediated by genetically modified antigen-specific human T lymphocytes. Blood 107, 4669-4677 (2006).
83. F. Ochi et al., Gene-modified human alpha/beta-T cells expressing a chimeric CD16-CD3zeta receptor as adoptively transferable effector cells for anticancer monoclonal antibody therapy. Cancer Immunol Res 2, 249-262 (2014).
84. H. Tanaka et al., Development of Engineered T Cells Expressing a Chimeric CD16-CD3zeta Receptor to Improve the Clinical Efficacy of Mogamulizumab Therapy Against Adult T-Cell Leukemia. Clin Cancer Res 22, 4405-4416 (2016).
85. F. Rataj et al., High-affinity CD16-polymorphism and Fc-engineered antibodies enable activity of CD16-chimeric antigen receptor-modified T cells for cancer therapy. Br J Cancer 120, 79-87 (2019).
86. J. Mansilla-Soto et al., HLA-independent T cell receptors for targeting tumors with low antigen density. Nat Med 28, 345-352 (2022).
87. G. M. Bendle et al., Lethal graft-versus-host disease in mouse models of T cell receptor gene therapy. Nat Med 16, 565-570, 561p following 570 (2010).
88. M. Legut, G. Dolton, A. A. Mian, O. G. Ottmann, A. K. Sewell, CRISPR-mediated TCR replacement generates superior anticancer transgenic T cells. Blood 131, 311-322 (2018).

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives. Thus, for example, a list of different gene targets does not indicate that the listed gene targets are obvious one to the other, nor is it an admission of equivalence or obviousness.

Every component disclosed herein is intended to be and should be considered to be specifically disclosed herein. Further, every subgroup that can be identified within this disclosure is intended to be and should be considered to be specifically disclosed herein. As a result, it is specifically contemplated that any component, or subgroup of components can be either specifically included for or excluded from use or included in or excluded from a list of components.

Those skilled in the art will recognize, or be able to ascertain using no more than routine experimentation, many equivalents to the specific forms of the method and compositions described herein. Such equivalents are intended to be encompassed by the following claims.

1. A chimeric gamma delta T-cell receptor (γδTCR) comprising constant and variable immunoglobulin domains,
wherein the constant immunoglobulin domains (IgC) comprise a gamma TCR constant domain and a delta TCR constant domain,
wherein the variable immunoglobulin domains comprise an immunoglobulin antigen binding variable heavy domain (IgVh), and an immunoglobulin antigen binding variable light domain (IgVl),
wherein the antigen binding domains specifically bind constant domain(s) of human immunoglobulin IgG.

2. The chimeric γδTCR of claim 1, comprising one or more one or more IgC comprising the amino acid sequence of any one or more of SEQ ID NOs:1-2, and light and heavy chain variable regions including complementarity determining regions (CDRs) of SEQ ID NOS:3 and/or 4, or a functional variant having at least 75% sequence identity to any one or more of SEQ ID NOs:1-4
optionally wherein the CDRs comprise one or more of SEQ ID NOS:27-32.

3. The chimeric γδTCR of claim 2, comprising all of SEQ ID NOs:1-4.

4. The chimeric γδTCR of claim 1, further comprising a removable masking moiety that prevents the antigen binding domains specifically binding to constant domain(s) of human immunoglobulin IgG.

5. The chimeric γδTCR of claim 4, wherein the masking moiety comprises a coiled-coil peptide selected from the group consisting of CC2B, CC3, CC4, and CC5.

6. The chimeric γδTCR of claim 4, wherein the masking moiety comprises a protease-cleavable masking moiety that is removed in the presence of a protease enzyme.

7. The chimeric gamma delta TCR receptor of claim 6, wherein the protease comprises a urokinase.

8. The chimeric γδTCR of claim 4, wherein the masking moiety comprises LSGRSDNH (SEQ ID NO:5).

9. The chimeric γδTCR of claim 1, further comprising one or more intracellular domain(s) of a costimulatory molecule selected from CD27, CD28, CD137, 0X40, IL2Rβ, ICOS, IL7Rα, CD30, CD40, CD3, LFA 1, ICOS, CD2, CD7, LIGHT, NKG2C, B7 H3, and ligands of CD83.

10. The chimeric γδTCR of claim 9, comprising the intracellular domain of CD28 associated with the delta TCR constant domain.

11. The chimeric γδTCR of claim 9, comprising the intracellular domain of any one of 0X40, IL2Rβ, ICOS, or IL7Rα, associated with the gamma TCR constant domain.

12. (canceled)

13. A nucleic acid encoding or expressing the chimeric γδTCR of claim 1.

14. (canceled)

15. A cell comprising the chimeric γδTCR of claim 1.

16. A genetically modified T-cell expressing the chimeric γδTCR of claim 1 at the cell surface,
wherein the genetically modified T-cell is activated upon binding of the variable antigen binding domains to the constant domain(s) of human immunoglobulin IgG.

17. The cell of any one of claim 16 comprising one or more additional genetic modifications in a gene selected from the group consisting of TRAC, TRBC, B2M and CIITA.

18. (canceled)

19. (canceled)

20. A pharmaceutical composition comprising a population of cells derived by expanding the cell of claim 16, a pharmaceutically acceptable buffer, carrier, diluent or excipient, and one or more clones of human monoclonal antibodies

21. (canceled)

22. (canceled)

23. A method of treating a subject having a disease, disorder, or condition comprising administering to the subject an effective amount of the pharmaceutical composition of claim 20.

24.-31. (canceled)

32. A kit comprising the nucleic acid of claim 13 optionally wherein the kit further comprises a human monoclonal antibody.

33. An antigen binding protein
(i) that specifically binds to GPRC5D, comprising:
(a) a light chain variable region comprising CDRs of SEQ ID NO:19,
optionally wherein the CDRs comprise the amino acid sequence set forth in one or more of SEQ ID NOS:24-26,
or
a light chain variable region of SEQ ID NO:19 or a variant thereof with at least 70% sequence identity thereto; and
(b) a heavy chain variable region comprising CDRs of SEQ ID NO:17; optionally wherein the CDRs comprise the amino acid sequence set forth in one or more of SEQ ID NOS:21-23,
or
a heavy chain variable region of SEQ ID NO:17 or a variant thereof with at least 70% sequence identity thereto;
or a combination thereof, or an antigen-binding fragment or variant thereof; or
(ii) that specifically binds to human IgG1, comprising:
(a) a light chain variable region comprising CDRs of SEQ ID NO:3,
optionally wherein the CDRs comprise the amino acid sequence set forth in one or more of SEQ ID NOS:27-29, or
a light chain variable region of SEQ ID NO:3, or a variant thereof with at least 70% sequence identity thereto;
(b) a heavy chain variable region comprising CDRs of SEQ ID NO:4, optionally wherein the CDRs comprise the amino acid sequence set forth in one or more of SEQ ID NOS:30-32: or
a heavy chain variable region of SEQ ID NO:4 or a variant thereof with at least 70% sequence identity thereto;
or a combination thereof, or an antigen-binding fragment or variant thereof.

34.-40. (canceled)

41. A chimeric gamma delta T-cell receptor (γδTCR) comprising
a polypeptide comprising the amino acid sequence of SEQ ID NO:6 or a variant thereof with at least 70% sequence identity thereto, optionally comprising one or more complementarity determining region (CDR) comprising the amino acids set forth in one or more of SEQ ID NOS:27-29, and
a polypeptide comprising the amino acid sequence of SEQ ID NO:7 or a variant thereof with at least 70% sequence identity thereto,
optionally comprising one or more complementarity determining region (CDR) comprising the amino acids set forth in one or more of SEQ ID NOS:30-32.
